SEQUENCE LISTING (9 sequences)

SEQ ID NO: 1 - protein, 8 aa, Artificial sequence
  Synthetic
LEHHHHHH

SEQ ID NO: 2 - protein, 313 aa, Artificial sequence
  Synthetic
SGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQSGVTFQLEHHHHHH

SEQ ID NO: 3 - protein, 308 aa, Artificial sequence
  Synthetic
HMSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQ

SEQ ID NO: 4 - DNA, 39 bp, Artificial sequence
  Synthetic
gcggtctcaaggttcaggatttaggaagatggcatttcc

SEQ ID NO: 5 - DNA, 38 bp, Artificial sequence
  Synthetic
gctctagattactgaaaggtcacgccgctgcattgacg

SEQ ID NO: 6 - protein, 306 aa, Artificial sequence [sequence omitted]

SEQ ID NO: 7 - protein, 324 aa, Artificial sequence
  Synthetic
GEVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKLEHHHHHH

SEQ ID NO: 8 - protein, 14 aa, Artificial sequence
  Synthetic
KTSAVLQSGFRKME

SEQ ID NO: 9 - protein, 11 aa, Artificial sequence
  Synthetic
FTLRGGAPTKV

(19) United States
(12) Patent Application Publication
(10) Pub. No.: US 2024/0083885 A1
(43) Pub. Date: Mar. 14, 2024

(54) COMPOSITIONS AND METHODS FOR INHIBITING M PRO PROTEASE ACTIVITY AND FOR PREVENTING AND TREATING SARS-COV-2 INFECTION

(71) Applicant: Arizona Board of Regents on Behalf of the University of Arizona, Tucson, AZ (US)

(72) Inventors: Jun Wang, Tucson, AZ (US); Naoya Kitamura, Tucson, AZ (US); Chunlong Ma, Tucson, AZ (US)

(21) Appl. No.: 18/265,335

(22) PCT Filed: Nov. 29, 2021
(86) PCT No.: PCT/US2021/060923

§ 371 Date: Jun. 5, 2023

Related U.S. Application Data
(60) Provisional application No. 63/121,753, filed on Dec. 4, 2020.
(60) Provisional application No. 63/253,843, filed on Oct. 8, 2021.

(51) Int. Cl. C07D 405/12; A61K 31/4706; A61K 31/573; A61K 31/706; A61K 45/06; C07D 401/12; C07D 405/14; C07D 413/12

(52) U.S. Cl. CPC C07D 405/12; A61K 31/4706; A61K 31/573; A61K 31/706; A61K 45/06; C07D 401/12; C07D 405/14; C07D 413/12

(57) ABSTRACT

This invention is in the field of medicinal chemistry and relates to a new class of small-molecules having a methyl-acetamido-propanamide structure (e.g., Formula I) which function as inhibitors of the SARS-CoV-2 main protease (M^{pro}), which function as therapeutics for the treatment of viral infection characterized with M^{pro} protease activity and/or expression (e.g., COVID-19), and which function as therapeutics for the treatment of other conditions characterized with M^{pro} protease activity and/or expression.

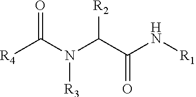

### CROSS-REFERENCE TO RELATED APPLICATIONS

The present application claims priority to U.S. Provisional Patent Application No. 63/121,753, filed Dec. 4, 2020, and U.S. Provisional Patent Application No. 63/253,843, filed Oct. 8, 2021, which are hereby incorporated by reference in their entireties.

### STATEMENT REGARDING FEDERALLY SPONSORED RESEARCH OR DEVELOPMENT

This invention was made with government support under Grant Nos. AI147325 and AI157046, awarded by National Institutes of Health. The government has certain rights in the invention.

### FIELD OF THE INVENTION

This invention is in the field of medicinal chemistry and relates to a new class of small-molecules having a methyl-acetamido-propanamide structure

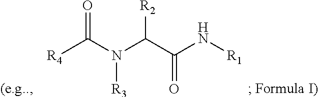

which function as inhibitors of the SARS-CoV-2 main protease (M^{pro}), which function as therapeutics for the treatment of viral infection characterized with M^{pro} protease activity and/or expression (e.g., COVID-19), and which function as therapeutics for the treatment of other conditions characterized with M^{pro} protease activity and/or expression.

### INTRODUCTION

The COVID-19 pandemic has a significant impact on global economy and public health, and there is an urgent need of therapeutic interventions. Encouraging progress has been made in developing mRNA vaccines including the Pfizer BNT162b2 and Moderna mRNA-1273. For small molecule antivirals, the viral polymerase inhibitor remdesivir gained FDA approval on Oct. 22, 2020. Among the other drug targets that are being explored at different stages of preclinical and clinical development, the viral main protease (M^{pro}), also called 3-chymotrypsin-like protease (3CL^{pro}), is one of the high profile antiviral drug targets. M^{pro} is a cysteine protease encoded in the viral polyprotein as non-structural protein 5 (Nsp5) that cleaves the viral polyproteins pp1a and pp1ab at more than 11 sites. Despite its multiple proteolytic sites, M^{pro} was previously shown to have a high substrate specificity of glutamine at the P1 position in the substrate¹. As such, majority of the reported M^{pro} inhibitors were designed to contain a 2-pyrrolidone at the P1 position as a mimetic of the glutamine in the substrate. Most advanced M^{pro} inhibitors including PF-07304814², GC376^{3,4} and 6j⁵ all belong to this category. PF-07304814, an α-hydroxyl ketone prodrug, is developed by Pfizer, which has optimal pharmacokinetic properties in humans and recently entered human clinical trials². GC376 has in vivo antiviral efficacy in treating cats infected with lethal feline infectious peritonitis virus^{6,7}. Recently, the GC376 analog 6j was shown to protect mice from MERS-CoV infection⁵. These results highlights the translational potential of M^{pro} inhibitors.

Improved pharmaceutical agents capable of inhibiting Mpro protease activity are desparately needed. Improved therapies for treating COVID-19 and conditions characterized with M^{pro} protease activity are desperately needed.

The present invention addresses these needs.

### SUMMARY

Through a screening of a focused library of protease inhibitors, it was recently discovered several non-canonical SARS-CoV-2 M^{pro} inhibitors including boceprevir, and calpain inhibitors II, XII³. These inhibitors differ from classic M^{pro} inhibitors such as GC376 in that their P1 substitution does not contain a glutamine mimetic. The co-crystal structures of calpain inhibitors II and XII with SARS-CoV-2 M^{pro} revealed a critical hydrogen bond between the methionine side chain from calpain inhibitor II and pyridyl from calpain inhibitor XII with the H163 side chain imidazole at the S1 pocket⁴. Similarly, the carbonyl from the pyrrolidone in GC376 also forms a hydrogen bond with the H163 side chain imidazole³.

Given the importance of this hydrogen bond with H163 for the high affinity binding of inhibitors to SARS-CoV-2 M^{pro}, it was hypothesized that non-covalent inhibitors without a reactive warhead targeting the C145, but retain the hydrogen bond capacity with H163 can be designed as potent SARS-CoV-2 M^{pro} inhibitors. Indeed, experiments conducted during the course of developing embodiments for the present invention identified the structure-based design of non-covalent M^{pro} inhibitors based on the overlaying structures of SARS-CoV or SARS-CoV-2 M^{pro} in complex with existing inhibitors or the peptide substrate. The design was based on the scaffold of ML188®, a non-covalent SARS-CoV M^{pro} inhibitor, which similarly contains a pyridyl in the P1 substitution. The overlaying structures revealed a strategy of extending the P2 and P4 substitutions in ML188® to fill in the extra space in the S2 and S4 pockets of SARS-CoV-2 M^{pro} as a means to increase the binding affinity. The most potent inhibitor from this study 23R (Jun8-76-3A) showed enzymatic inhibition and cellular antiviral activity similar to the covalent inhibitor GC376. Its mechanism of action was studied in the thermal shift-binding assay and native mass spectrometry binding assay. X-ray crystal structure of SARS-CoV-2 M^{pro} in complex with 23R (Jun8-76-3A) was solved, providing a molecular level understanding of the high binding affinity.

Additional experiments were conducted pertaining to the rational design of covalent SARS-CoV-2 M^{pro} inhibitors with novel cysteine reactive warheads including dichloroacetamide, dibromoacetamide, tribromoacetamide, 2-bromo-2, 2-dichloroacetamide, and 2-chloro-2, 2-dibromoacetamide. Promising lead candidates Jun9-62-2R (dichloroacetamide) and Jun9-88-6R (tribromoacetamide) had not only potent enzymatic inhibition and antiviral activity, but also significantly improved target specificity. Compared to GC-376, these new compounds did not inhibit the host cysteine proteases including calpain I, cathepsin B, cathepsin K, cathepsin L, and caspase-3. Such compounds represent one of the most selective covalent M^{pro} inhibitors reported thus far. The co-crystal structures of SARS-CoV-2 M^{pro} with Jun9-62-2R and Jun9-57-3R reaffirmed the design hypothesis, indicating that both compounds form a covalent bond with the catalytic C145. Overall, such novel compounds represent valuable chemical probes for target validation and drug candidates for further development as SARS-CoV-2 antivirals.

Accordingly, the present invention relates to small-molecules having a methyl-acetamido-propanamide structure which function as inhibitors of the SARS-CoV-2 main protease (M^{pro}), which function as therapeutics for the treatment of viral infection characterized with M^{pro} protease activity and/or expression (e.g., COVID-19), and which function as therapeutics for the treatment of other conditions characterized with M^{pro} protease activity and/or expression.

In a particular embodiment, compounds encompassed within the following formula are provided:

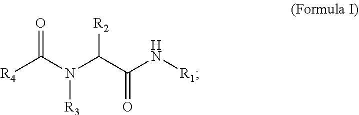

including pharmaceutically acceptable salts, solvates, and/or prodrugs thereof.

Formula I is not limited to a particular chemical moiety for R1, R2, R3, and R4. In some embodiments, the particular chemical moiety for R1, R2, R3, and R4 independently include any chemical moiety that permits the resulting compound to inhibit M^{pro} protease activity. In some embodiments, the particular chemical moiety for R1, R2, R3, and R4 independently include any chemical moiety that permits the resulting compound to prevent viral infection (e.g., COVID-19 infection).

Such embodiments are not limited to a particular definition for R1.

In some embodiments, R1 is selected from hydrogen, methyl,

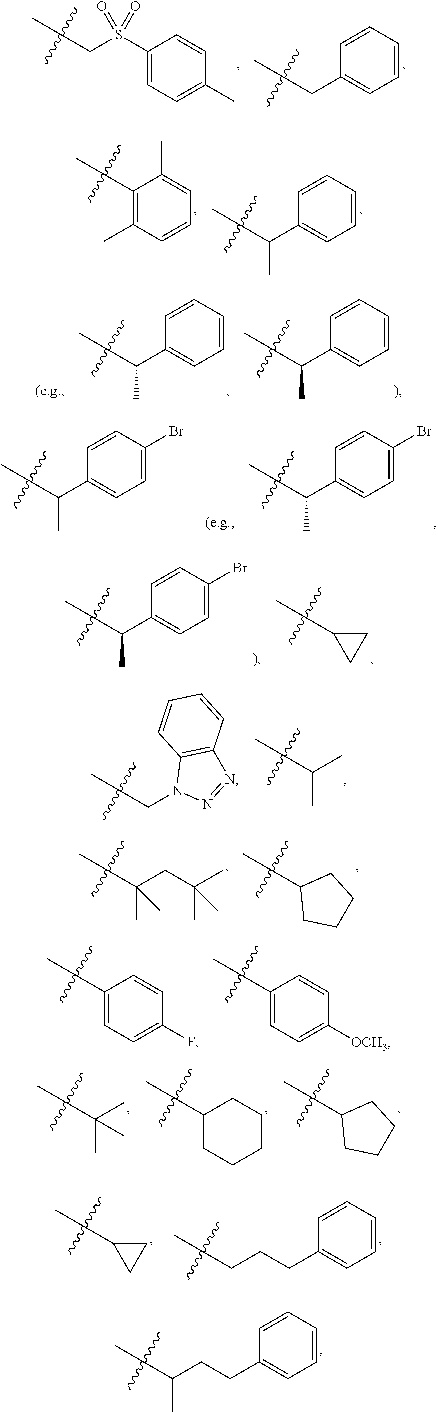

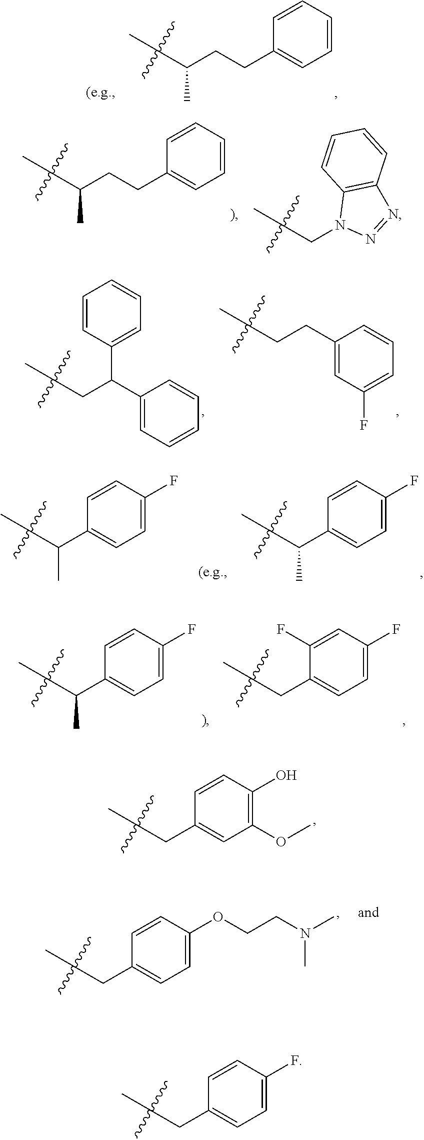

Such embodiments are not limited to a particular definition for R2.

In some embodiments, R2 is selected from hydrogen,

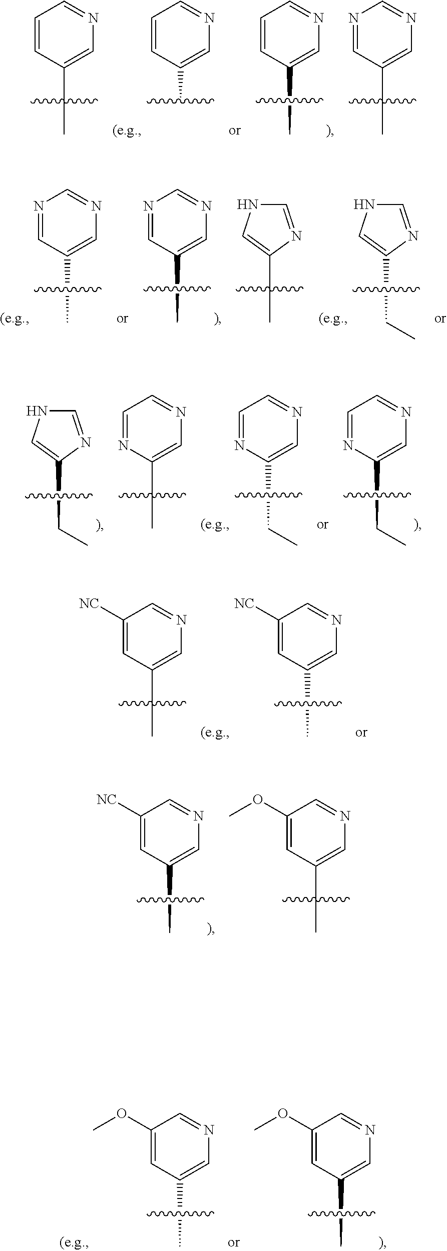

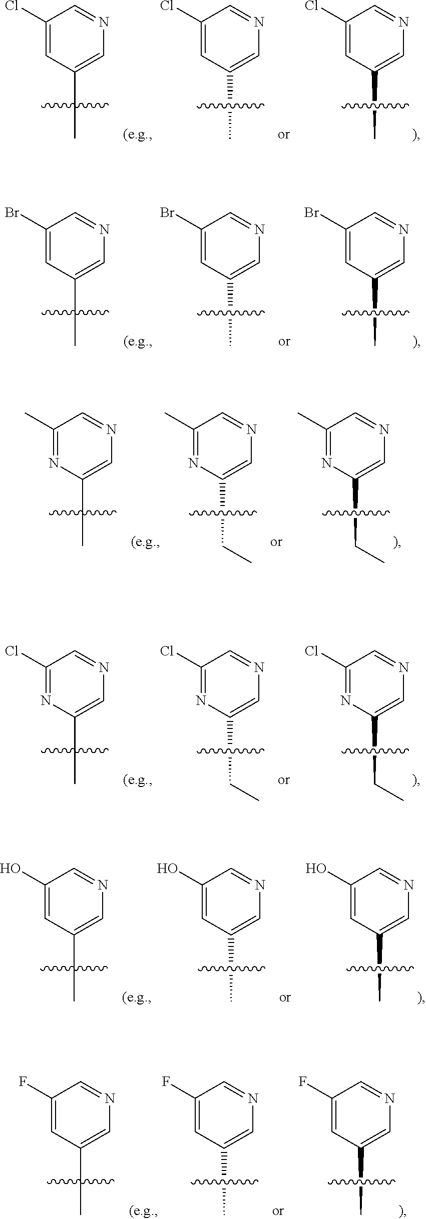

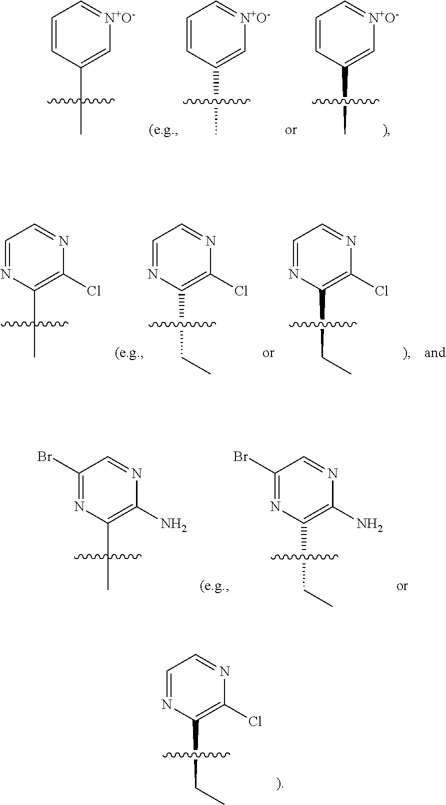

Such embodiments are not limited to a particular definition for R3.

In some embodiments, R3 is selected from hydrogen,

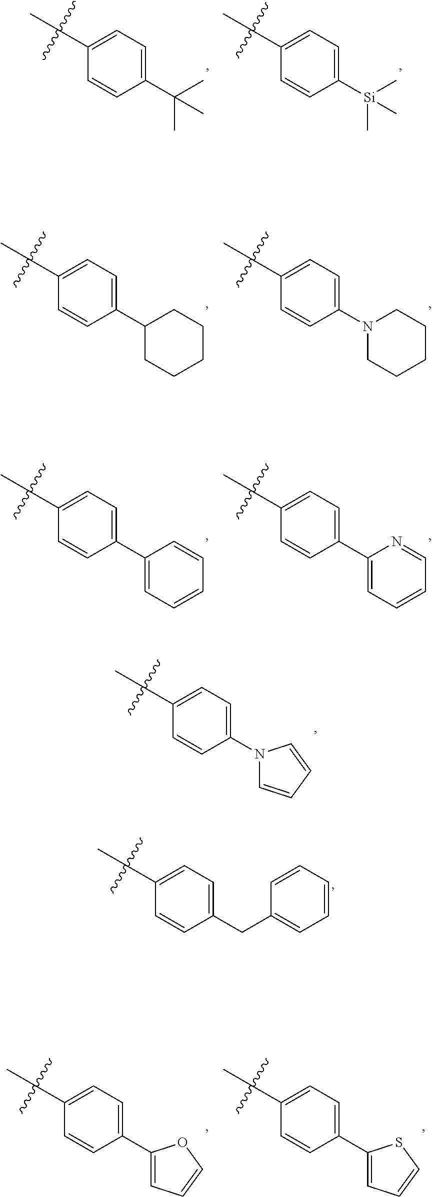

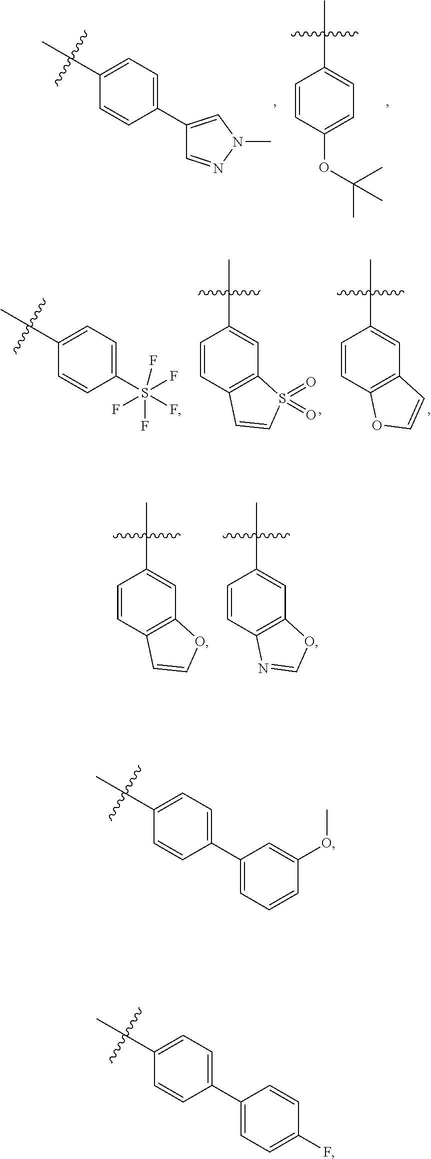

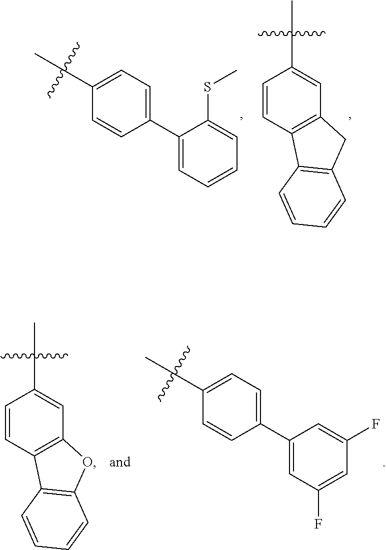

Such embodiments are not limited to a particular definition for R4.

In some embodiments, R4 is selected from hydrogen,

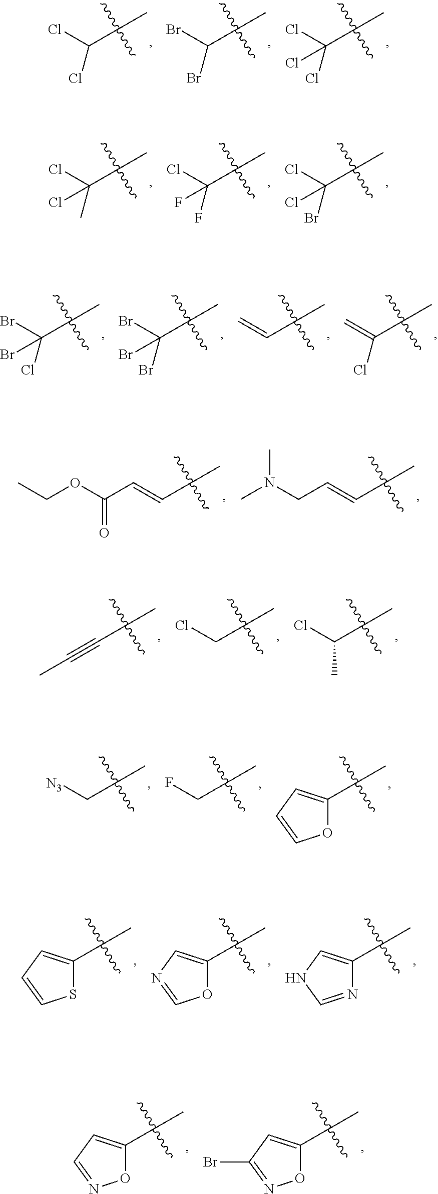

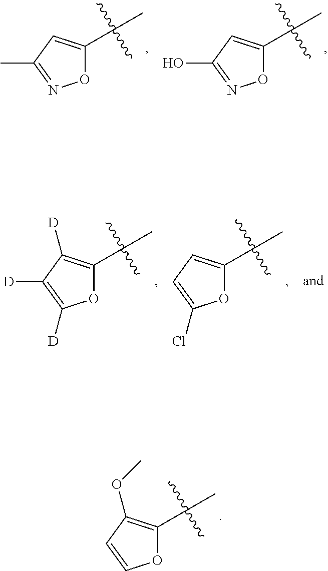

In some embodiments, the compound is recited in Table 1 (these compounds were designed based on a literature reported compound Jun8-48-1 (J. Med. Chem. 2013, 56, 534-546), FIG. 4 (see, Examples I-V), FIG. 10 (see, Example VI and VII), FIG. 11 (see, Example VI and VII), and/or FIG. 18 (see, Example VI and VII).

Through structure-based drug design, the compounds shown in Table 1 were shown to have a more than 20-fold improvement in activity against M^{pro} protease activity. All the following compounds were first tested in the in vitro protease enzymatic assay and active compounds were further tested in the cellular antiviral activity.

TABLE 1

| | SARS-COV-2 | |
|---|---|---|
| | main | SARS-COV-2 antiviral |
| | protease | activity using |
| | enzymatic | immunofluorescence |
| Compounds | inhibition | assay |
| 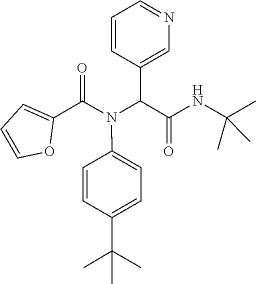 [SMILES: CC(C)(C)NC(=O)C(c1cccnc1)N(C(=O)c1ccco1)c1ccc(C(C)(C)C)cc1] Jun8-48-1 (see, J. Med. Chem. 2013, 56, 534-546) | IC₅₀ = 11.0 μM | |
| 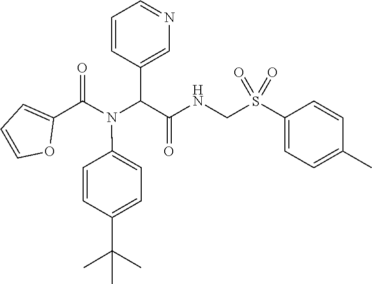 [SMILES: Cc1ccc(S(=O)(=O)CNC(=O)C(c2cccnc2)N(C(=O)c2ccco2)c2ccc(C(C)(C)C)cc2)cc1] Jun8-50-3 | IC₅₀ > 20 μM | |
| 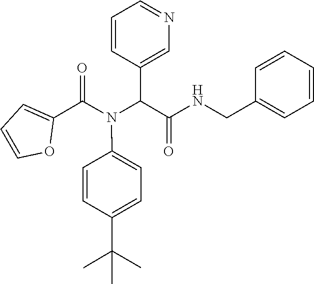 [SMILES: CC(C)(C)c1ccc(N(C(=O)c2ccco2)C(C(=O)NCc2ccccc2)c2cccnc2)cc1] Jun8-50-2 | IC₅₀ = 13.2 μM | |
| 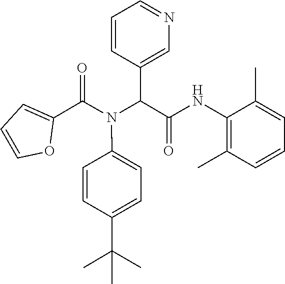 [SMILES: Cc1cccc(C)c1NC(=O)C(c1cccnc1)N(C(=O)c1ccco1)c1ccc(C(C)(C)C)cc1] Jun8-50-4 | IC₅₀ > 20 μM | |
| 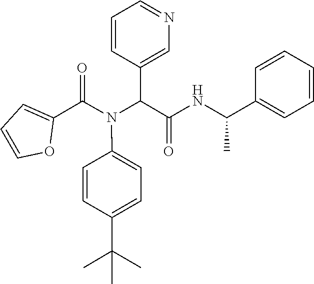 [SMILES: C[C@H](NC(=O)C(c1cccnc1)N(C(=O)c1ccco1)c1ccc(C(C)(C)C)cc1)c1ccccc1] Jun8-56-1 | IC₅₀ = 5.70 μM | EC₅₀ = 17.2 μM |
| 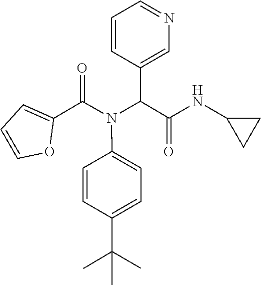 [SMILES: CC(C)(C)c1ccc(N(C(=O)c2ccco2)C(C(=O)NC2CC2)c2cccnc2)cc1] Jun8-58-2 | IC₅₀ = 3.86 μM | |
| 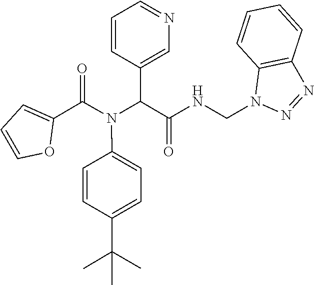 [SMILES: CC(C)(C)c1ccc(N(C(=O)c2ccco2)C(C(=O)NCn2nnc3ccccc32)c2cccnc2)cc1] Jun8-74-1 | IC₅₀ = 8.13 μM | |
| 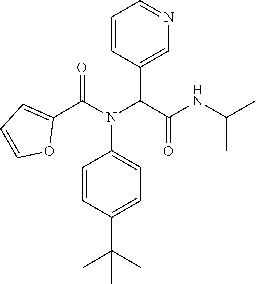 [SMILES: CC(C)NC(=O)C(c1cccnc1)N(C(=O)c1ccco1)c1ccc(C(C)(C)C)cc1] Jun8-88-1 | IC₅₀ = 5.37 μM | |
| 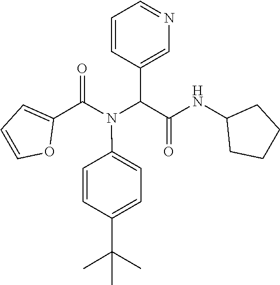 [SMILES: CC(C)(C)c1ccc(N(C(=O)c2ccco2)C(C(=O)NC2CCCC2)c2cccnc2)cc1] Jun8-88-2 | IC₅₀ = 2.51 μM | EC₅₀ = 12.93 μM |
| 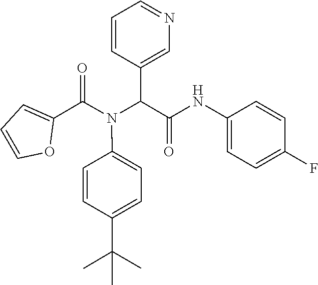 [SMILES: CC(C)(C)c1ccc(N(C(=O)c2ccco2)C(C(=O)Nc2ccc(F)cc2)c2cccnc2)cc1] Jun9-88-3 | IC₅₀ = 7.81 μM | |
| 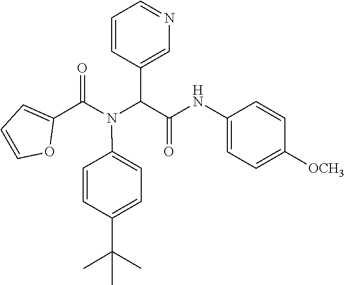 [SMILES: COc1ccc(NC(=O)C(c2cccnc2)N(C(=O)c2ccco2)c2ccc(C(C)(C)C)cc2)cc1] Jun9-88-4 | IC₅₀ = 7.49 μM | |
| 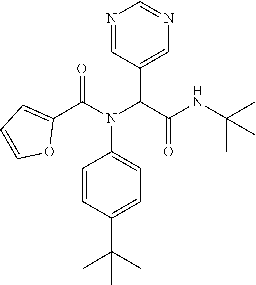 [SMILES: CC(C)(C)NC(=O)C(c1cncnc1)N(C(=O)c1ccco1)c1ccc(C(C)(C)C)cc1] Jun8-58-3 | IC₅₀ > 20 μM | |
| 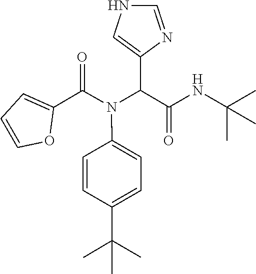 [SMILES: CC(C)(C)NC(=O)C(c1c[nH]cn1)N(C(=O)c1ccco1)c1ccc(C(C)(C)C)cc1] Jun8-59-2 | IC₅₀ > 20 μM | |
| 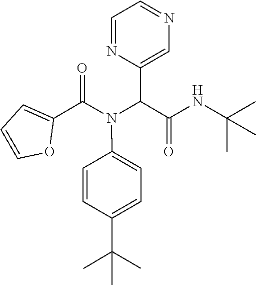 [SMILES: CC(C)(C)NC(=O)C(c1cnccn1)N(C(=O)c1ccco1)c1ccc(C(C)(C)C)cc1] Jun9-17-2 | IC₅₀ = 4.93 μM | |
| 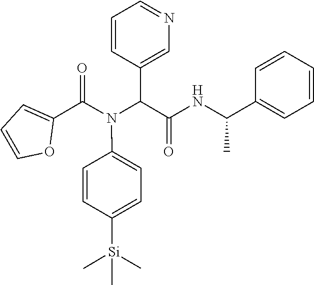 [SMILES: C[C@H](NC(=O)C(c1cccnc1)N(C(=O)c1ccco1)c1ccc([Si](C)(C)C)cc1)c1ccccc1] Jun8-75-2 | IC₅₀ = 1.97 μM | EC₅₀ = 8.84 μM |
| 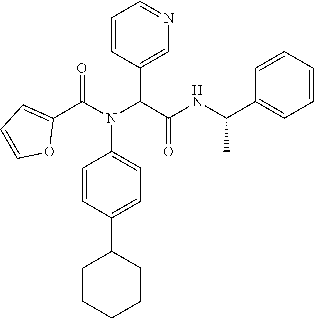 [SMILES: C[C@H](NC(=O)C(c1cccnc1)N(C(=O)c1ccco1)c1ccc(C2CCCCC2)cc1)c1ccccc1] Jun8-66-1 | IC₅₀ = 0.51 μM | |
| 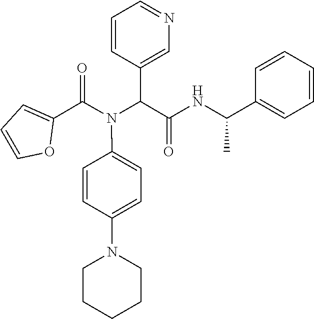 [SMILES: C[C@H](NC(=O)C(c1cccnc1)N(C(=O)c1ccco1)c1ccc(N2CCCCC2)cc1)c1ccccc1] Jun8-97-1 | IC₅₀ = 5.46 μM | |
| 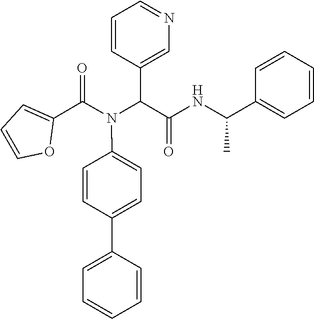 [SMILES: C[C@H](NC(=O)C(c1cccnc1)N(C(=O)c1ccco1)c1ccc(-c2ccccc2)cc1)c1ccccc1] Jun8-76-3 | IC₅₀ = 0.31 μM Jun8-76-3A EC₅₀ = 0.15 μM Jun8-76-3B EC50 = 1.35 μM | EC₅₀ = 1.83 μM Jun8-76-3A EC₅₀ = 1.27 ± 0.09 μM Jun8-76-3B EC₅₀ = 2.97 ± 1.05 μM |
| 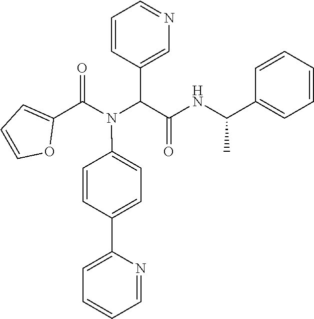 [SMILES: C[C@H](NC(=O)C(c1cccnc1)N(C(=O)c1ccco1)c1ccc(-c2ccccn2)cc1)c1ccccc1] Jun9-6-4 | IC₅₀ = 1.81 μM | |
| 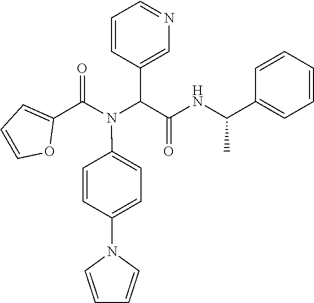 [SMILES: C[C@H](NC(=O)C(c1cccnc1)N(C(=O)c1ccco1)c1ccc(-n2cccc2)cc1)c1ccccc1] Jun8-76-5 | IC₅₀ = 0.94 μM | EC₅₀ = 1.90 μM |
| 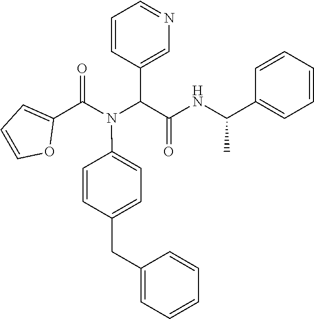 [SMILES: C[C@H](NC(=O)C(c1cccnc1)N(C(=O)c1ccco1)c1ccc(Cc2ccccc2)cc1)c1ccccc1] Jun8-92-4 | IC₅₀ = 35.1 μM | |
| 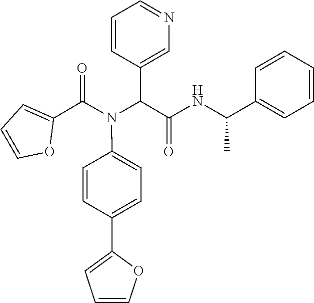 [SMILES: C[C@H](NC(=O)C(c1cccnc1)N(C(=O)c1ccco1)c1ccc(-c2ccco2)cc1)c1ccccc1] Jun8-91-5 | IC₅₀ = 2.54 μM | |
| 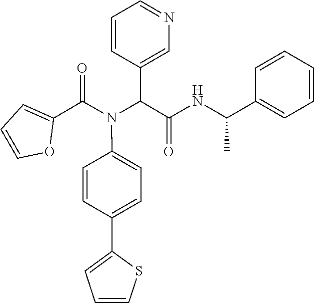 [SMILES: C[C@H](NC(=O)C(c1cccnc1)N(C(=O)c1ccco1)c1ccc(-c2cccs2)cc1)c1ccccc1] Jun8-91-4 | IC₅₀ = 14.6 μM | |
| 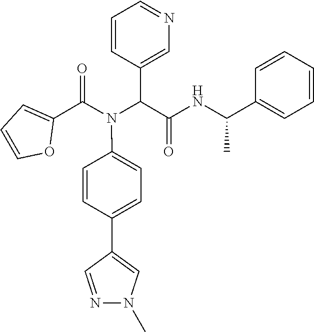 [SMILES: C[C@H](NC(=O)C(c1cccnc1)N(C(=O)c1ccco1)c1ccc(-c2cnn(C)c2)cc1)c1ccccc1] Jun8-91-6 | IC₅₀ = 37.3 μM | |
| 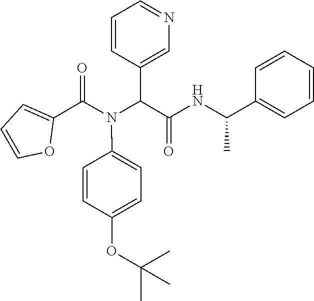 [SMILES: C[C@H](NC(=O)C(c1cccnc1)N(C(=O)c1ccco1)c1ccc(OC(C)(C)C)cc1)c1ccccc1] Jun9-5-4 | IC₅₀ = 21.88 μM | |
| 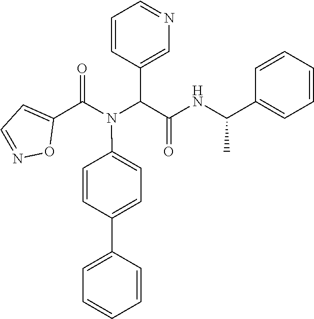 [SMILES: C[C@H](NC(=O)C(c1cccnc1)N(C(=O)c1ccno1)c1ccc(-c2ccccc2)cc1)c1ccccc1] Jun8-92-1 | IC₅₀ = 2.47 μM | |
| 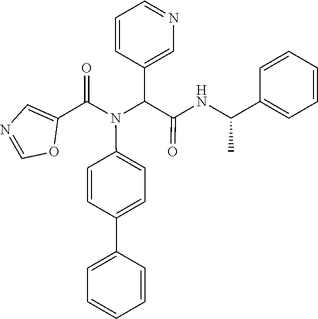 [SMILES: C[C@H](NC(=O)C(c1cccnc1)N(C(=O)c1cnco1)c1ccc(-c2ccccc2)cc1)c1ccccc1] Jun8-92-2 | IC₅₀ = 4.99 μM | |
| 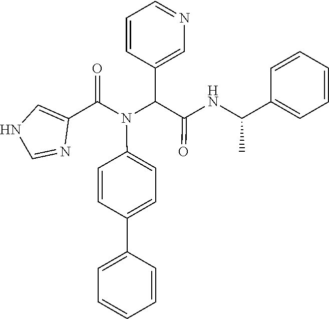 [SMILES: C[C@H](NC(=O)C(c1cccnc1)N(C(=O)c1c[nH]cn1)c1ccc(-c2ccccc2)cc1)c1ccccc1] Jun8-90-3 | IC₅₀ = 0.96 μM | |
| 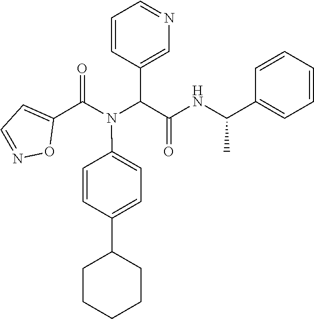 [SMILES: C[C@H](NC(=O)C(c1cccnc1)N(C(=O)c1ccno1)c1ccc(C2CCCCC2)cc1)c1ccccc1] Jun8-76-4 | IC₅₀ = 0.81 μM | |
| 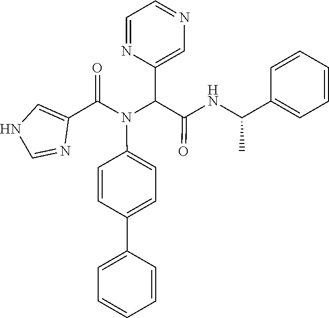 [SMILES: C[C@H](NC(=O)C(c1cnccn1)N(C(=O)c1c[nH]cn1)c1ccc(-c2ccccc2)cc1)c1ccccc1] Jun9-5-2 | IC₅₀ = 0.67 μM | EC₅₀ = 9.89 ± 4.51 μM |
| 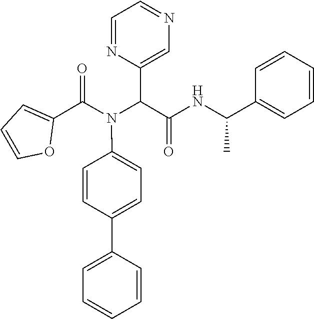 [SMILES: C[C@H](NC(=O)C(c1cnccn1)N(C(=O)c1ccco1)c1ccc(-c2ccccc2)cc1)c1ccccc1] Jun9-4-4 | IC₅₀ = 0.28 μM | EC₅₀ = 1.43 ± 0.41 μM |
| 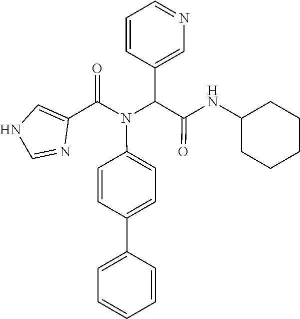 [SMILES: O=C(NC1CCCCC1)C(c1cccnc1)N(C(=O)c1c[nH]cn1)c1ccc(-c2ccccc2)cc1] Jun9-4-2 | IC₅₀ = 5.82 μM | |
| 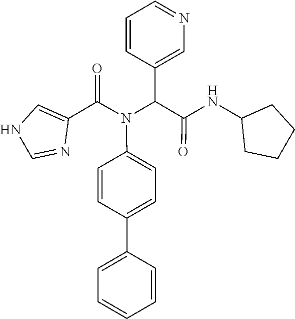 [SMILES: O=C(NC1CCCC1)C(c1cccnc1)N(C(=O)c1c[nH]cn1)c1ccc(-c2ccccc2)cc1] Jun9-3-5 | IC₅₀ = 7.27 μM | |
| 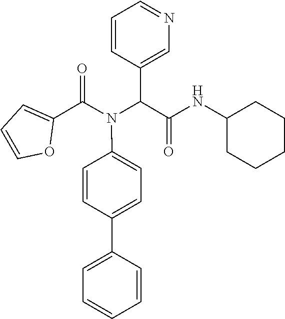 [SMILES: O=C(NC1CCCCC1)C(c1cccnc1)N(C(=O)c1ccco1)c1ccc(-c2ccccc2)cc1] Jun9-3-3 | IC₅₀ = 2.68 μM | |
| 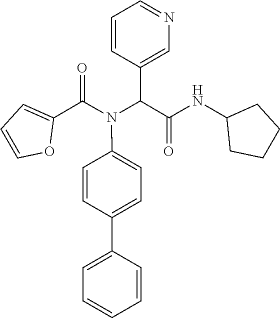 [SMILES: O=C(NC1CCCC1)C(c1cccnc1)N(C(=O)c1ccco1)c1ccc(-c2ccccc2)cc1] Jun9-3-2 | IC₅₀ = 4.10 μM | |
| 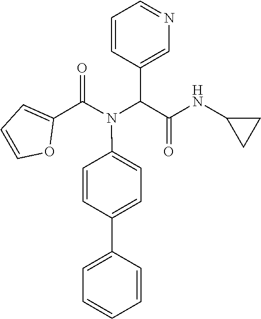 [SMILES: O=C(NC1CC1)C(c1cccnc1)N(C(=O)c1ccco1)c1ccc(-c2ccccc2)cc1] Jun9-3-2 | IC₅₀ = 12.09 μM | |
| 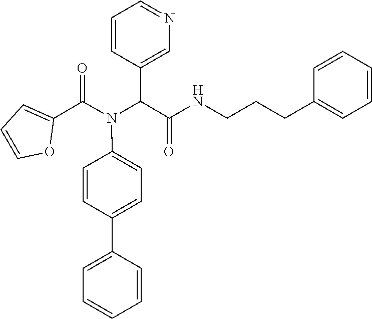 [SMILES: O=C(NCCCc1ccccc1)C(c1cccnc1)N(C(=O)c1ccco1)c1ccc(-c2ccccc2)cc1] Jun9-12-2 | IC₅₀ = 1.06 μM | EC₅₀ = 2.13 ± 0.86 μM |
| 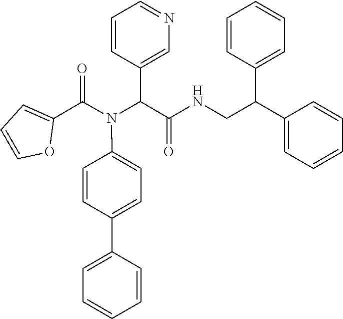 [SMILES: O=C(NCC(c1ccccc1)c1ccccc1)C(c1cccnc1)N(C(=O)c1ccco1)c1ccc(-c2ccccc2)cc1] Jun9-5-3 | IC₅₀ = 1.48 μM | |

The invention further provides processes for preparing any of the compounds of the present invention.

In certain embodiments, the present invention provides methods for administering a pharmaceutical composition comprising one or more compounds of the present invention to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virus having M^{pro} protease activity and/or expression.

In such embodiments, administration of the pharmaceutical composition results in suppression of M^{pro} protease activity within the subject. In some embodiments, administration of the pharmaceutical composition results in suppression of any pathway related activity related to M^{pro} protease activity within the subject.

In some embodiments, the pharmaceutical composition comprising one or more compounds of the present invention is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising one or more compounds of the present invention. In some embodiments, the pharmaceutical composition comprising one or more compounds of the present invention is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition comprising one or more compounds of the present invention. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising one or more compounds of the present invention results in suppression of M^{pro} activity within the cell.

In certain embodiments, the present invention provides kits comprising a pharmaceutical composition comprising one or more compounds of the present invention, and one or more of (1) a container, pack, or dispenser, (2) one or more additional agents selected from hydroxychloroquine, dexamethasone, and remdesivir, and (3) instructions for administration.

### BRIEF DESCRIPTION OF THE DRAWINGS

FIG. 1. Structure of M^{pro} with its inhibitors. An X-ray crystal structure of SARS-CoV-2 M^{pro} in complex with calpain inhibitor XII (PDB: 6XFN). b X-ray crystal structure of SARS-Co-V M^{pro} in complex with ML188® (PDB: 3V3M).

FIG. 2. Design rationale for the non-covalent SARS-CoV-2 M^{pro} inhibitors. An Overlay X-ray crystal structures of SARS-CoV M^{pro}+MDL188® (PDB: 3V3M) and SARS-CoV M^{pro} H41A mutant+peptide substrate (PDB: 2Q6G). b Overlay X-ray crystal structures of SARS-CoV M^{pro}+MDL188® (PDB: 3V3M) and SARS-CoV-2 M^{pro}+GC376 (PDB: 6WTT). c Overlay X-ray crystal structures of SARS-CoV M^{pro}+MDL188® (PDB: 3V3M) and SARS-CoV-2 M^{pro}+UAWJ247 (PDB: 6XFN). d Chemical structures of ML188®, peptide substrate VLQS, GC376, and UAWJ247. f Stepwise optimization of ML188® towards potent non-covalent SARS-CoV-2 M^{pro} inhibitor.

FIG. 3. Synthesis of a focused library of non-covalent SARS-CoV-2 M^{pro} inhibitors. An X-ray crystal structure of SARS-CoV M^{pro}+MDL188® (PDB: 3V3M). b Binding interactions of ML188® with SARS-CoV M^{pro}. c Synthesis of MDL188 analogs using the Ugi four-component reaction.

FIG. 4. Structures of non-covalent SARS-CoV-2 M^{pro} inhibitors and the enzymatic inhibition against SARS-CoV-2 M^{pro}. An Analogs with P3/P4 modifications. b Analogs with P2 modifications. c Analogs with P1′ modifications. d Analogs with P1 modifications. e Analogs with combined P1′, P1, P2, and P3/P4 modifications. f Percentage enzymatic inhibition of SARS-CoV-2 M^{pro} by the designed compounds at 20 μM compound concentration.

FIG. 5. Separation of the two diasteromers of compound 23. The absolute stereochemistry of compound 23R was determined in the co-crystal structure of this diasteromer with SARS-CoV-2 M^{pro}.

FIG. 6. Characterization of binding of 23a to SARS-CoV-2 M^{pro} using the Native mass-spectrometry and thermal shift assay.

FIG. 7. X-ray crystal structure of SARS-CoV-2 M^{pro} in complex with 23R.

FIG. 8. Promising SARS-CoV-2 M^{pro} inhibitors reported in the literature.

FIG. 9. Synthesis route for the covalent SARS-CoV-2 M^{pro} inhibitors through Ugi-4CR. The R and S chirality refers to the chiral center at the pyridine substitution.

FIG. 10. Rational design of covalent SARS-CoV-2 M^{pro} inhibitors based on 23R. (A) X-ray crystal structure of SARS-CoV-2 M^{pro} with 23R (PDB: 7KX5). The distance between the furyl ring and the catalytic cysteine 145 is 3.4 Å. (B) Representative cysteine reactive warheads for covalent labeling of C145. (C) FDA-approved covalent inhibitors. The reactive warheads are colored in magenta. Pfizer compound 12 is a preclinical candidate. (D) Designed covalent SARS-CoV-2 M^{pro} inhibitors. The results are average±standard deviation of three repeats.

FIG. 11. SARS-CoV-2 M^{pro} inhibitors with novel acrylamide and haloacetamide warheads. The results are average±standard deviation of three repeats.

FIG. 12. Pharmacological characterization of the SARS-CoV-2 M^{pro} inhibitors. (A) Curve fittings of the enzymatic kinetic studies of four compounds Jun9-62-2R, Jun9-90-3R, Jun9-90-4R, and Jun9-88-6R against SARS-CoV-2 M^{pro}. (B) Binding of four compounds Jun9-62-2R, Jun9-90-3R, Jun9-90-4R, and Jun9-88-6R to SARS-CoV-2 M^{pro} in the thermal shift assay. (C) Fast dilution experiment. 10 μM M^{pro} was pre-incubated with 10 μM of testing compounds for 2 h at 30° C.; the pre-formed compound-enzyme complex was diluted 100-fold into reaction buffer before initiate the enzymatic reaction. The recovered enzymatic activity was compared with DMSO control. 23R is a non-covalent M^{pro} inhibitor and it was included as a control. (D) Time dependent inhibition of M^{pro} by Jun9-62-2R. 100 nM SARS CoV-2 M^{pro} was pre-incubated with Jun9-62-2R for various period of time (0 min to 2 h) before the addition of 10 μM FRET substrate to initiate the enzymatic reaction. 23R was included as a control. (E-H) Native mass spectrometry assay of SARS-CoV-2 M^{pro} reveals binding of Jun9-62-2R with mass modifications of 482 Da (E), Jun9-89-2R with mass modifications of 526 Da (F), Jun9-88-6R with mass modifications of 526 Da (G), and Jun9-89-4R with mass modifications of (a) 481 and (b) 561 Da (H). M^{pro} functions as a dimer, and both one drug per dimer (Protein+1 Mod) and two drugs per dimer (Protein+2 Mods) were observed. (I) Flip-GFP assay characterization of the inhibition of the cellular enzymatic activity of SARS-CoV-2 M^{pro} by the four compounds Jun9-62-2R, Jun9-90-3R, Jun9-90-4R, and Jun9-88-6R. (J) Curve fittings of the Flip-GFP M^{pro} assay. The results are average±standard deviation of three repeats.

FIG. 13. Antiviral activity of Jun9-62-2R, Jun9-90-3R, Jun9-90-4R, and Jun9-88-6R against SARS-CoV-2 in different cell lines. (A) Antiviral activity against SARS-CoV-2 in Vero E6 cells. (B) Antiviral activity against SARS-CoV-2 in Caco2-hACE2 cells. (C) Antiviral activity of Jun9-90-3R in Calu-3 cells. The results are average±standard deviation of three repeats.

FIG. 14. Target selectivity of SARS-CoV-2 M^{pro} inhibitors against host proteases. (A) Heat map of target selectivity. (B) IC₅₀ values of Jun9-62-2R and Jun9-88-6R against host proteases in the FRET-based enzymatic assay. ^{a}The result was from Hu, Y.; et al., ACS Infect. Dis. 2021, 7 (3), 586-597.

FIG. 15. X-ray crystal structures of SARS-CoV-2 M^{pro} in complex with Jun9-62-2R (A) and Jun9-57-3R (B). 2Fo-Fc electron density map, shown in gray, is contoured at 1σ. Structural superimposition of the noncovalent analogues Jun8-76-3A (white, PDB ID 7KX5) and ML188 (yellow, PDB ID 7L0D) with Jun9-62-2R (C) and Jun9-57-3R (D) reveal a different mode of interaction with the catalytic core.

FIG. 16 shows enzymatic kinetic studies of Jun9-62-2R (A), Jun9-90-3R (B), Jun9-90-4R (C), and Jun9-88-6R (D) in inhibiting SARS-CoV-2 M^{pro}.

FIG. 17. HMNR and CNMR spectra of compounds described in Examples VI and VII.

FIG. 18 shows IC₅₀ values of additional compounds of the invention against host Mpro.

### DETAILED DESCRIPTION OF THE INVENTION

The main protease (M^{pro}) of SARS-CoV-2 is a validated antiviral drug target. Several M^{pro} inhibitors have been reported showing both potent enzymatic inhibition and cellular antiviral activity, including GC376 and its analogs, boceprevir, calpain inhibitors II and XII, all of which contain reactive warheads that covalently modify the catalytic cysteine 145.

Experiments conducted during the course of developing embodiments for the present invention determined the structure-based design of non-covalent MPO inhibitors. The most potent compound 23R had cellular antiviral activity similar to covalent inhibitors such as GC376. The design of non-covalent M^{pro} inhibitors was guided by overlaying the X-ray crystal structure of SARS-CoV M^{pro}+ML188®, a non-covalent inhibitor derived from Ugi four-component reaction, with the X-ray crystal structures of SARS-CoV-2 M^{pro}+calpain inhibitor XII/GC376/UAWJ257. Binding site analysis indicated a strategy of extending the P2 and P4 substitutions in MDL188 to achieve optimal shape complementary with the SARS-CoV-2 M^{pro}. Following lead optimization led to the discovery of the most potent lead compound 23R (Jun8-76-3A), which inhibits the SARS-CoV-2 M^{pro} and SARS-CoV-2 viral replication with IC₅₀ of 0.13 μM and EC₅₀ of YY μM, respectively. The binding and specificity of 23R (Jun8-76-3A) to SARS-CoV-2 M^{pro} were confirmed in thermal shift assay and native mass spectrometry. The co-crystal structure of SARS-CoV-2 M^{pro} with 23R (Jun8-76-3A) confirmed our design hypothesis, showing that the P2 biphenyl and the P3/P4 α-methylbenzyl substitutions fit snuggly into the S2 and S3/S4 pockets, respectively. Overall, such experiments revealed the most potent non-covalent SARS-CoV-2 M^{pro} inhibitors indentified so far with a confirmed mechanism of action.

Additional experiments were conducted pertaining to the rational design of covalent SARS-CoV-2 M^{pro} inhibitors with novel cysteine reactive warheads including dichloroacetamide, dibromoacetamide, tribromoacetamide, 2-bromo-2, 2-dichloroacetamide, and 2-chloro-2, 2-dibromoacetamide. Promising lead candidates Jun9-62-2R (dichloroacetamide) and Jun9-88-6R (tribromoacetamide) had not only potent enzymatic inhibition and antiviral activity, but also significantly improved target specificity. Compared to GC-376, these new compounds did not inhibit the host cysteine proteases including calpain I, cathepsin B, cathepsin K, cathepsin L, and caspase-3. Such compounds represent one of the most selective covalent M^{pro} inhibitors reported thus far. The co-crystal structures of SARS-CoV-2 M^{pro} with Jun9-62-2R and Jun9-57-3R reaffirmed the design hypothesis, indicating that both compounds form a covalent bond with the catalytic C145. Overall, such novel compounds represent valuable chemical probes for target validation and drug candidates for further development as SARS-CoV-2 antivirals. Accordingly, the present invention relates to small-molecules having a methyl-acetamido-propanamide structure which function as inhibitors of the SARS-CoV-2 main protease (M^{pro}), which function as therapeutics for the treatment of viral infection characterized with M^{pro} protease activity and/or expression (e.g., COVID-19), and which function as therapeutics for the treatment of other conditions characterized with M^{pro} protease activity and/or expression.

In a particular embodiment, compounds encompassed within the following formula are provided:

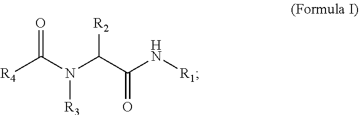

including pharmaceutically acceptable salts, solvates, and/or prodrugs thereof.

Formula I is not limited to a particular chemical moiety for R1, R2, R3, and R4. In some embodiments, the particular chemical moiety for R1, R2, R3, and R4 independently include any chemical moiety that permits the resulting compound to inhibit M^{pro} protease activity. In some embodiments, the particular chemical moiety for R1, R2, R3, and R4 independently include any chemical moiety that permits the resulting compound to prevent viral infection (e.g., COVID-19 infection).

Such embodiments are not limited to a particular definition for R1.

In some embodiments, R1 is selected from hydrogen, methyl,

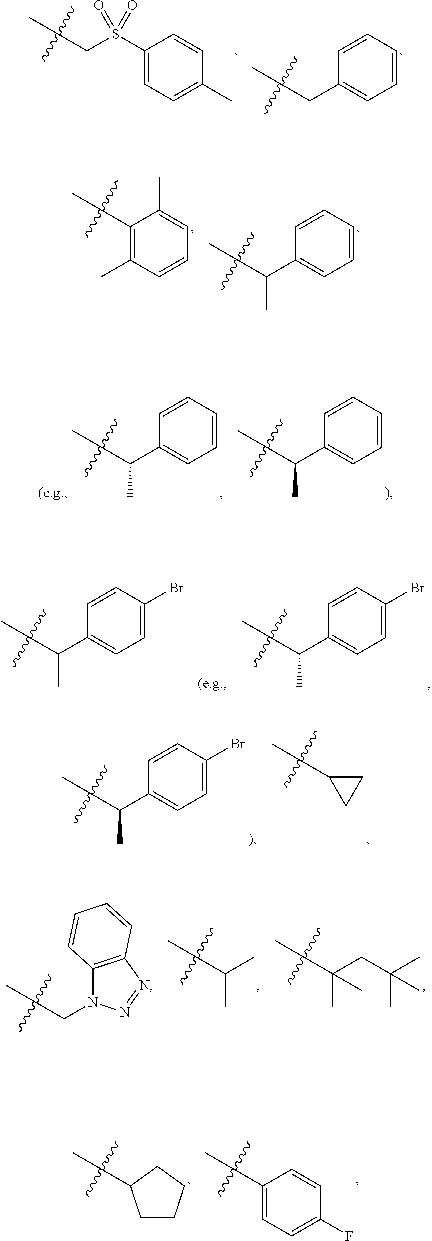

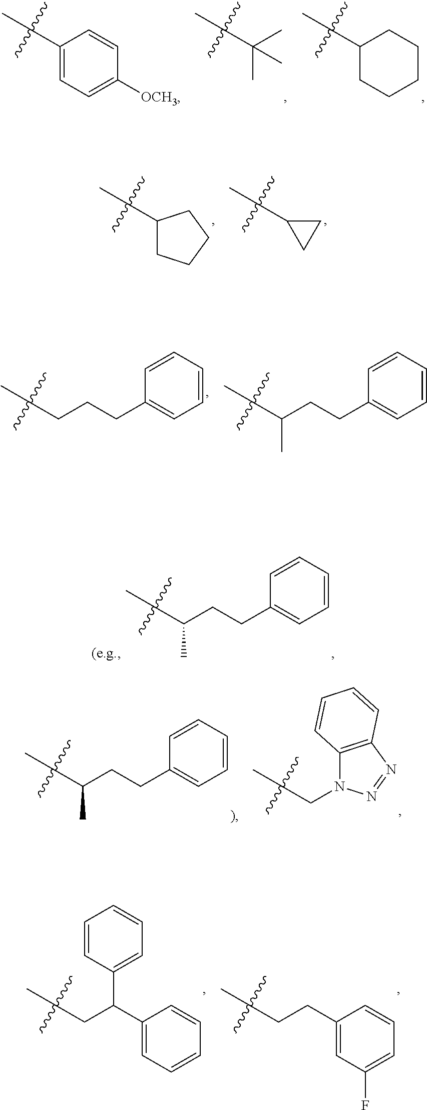

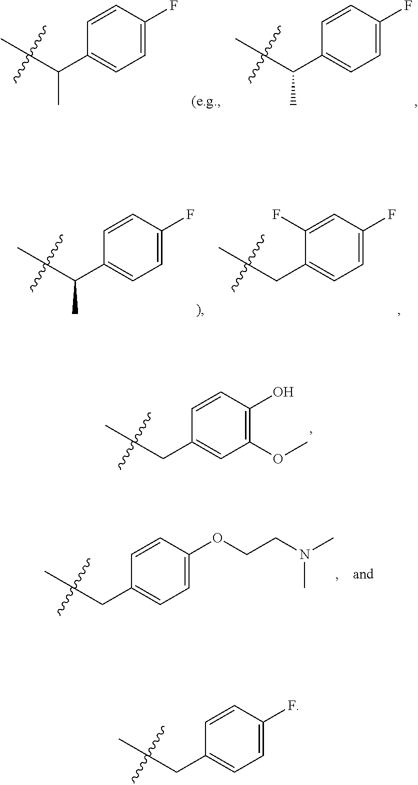

Such embodiments are not limited to a particular definition for R2.

In some embodiments, R2 is selected from hydrogen,

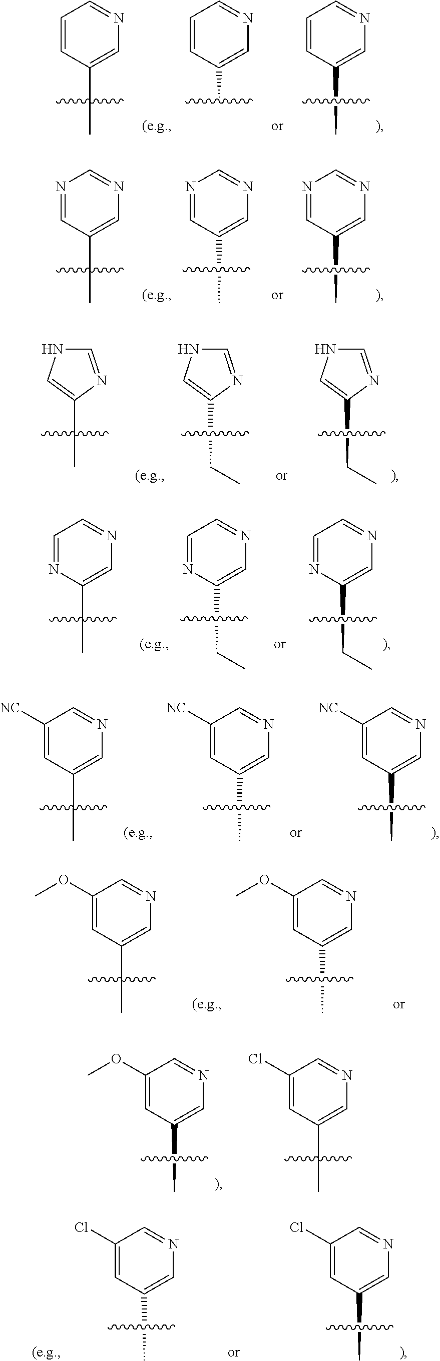

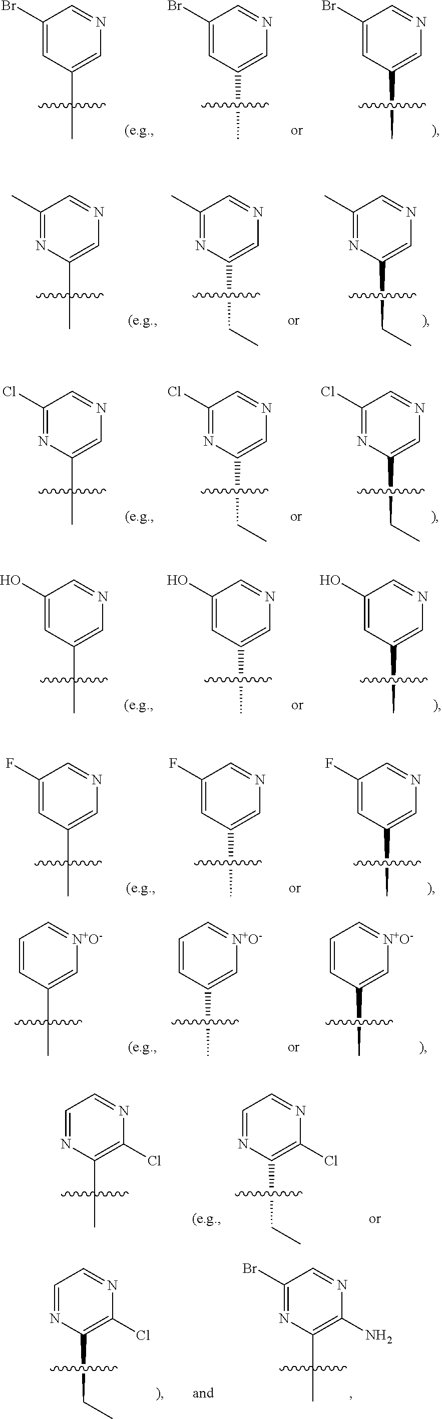

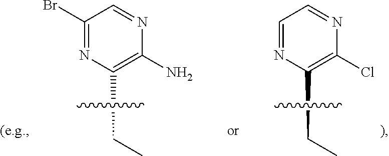

Such embodiments are not limited to a particular definition for R3.

In some embodiments, R3 is selected from hydrogen,

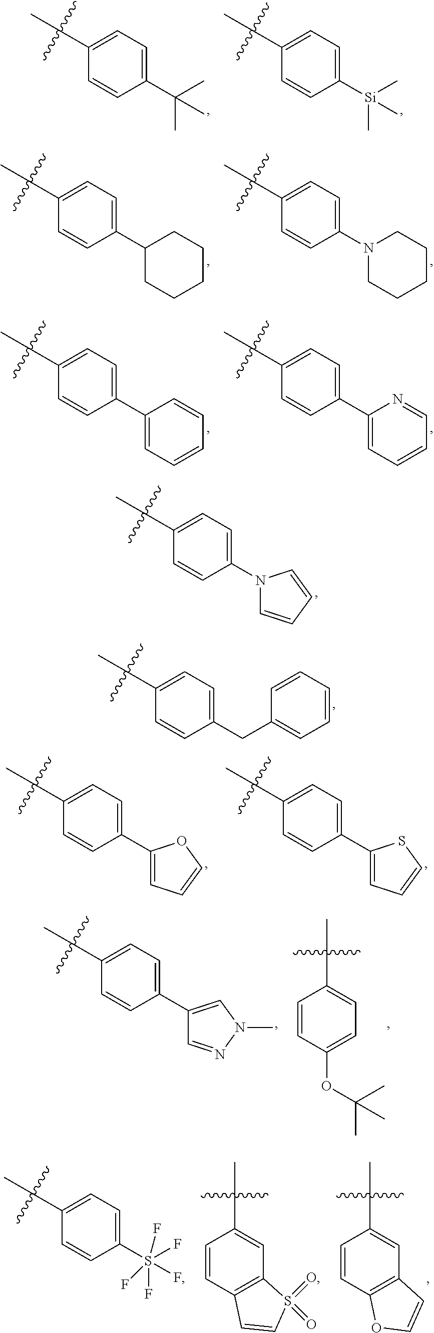

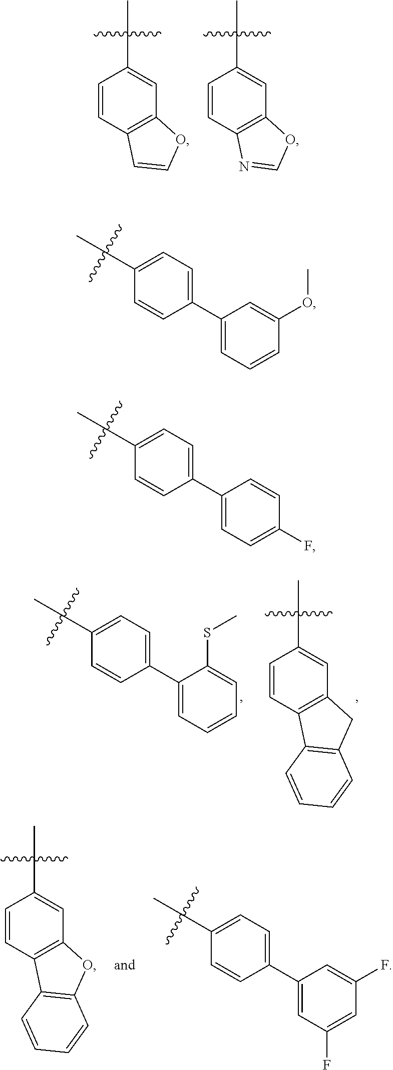

Such embodiments are not limited to a particular definition for R4.

In some embodiments, R4 is selected from hydrogen,

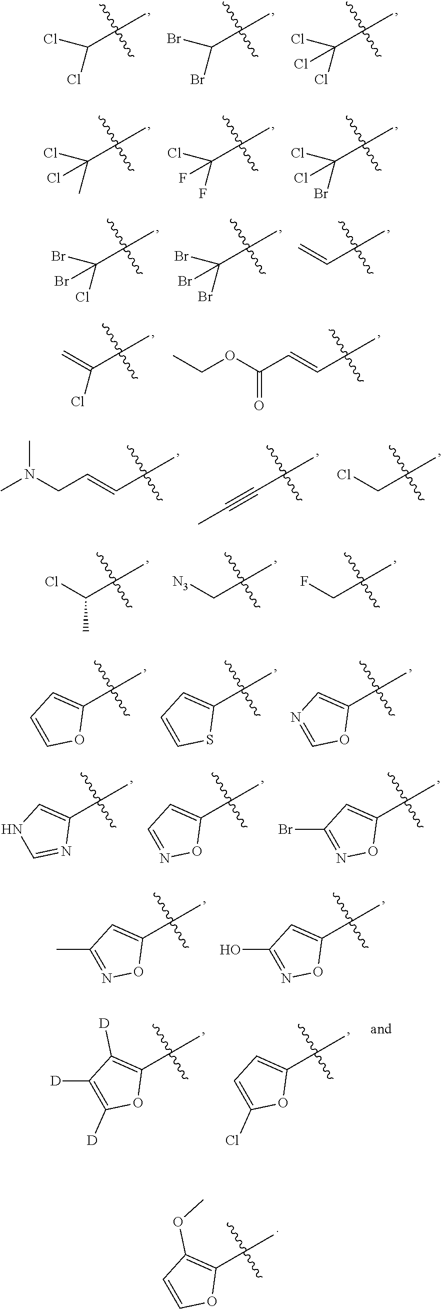

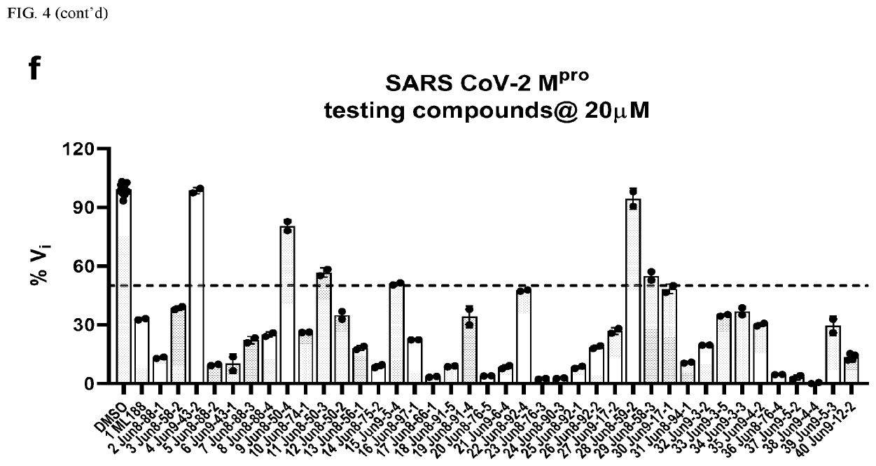

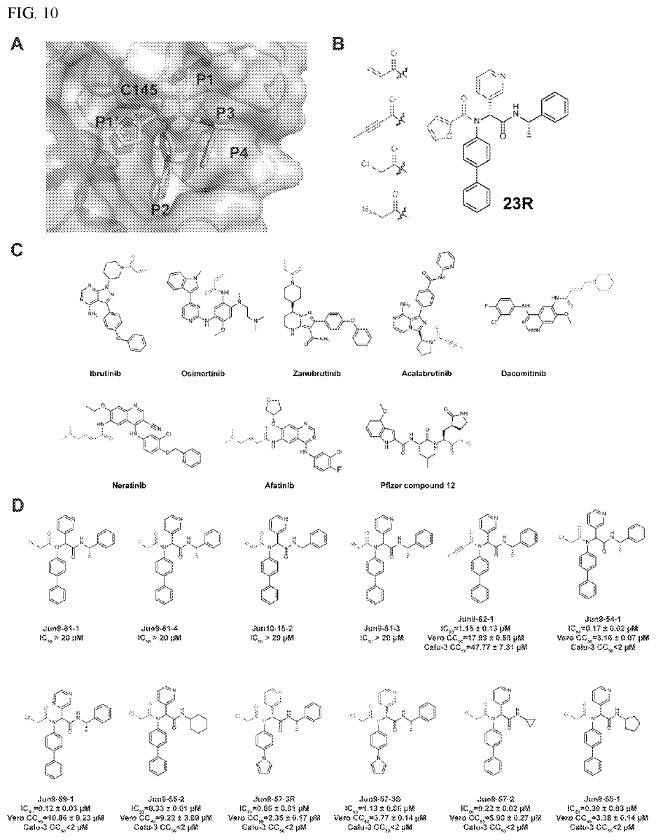

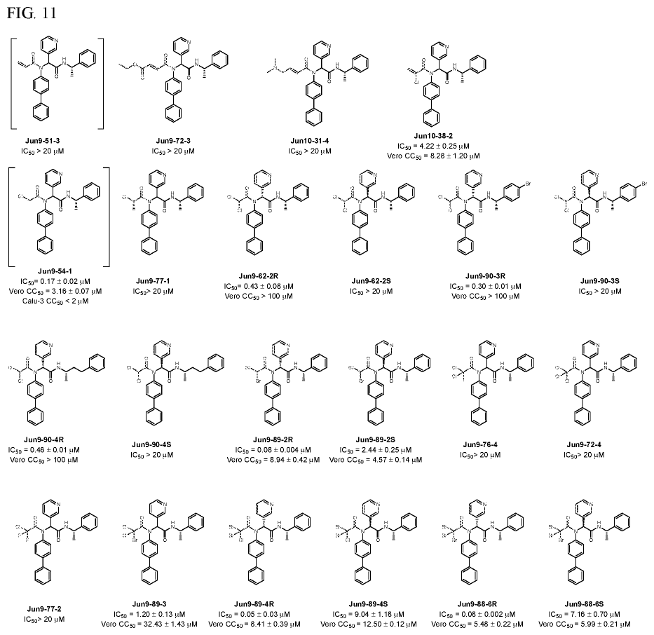

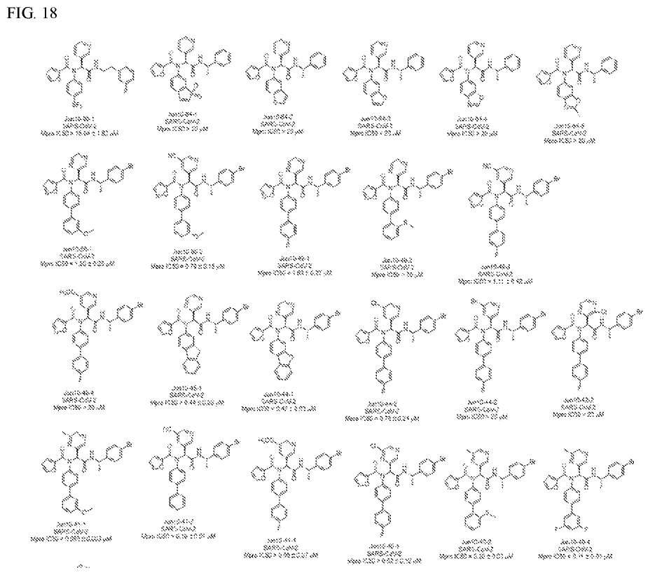

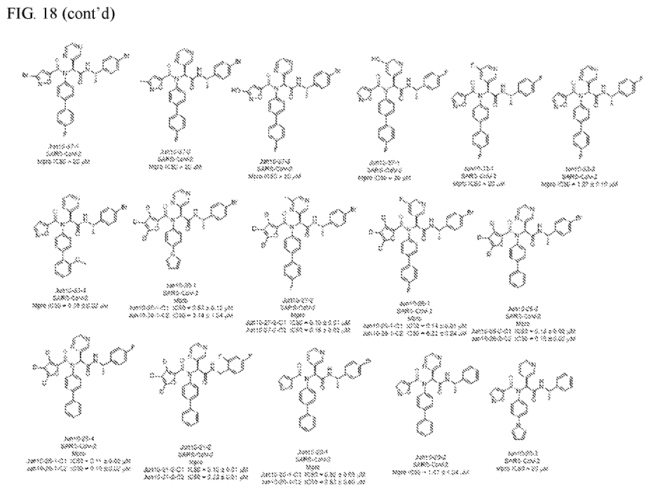

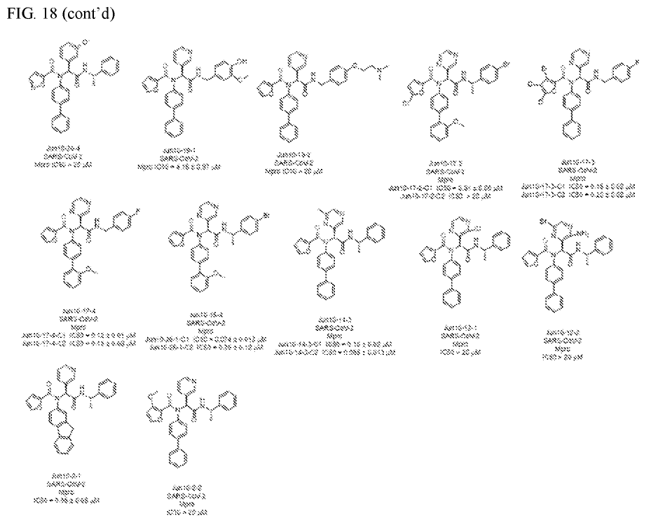

In some embodiments, the compound is recited in Table 1, FIG. 4, FIG. 10, FIG. 11, and/or FIG. 18.

An important aspect of the present invention is that the pharmaceutical compositions comprising one or more of compounds of the present invention are useful in treating viral infection (e.g., SARS-CoV-2 infection) and symptoms related to such a viral infection (e.g., fever, fatigue, dry cough, myalgias, dyspnea, acute respiratory distress syndrome, and pneumonia).

Some embodiments of the present invention provide methods for administering an effective amount of a pharmaceutical composition comprising one or more compounds of the present invention and at least one additional therapeutic agent (including, but not limited to, any pharmaceutical agent useful in treating SARS-CoV-2 infection and/or symptoms related to such a viral infection (e.g., fever, fatigue, dry cough, myalgias, dyspnea, acute respiratory distress syndrome, and pneumonia). In some embodiments, the additional agent is one or more of hydroxychloroquine, dexamethasone, and remdesivir.

In certain embodiments, the present invention provides methods for administering a pharmaceutical composition comprising one or more compounds of the present invention to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virus having M^{pro} protease activity and/or expression.

In such embodiments, administration of the pharmaceutical composition results in suppression of M^{pro} protease activity within the subject. In some embodiments, administration of the pharmaceutical composition results in suppression of any pathway related activity related to M^{pro} protease activity within the subject.

In some embodiments, the pharmaceutical composition comprising one or more compounds of the present invention is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising one or more compounds of the present invention. In some embodiments, the pharmaceutical composition comprising one or more compounds of the present invention is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition comprising one or more compounds of the present invention. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising one or more compounds of the present invention results in suppression of M^{pro} activity within the cell.

In certain embodiments, the present invention provides kits comprising a pharmaceutical composition comprising one or more compounds of the present invention, and one or more of (1) a container, pack, or dispenser, (2) one or more additional agents selected from hydroxychloroquine, dexamethasone, and remdesivir, and (3) instructions for administration.

Compositions within the scope of this invention include all pharmaceutical compositions contained in an amount that is effective to achieve its intended purpose. While individual needs vary, determination of optimal ranges of effective amounts of each component is within the skill of the art. Typically, the pharmaceutical agents which function as inhibitors M^{pro} protease activity may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the inhibiting agent. The unit dose may be administered one or more times daily as one or more tablets or capsules each containing from about 0.1 to about 10 mg, conveniently about 0.25 to 50 mg of the agent (e.g., small molecule) or its solvates.

In a topical formulation, a compound of the present invention (e.g., a comound having a methyl-acetamido-propanamide structure) may be present at a concentration of about 0.01 to 100 mg per gram of carrier. In a one embodiment, such a compound is present at a concentration of about 0.07-1.0 mg/ml, for example, about 0.1-0.5 mg/ml, and in one embodiment, about 0.4 mg/ml.

In addition to administering a compound of the present invention (e.g., a comound having a methyl-acetamido-propanamide structure) as a raw chemical, it may be administered as part of a pharmaceutical preparation containing suitable pharmaceutically acceptable carriers comprising excipients and auxiliaries which facilitate processing of the compound into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of one or more of compounds of the present invention (e.g., comounds having a methyl-acetamido-propanamide structure). Foremost among such patients are mammals, e.g., humans, although the invention is not intended to be so limited. Other patients include veterinary animals (cows, sheep, pigs, horses, dogs, cats and the like).

The pharmaceutical compositions comprising a compound of the present invention (e.g., a comound having a methyl-acetamido-propanamide structure) may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. As used herein, terms such as “I”, “we”, “our”, and similar pronouns refer to the inventors.

### Example I. Rational Design of Non-Covalent SARS-CoV-2 M^{pro} Inhibitors

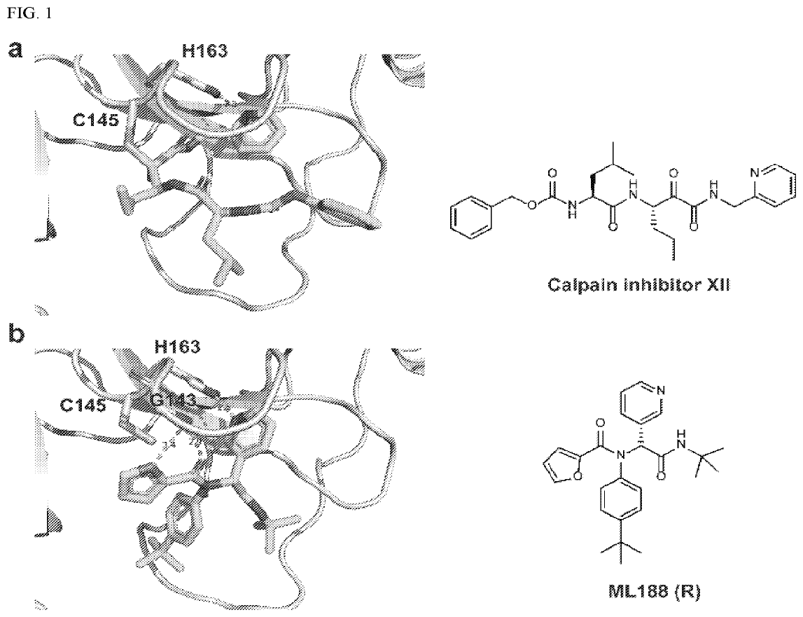

Among the non-canonical SARS-CoV-2 M^{pro} inhibitors we (the inventors of the current application) recently discovered, calpain inhibitor XII has an unexpected binding mode showing an inverted conformation in the active site⁴. Instead of projecting the norvaline and leucine side chains into the S1 and S2 pockets as one would expect from the chemical structure, the pyridyl substitution snuggly fits in the S1 pocket and forms a hydrogen bond with the H163 imidazole (FIG. 1a). This hydrogen bond was proved critical, as replacing the pyridine with benzene led to an analog UAWJ257 with significant loss of enzymatic inhibition⁴. Examining the X-ray crystal structures of SARS-CoV and SARS-CoV-2 M^{pro} in the PDB database revealed another compound ML188®⁸, which shares a similar binding mode with calpain inhibitor XII. ML188® is a non-covalent SARS-CoV M^{pro} inhibitor derived from a hit identified from a high-throughput screening⁸. The pyridine from ML188® similarly fits in the S1 pocket and forms a hydrogen bond with the H163 side chain imidazole (FIG. 1b). In addition, the furyl oxygen and its amide oxygen both form a hydrogen bond with the G143 main chain amide amine. MDL188® was reported to inhibit the SARS-CoV M^{pro} with IC₅₀ value of 1.5±0.3 μM and SARS-CoV viral replication in Vero E6 cells with EC₅₀ value of 14.5 μM⁸.

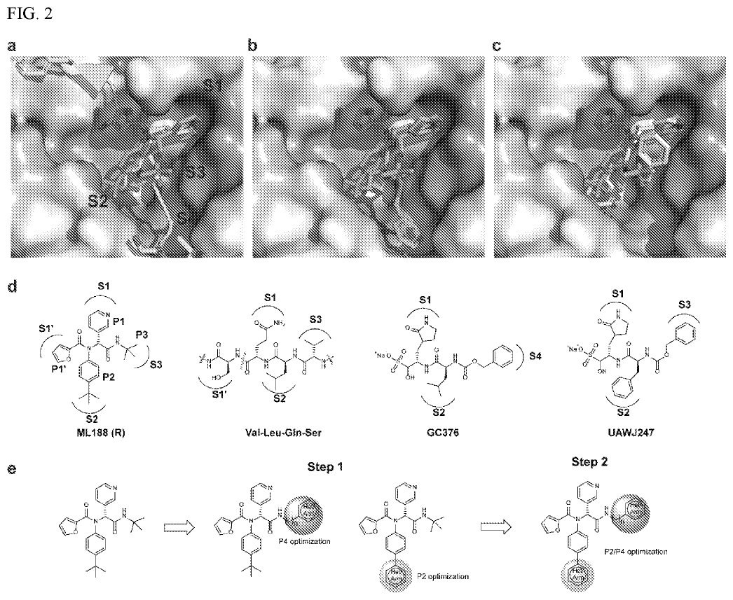

The similar binding mode of ML188® with calpain inhibitor XII, coupled with the convenient synthesis through the one pot Ugi four-component reaction, inspired us to design non-covalent SARS-CoV-2 M^{pro} inhibitors based on the ML188® scaffold. Specifically, we leverage our understanding of the M^{pro} inhibition mechanism based on the X-ray co-crystal structures of SARS-CoV-2 M^{pro} with multiple inhibitors we recently solved to guide the lead optimization (FIGS. 2a-d)^{3,4}. Specifically, overlaying the X-ray crystal structures of SARS-CoV M^{pro}+MDL188® (PDB: 3V3M) and the SARS-CoV M^{pro}H41A mutant+the peptide substrate (PDB: 2Q6G) revealed that the furyl, 4-tert-butylphenyl, pyridyl, and tent-butyl of MDL188® fit in the S1′, S2, S1, and S3 pockets respectively (FIGS. 2a and 2d). Therefore, the furyl, 4-tert-butylphenyl, pyridyl, and tent-butyl substitutions in MDL188® were defined as P1′, P2, P1, and P3, respectively. Next, overlaying the structure of SARS-CoV M^{pro}+MDL188® (PDB: 3V3M) and SARS-CoV-2 M^{pro}+GC376 (PDB: 6WTT) suggested that the tent-butyl at the P3 substitution of ML188® can be extended to fit in the S4 pocket (FIGS. 2b and 2d). Previous structure-activity relationship studies of GC376 indicate that P4 substitution is important, while P3 substitution does not contribute significantly to the binding affinity^{4,5}. Similarly, the overlaying structures of SARS-CoV M^{pro}+MDL188® (PDB: 3V3M) and SARS-CoV-2 M^{pro}+UAWJ247 (PDB: 6XFN) suggested that the 4-tent-butyl at the P2 substitution of ML188® can be replaced by phenyl to occupy the extra space in the S2 pocket (FIGS. 2c and 2d). Overall, the design mainly focuses on extending the P2 and P4 substitutions of ML188® to achieve optimal shape complementarity (FIG. 2e) with the SARS-CoV-2 M^{pro}. In practice, we adopted a stepwise optimization procedure in which the P4 and P2 substitutions were optimized individually in step 1, and then the optimal P2/P4 substitutions were combined in step 2 (FIG. 2e).

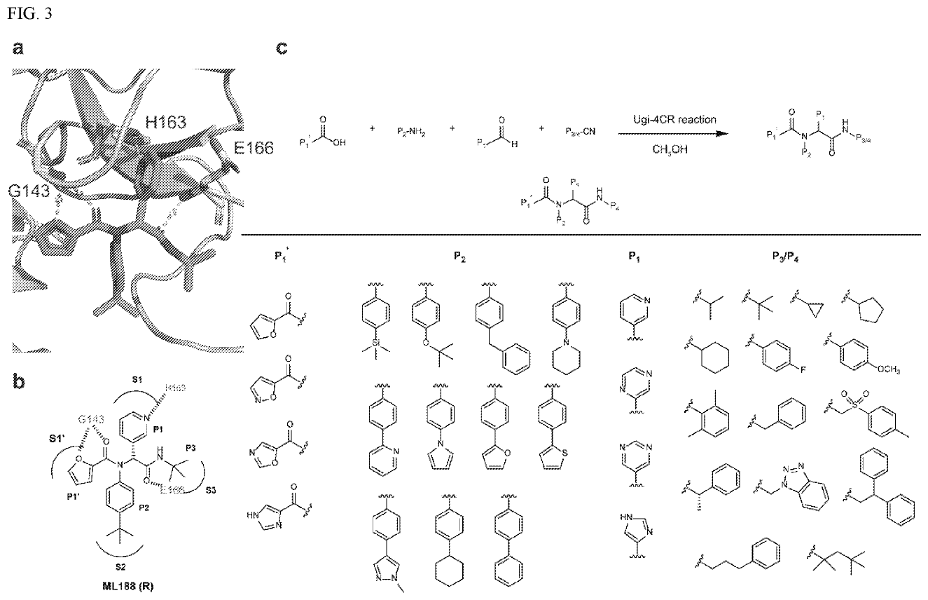

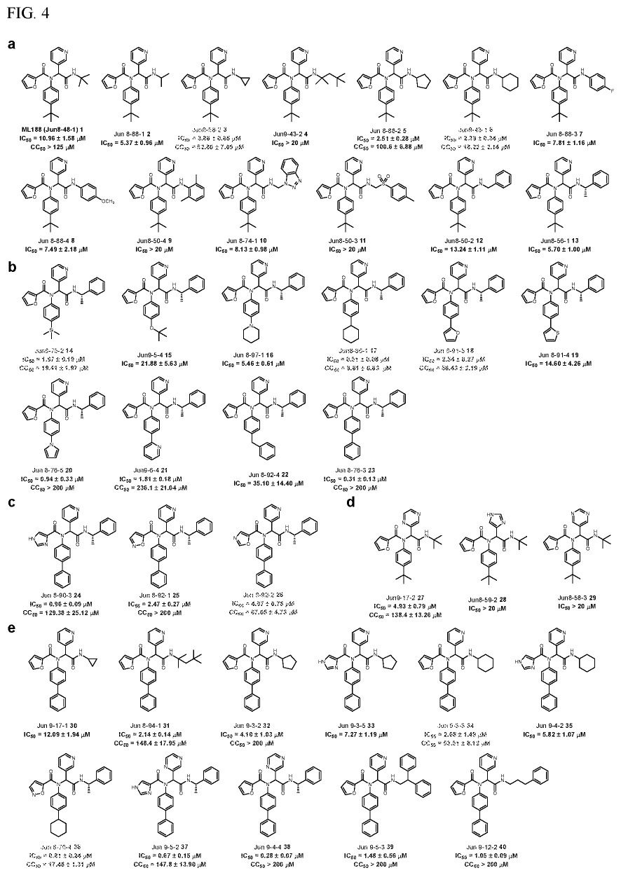

Guided by the design rationale elucidated above, a focused library of MDL188 analogs were designed (FIG. 3). As the P1′ furyl and P1 pyridyl both form a critical hydrogen bond with the M^{pro} (FIGS. 3a-b), the P1′ and P1 substitutions were kept with minimal variation (FIG. 3c). All designed compounds were synthesized using the one pot Ugi four-component reaction and tested as enantiomer/diasteromer mixtures (FIG. 3c). To circumvent the need of relying on expansive chiral HPLC column for the separation of enantiomers, we strategically introduced the chiral isocyanide such that the diasteromer product mixture can be separated by convenient silica gel column or reverse phase HPLC column purification.

### Example II. Structure-Activity Relationship Studies of Non-Covalent SARS-CoV-2 M^{pro} Inhibitors

In total, 39 compounds were synthesized (FIGS. 4a-4e) and all compounds were initially tested in the FRET-based enzymatic assay against SARS-CoV-2 M^{pro} at 20 μM (FIG. 4f). Compounds showing more than 50% inhibition at 20 μM were further titrated to determine the IC₅₀ values. Next, compounds with IC₅₀ values lower than 5 μM were selected for cellular cytotoxicity profiling in Vero E6 cells. The purpose was to prioritize lead candidates for the in vitro cellular antiviral assay with infectious SARS-CoV-2. Compounds with potent enzymatic inhibition (IC₅₀<5 μM) but moderate to high cellular cytotoxicity (CC₅₀<100 μM) were labeled in red. Compounds with both potent enzymatic inhibition (IC₅₀<5 μM) and low cellular cytotoxicity (CC₅₀>100 μM) were labeled in blue. As shown in FIG. 4f, majority of the compounds showed more than 50% inhibition when tested at 20 μM. Specifically, FIG. 4a lists compounds with P4 variations. As a reference, MDL188 (1) (racemic mixture) inhibits SARS-CoV-2 M^{pro} with an IC₅₀ value of 10.96±1.58 μM. It was found that compounds 2, 3, 5, 6, 7, 8, 10, and 13 had improved enzymatic inhibition compared to ML188 (1). These results suggest that : a) isopropyl (2), cyclopropyl (3), cyclopentyl (5), cyclohexyl (6), and phenyl (7 and 8) are the more favorable substitutions at the P3 position than tent-butyl; b) compound 13 with the (S)-α-methylbenzyl substitution at the P3/P4 position had improved potency, which validates the hypothesis that extending the substitutions to the S4 pocket can indeed improve the enzymatic inhibition (FIG. 2b). Given the advantage of convenient separation of diastereomers over enantiomers, we therefore decided to fix the P3/P4 substitution as a-methylbenzyl substitution while optimizing P2 substitutions (FIG. 4b). All compounds in FIG. 4b were designed to have extended substitutions at the 4-position of benzyl to occupy the extra space in the S2 pocket (FIG. 2c). Consistent with the design hypothesis, several compounds including 14, 17, 18, 20, 21, and 23 had significantly improved enzymatic inhibition (IC₅₀<3 μM) compared to compound 13. Replacing the tent-butyl in compound 13 with the bulkier trimethylsilyl led to compound 14 with a 2.9-fold increase in M^{pro} inhibition. Cyclohexyl (17), pyrrolyl (20), pyridyl (21), and phenyl (23) were found to be the most favorable substitutions at the S2 pocket. Compound 16 with piperidyl substitution had similar potency as compound 13, while compounds 15 and 19 with O-tert butyl and 2-thienyl were less active. Further extending the substitution to benzyl led to compound 22 that was inactive, suggesting biphenyl might be the largest substitution that can be accommodated at the S2 pocket.

The P1′ and P1 substitutions (FIGS. 4c and 4d) were chosen to retain the critical hydrogen bonds in MDL188 (FIG. 3a). It was found that imidazole (24) was tolerated at the P1′ position (IC₅₀=0.96±0.09 μM), followed by isoxazole (25) (IC₅₀=2.47±0.27 μM), and oxazole (26) (IC₅₀=4.97±0.78 μM). Pyrazine (27) was tolerated at the P1 position (IC₅₀=4.93±0.79 μM); however, pyrimidine (29) and imidazole (30) were not preferred (IC₅₀>20 μM).

Next, the above identified favorable P1′, P2, P1, and P3/P4 substitutions were combined and the designed compounds were shown in FIG. 4e. Compounds 36, 37, and 38 were the most potent leads with IC₅₀ values of 0.81±0.24, 0.67±0.15, and 0.28±0.07 μM, respectively. Compound 39 and 40 were also highly active with IC₅₀ values of 1.48±0.56 and 1.05±0.09 μM, respectively.

Among the active compounds with IC₅₀ value lower than 5 μM, compounds 3, 6, 14, 17, 18, 26, 34, and 36 had moderate to high cellular cytotoxicity in Vero E6 cells (FIGS. 4a-4e red), while compounds 5, 20, 21, 23, 24, 25, 27, 31, 32, 37, 38, 39, and 40 were well tolerated and the CC₅₀ values were greater than 100 μM.

Next, compounds with potent enzymatic inhibition (IC₅₀≤1 μM) and low cellular cytotoxicity (CC₅₀>100 μM) were prioritized for the cellular antiviral assay with infectious SARS-CoV-2 in Vero E6 cells using the immunofluorescence assay as the primary assay (Table 2). ML188 (1) was included as a control. It was found that ML188 (1) was inactive in the antiviral assay (EC₅₀>20 μM), probably due to its incomplete inhibition of the M^{pro} in the cellular content. Gratifyingly, compounds 20, 23, 24, 37, 38, and 40 all had potent cellular antiviral activity with EC₅₀ values ranging from 0.82 to 4.54 μM.

TABLE 2
Antiviral activity and selectivity index of non-covalent SARS-CoV-2 M^{pro}
inhibitors. (Selection criteria IC₅₀ < 1 μM, CC₅₀ > 100 μM).

| | | | SARS- | |
|---|---|---|---|---|
| | SARS- | | CoV-2 | |
| | CoV-2 | Vero E6 | antiviral | Selectivity |
| Compound | M^{pro} | Cytotoxicity | assay | index SI |
| ID | IC₅₀ (μM) | CC₅₀ (μM) | EC₅₀ (μM) | CC₅₀/EC₅₀ |
| 1 ML188 | 10.96 ± 1.58 | >125 | >20 | N.A. |
| 20 Jun8-76-5 | 0.94 ± 0.33 | >200 | 1.73 ± 0.19 | >115.6 |
| 23 Jun8-76-3 | 0.31 ± 0.13 | >200 | 1.61 ± 0.29 | >124.2 |
| 24 Jun8-90-3 | 0.96 ± 0.09 | 129.38 ± 25.12 | | |
| 37 Jun9-5-2 | 0.67 ± 0.15 | 147.8 ± 13.9 | 4.54 ± 0.69 | 32.6 |
| 38 Jun9-4-4 | 0.28 ± 0.07 | >200 | 0.82 ± 0.56 | >243.9 |
| 40 Jun9-12-2 | 1.05 ± 0.09 | >200 | 2.04 ± 1.08 | >98.0 |

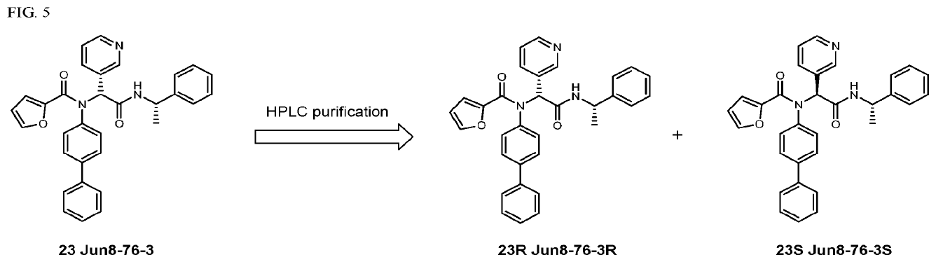

Given the potent antiviral activity and a high selectivity index of these potent lead compounds, we then selected compound 23 (Jun8-76-3) for further characterization. The two diasteromers of 23 (Jun8-76-3) were separated by reverse phase HPLC (FIG. 5). Both diasteromers were tested in the FRET-based enzymatic assay. GC376 was included as a positive control. It was found that the 23R Jun8-76-3R is the most potent diasteromer with an IC₅₀ value of 0.15±0.03 μM, while the 23S diasteromer was inactive (IC₅₀>10 μM) (Table 2). The stereochemistry of 23R (Jun8-76-3R) was determined by the co-crystal structure with SARS-CoV-2 M^{pro} as described in the following section. Compared with the parent compound ML188 (1), the optimized lead 23R had more than 73-fold increase in enzymatic inhibition against SARS-CoV-2 M^{pro}. Compound 23R also showed comparable potency against SARS-CoV M^{pro} with an IC₅₀ value of 0.27±0.03 μM. Both ML188 (1) and 23R did not inhibit the SARS-CoV-2 papain-like protease (PL^{pro}) (IC₅₀>20 μM) (Table 3), suggesting the inhibition of SARS-CoV-2 M^{pro} by 23R is specific.

TABLE 3
Enzymatic inhibition, antiviral activity and selectivity index of 23a.

| | | | | | SARS-CoV-2 | |
|---|---|---|---|---|---|---|
| | SARS-CoV-2 | SARS-CoV | SARS-CoV-2 | Vero E6 | antiviral | Selectivity |
| Compound | M^{pro} | M^{pro} | PL^{pro} | Cytotoxicity | assay | index SI |
| ID | IC₅₀ (μM) | IC₅₀ (μM) | IC₅₀ (μM) | CC₅₀ (μM) | EC₅₀ (μM) | CC₅₀/EC₅₀ |
| 1 ML188 | 10.96 ± 1.58 | 11.23 ± 1.61 | >60 | >125 | >20 | N.A. |
| GC376 | 0.033 ± 0.003 | 0.035 ± 0.002 | >60 | >125 | | |
| 23R Jun8- | 0.15 ± 0.03 | 0.27 ± 0.03 | >60 | | | |
| 76-3R | | | | | | |
| 23S Jun8- | >10 | N.T. | N.T. | | | |
| 76-3S | | | | | | |

### Example III. Binding of 23R (Jun8-76-3R) to SARS-CoV-2 M^{pro} in the Thermal Shift Binding Assay and Native Mass Spectrometry Binding Assay

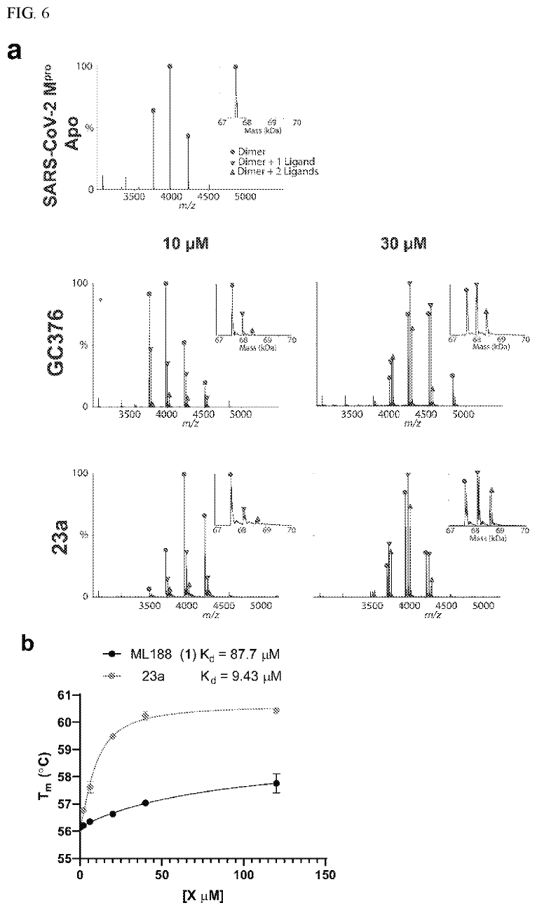

The binding of compound 23R to SARS-CoV-2 M^{pro} was characterized in the native mass spectrometry binding assay and the thermal shift binding assay (FIG. 6). In the native mass spectrometry binding assay, compound 23R showed dose-dependent binding to SARS-CoV-2 M^{pro}, similar to the positive control GC376, with a binding stoichiometry of one drug per monomer (FIG. 6a). Similarly, compound 23R also showed dose-dependent stabilization of the SARS-CoV-2 M^{pro} in the thermal shift binding assay with a K_{d} value of 9.43 μM, a 9.3-fold increase compared to ML188 (1) (FIG. 6b).

### Example IV. X-Ray Crystal Structure of SARS-CoV-2 M^{pro} with 23R (Jun8-76-3A)

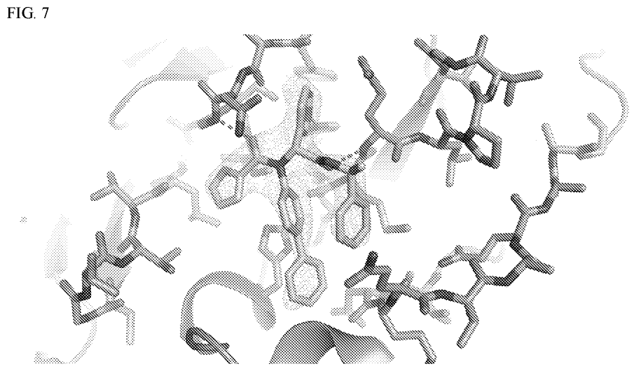

An X-ray crystal structure of SARS-CoV-2 M^{pro} with 23R (Jun8-76-3R) was obtained. FIG. 7 depicts an X-ray crystal structure of SARS-CoV-2 M^{pro} in complex with 23R.

### Example V. Materials and Methods for Examples I-IV

Cell lines and viruses. Human rhabdomyosarcoma (RD), MDCK, Vero, Huh-7, and HCT-8 cell lines were maintained in Dulbecco's modified Eagle's medium (DMEM) medium; Caco-2 and MRC-5 cell lines were maintained in Eagle's Minimum Essential Medium (EMEM) medium. Both media were supplemented with 10% fetal bovine serum (FBS) and 1% penicillin-streptomycin antibiotics. Cells were kept at 37° C. in a 5% CO₂ atmosphere. VERO E6 cells (ATCC, CRL-1586) were cultured in Dulbecco's modified Eagle's medium (DMEM), supplemented with 5% heat inactivated FBS in a 37° C. incubator with 5% CO₂. SARS-CoV-2, isolate USA-WA1/2020 (NR-52281), was obtained through BEI Resources and propagated once on VERO E6 cells before it was used for this study. Studies involving the SARS-CoV-2 were performed at the UTHSCSA biosafety level-3 laboratory by personnel wearing powered air purifying respirators.

Protein expression and purification. SARS CoV-2 main protease (M^{pro} or 3CL) gene from strain BetaCoV/Wuhan/WIV04/2019 was ordered from GenScript (Piscataway, NJ) in the pET29a(+) vector with E. coli codon optimization. The expression and purification of His-tagged SARS CoV-2 M^{pro} (M^{pro}-His) was described as previously.³ Briefly, the M^{pro} gene was inserted into pET29a(+) with NdeI/XhoI digestion sites. The N-terminal methionine was removed by E. Coli methionine aminopeptidase. There are extra LEHHHHHH (SEQ ID NO: 1) residues at the C-terminus. The protein sequence for the C-terminal His-tagged SARS-CoV-2 M^{pro} is:

(SEQ ID NO: 2)
SGFRKMAFPS GKVEGCMVQV TCGTTTLNGL WLDDVVYCPR
HVICTSEDML NPNYEDLLIR KSNHNFLVQA GNVQLRVIGH
SMQNCVLKLK VDTANPKTPKYKFVRIQPGQ TFSVLACYNG
SPSGVYQCAM RPNFTIKGSF LNGSCGSVGF NIDYDCVSFC
YMHHMELPTG VHAGTDLEGN FYGPFVDRQT
AQAAGTDTTITVNVLAWLYA AVINGDRWFL NRFTTTLNDF
NLVAMKYNYE PLTQDHVDIL GPLSAQTGIA VLDMCASLKE
LLQNGMNGRT ILGSALLEDE FTPFDVVRQSGVTFQLEHHHHHH.

For HM-M^{pro} expression and purification, the SARS-CoV-2 M^{pro} gene from strain BetaCoV/Wuhan/WIV04/2019 GenScript (Piscataway, NJ, USA) was inserted into pETGSTSUMO vector. The plasmid was transformed into Rosetta™(DE3) pLysS Competent Cells (Novagen). A single colony was picked for overnight growth to inoculate 50 mL of LB broth with 50 μg/mL kanamycin and 35 μg/mL chloramphenicol. 10 mL of the overnight culture was used to inoculate 1 L of LB broth with 50 μg/mL kanamycin and 35 μg/mL chloramphenicol. The 1 L culture was grown at 250 RPM, 37° C. until OD reached 0.6˜0.8. Expression was then induced with 0.5 mM IPTG at 250 RPM, 20° C. overnight. The culture was centrifuged at 5,000×g for 20 minutes and the resulting pellet was resuspended in 30 mL of the lysis buffer (20 mM Tris-HCl pH 8.4, 300 mM NaCl, 10% glycerol, and 20 mM imidazole). These cells were lysed by sonication on a 10 second sonication/15 second rest cycle for a total of 15 minutes at an amplitude of 6. The lysate was centrifuged at 40,000×g for 45 minutes at 4° C. and the supernatant was filtered, then loaded onto a HiTrap HP column. The column was washed with lysate buffer and the protein was then eluted by linear gradient of imidazole. The peak of the protein was pooled and concentrated. The protein was then diluted in ULP1 cleavage buffer (20 mM Tris pH 8.0, 100 mM NaCl and 10% glycerol). The protease ULP1 was added at 1:20 ratio with incubation at 20° C. overnight. The sample was loaded to a HisTrap HP column and the flow through containing the HM-M^{pro} was collected. The HM-M^{pro} was concentrated and loaded to Superdex 200/16 equilibrated with 20 mM Tris pH 8.0, 250 mM NaCl. The peak fractions were pooled and concentrated to 10 mg/mL and flash-frozen with liquid nitrogen. The purity of the protein was evaluated by SDS-PAGE. The protein sequence for the SARS-CoV-2 HM-M^{pro} is:

(SEQ ID NO: 3)
HMSGFRKMAFPS GKVEGCMVQV TCGTTTLNGL WLDDVVYCPR
HVICTSEDML NPNYEDLLIR KSNHNFLVQA GNVQLRVIGH
SMQNCVLKLK VDTANPKTPKYKFVRIQPGQ TFSVLACYNG
SPSGVYQCAM RPNFTIKGSF LNGSCGSVGF NIDYDCVSFC
YMHHMELPTG VHAGTDLEGN FYGPFVDRQT
AQAAGTDTTITVNVLAWLYA AVINGDRWFL NRFTTTLNDF
NLVAMKYNYE PLTQDHVDIL GPLSAQTGIA VLDMCASLKE
LLQNGMNGRT ILGSALLEDE FTPFDVVRQCSGVTFQ.

The expression and purification of SARS CoV-2 M^{pro} with unmodified N- and C-termini (M^{pro}). SARS CoV-2 M^{pro} gene was subcloned from pET29a(+) to pE-SUMO vector according to manufacturer's protocol (LifeSensors Inc, Malvern PA). The forward primer with BsaI site is: GCGGTCTCAAGGTTCAGGATTTAGGAAGATGGCATTTCC (SEQ ID NO: 4) ; the reverse primer with XbaI site is: GCTCTAGATTACTGAAAGGTCACGCCGCTGCATTGACG (SEQ ID NO: 5). After removal of SUMO tag with SUMO protease, there is no any extra residues at either N- or C-termini. pE-SUMO plasmid with SARS CoV-2 Main protease gene (M^{pro}) was transformed into BL21(DE3) cells with kanamycin selection. A single colony was picked to inoculate 10 ml LB media and was cultured 37° C. overnight. This 10 ml culture was added to 1 liter LB media and grown to around OD 600 of 0.8. This culture was cooled on ice for 15 min, then induced with 0.5 mM IPTG. Induced cultures were incubated at 18° C. for an additional 24 h and then harvested, lysed same way as His-tagged M^{pro} protein.³ The supernatant was incubated with Ni-NTA resin for overnight at 4° C. on a rotator. The Ni-NTA resin was thoroughly washed with 30 mM imidazole in wash buffer (50 mM Tris [pH 7.0], 150 mM NaCl, 2 mM DTT), SUMO-tagged M^{pro} was eluted from Ni-NTA with 300 mM imidazole. Eluted SUMO-tagged M^{pro} was dialyzed against 100-fold volume dialysis buffer (50 mM Tris [pH 7.0], 150 mM NaCl, 2 mM DTT) in a 10,000-molecular-weight-cutoff dialysis tubing. After dialysis, SUMO-tagged M^{pro} was incubated with SUMO protease 1 at 4° C. for overnight, and SUMO tag was removed by application of another round of Ni-NTA resin. The purity of the protein was confirmed with SDS-page gel. The protein sequence for the native SARS-CoV-2 M^{pro} is:

(SEQ ID NO: 6)
SGFRKMAFPS GKVEGCMVQV TCGTTTLNGL WLDDVVYCPR
HVICTSEDML NPNYEDLLIR KSNHNFLVQA GNVQLRVIGH
SMQNCVLKLK VDTANPKTPKYKFVRIQPGQ TFSVLACYNG
SPSGVYQCAM RPNFTIKGSF LNGSCGSVGF NIDYDCVSFC
YMHHMELPTG VHAGTDLEGN FYGPFVDRQT
AQAAGTDTTITVNVLAWLYA AVINGDRWFL NRFTTTLNDF
NLVAMKYNYE PLTQDHVDIL GPLSAQTGIA VLDMCASLKE
LLQNGMNGRT ILGSALLEDE FTPFDVVRQCSGVTFQ.

The expression and purification of SARS CoV-2 papain-like protease (PL^{pro}). SARS CoV-2 papain-like protease (PL^{pro}) gene (ORF lab 1564 to 1876) from strain BetaCoV/Wuhan/WIV04/2019 was ordered from GenScript (Piscataway, NJ) in the pET28b(+) vector with E. coli codon optimization. The SARS CoV-2 PL^{pro} gene was inserted into pET28b(+) with NcoI/XhoI sites. The expression and purification procedures are similar to that of M^{pro}-His protein as described above, except that the lysis buffer and Ni-NTA wash and elution buffer are in pH7.5 (50 mM Tris [pH 7.5], 150 mM NaCl, 2 mM DTT). The N-terminal methionine was removed by E. coli methionine aminopeptidase based on our mass spectrum result. There are extra LEHHHHHH residues at C-terminal. The final protein sequence for the SARS-CoV-2 PL^{pro} is:

(SEQ ID NO: 7)
GEVRTIKVFTTVDNINLHTQVVDMSMTYGQQFGPTYLDGADVTKIKPHNS
HEGKTFYVLPNDDTLRVEAFEYYHTTDPSFLGRYMSALNHTKKWKYPQVN
GLTSIKWADNNCYLATALLTLQQIELKFNPPALQDAYYRARAGEAANFCA
LILAYCNKTVGELGDVRETMSYLFQHANLDSCKRVLNVVCKTCGQQQTTL
KGVEAVMYMGTLSYEQFKKGVQIPCTCGKQATKYLVQQESPFVMMSAPPA
QYELKHGTFTCASEYTGNYQCGHYKHITSKETLYCIDGALLTKSSEYKGP
ITDVFYKENSYTTTIKLEHHHHHH.

Human liver Cathepsin L was purchased from EMD Millipore (Cat # 219402).

Peptide synthesis. The SARS-CoV-2 M^{pro} FRET substrate Dabcyl-KTSAVLQ/SGFRKME(Edans) (SEQ ID NO: 8) was synthesized as described before.³ The SARS-CoV-2 PL^{pro} FRET substrate Dabcyl-FTLRGG/APTKV(Edans) (SEQ ID NO: 9) was synthesized by solid-phase synthesis through iterative cycles of coupling and deprotection using the previously optimized procedure.⁹ Specifically, chemmatrix rink-amide resin was used. Typical coupling condition was 5 equiv of amino acid, 5 equiv of HATU, and 10 equiv of DIEA in DMF for 5 minutes at 80° C. For deprotection, 5% piperazine plus 0.1 M HOBt were used and the mixture was heated at 80° C. for 5 minutes. The peptide was cleaved from the resin using 95% TFA, 2.5% Tris, 2.5% H₂O and the crude peptide was precipitated from ether after removal of TFA. The final peptide was purified by preparative HPLC. The purify and identify of the peptide were confirmed by analytical HPLC (>98% purity) and mass spectrometry. [M+2]²⁺ calculated 888.04, detected 888.80.

Compound synthesis and characterization. Details for the synthesis procedure (Scheme S1) and characterization for compounds UAWJ257, UAWJ246, UAWJ247, and UAWJ248 can be found in the supplementary information.

Native Mass Spectrometry. Prior to analysis, the protein was buffer exchanged into 0.2 M ammonium acetate (pH 6.8) and diluted to 10 μM. DTT was dissolved in water and prepared at a 400 mM stock. Each ligand was dissolved in ethanol and diluted to 10× stock concentrations. The final mixture was prepared by adding 4 μL protein, 0.5 μL DTT stock, and 0.5 μL ligand stock for final concentration of 4 mM DTT and 8 μM protein. Final ligand concentrations were 10 μM. The mixtures were then incubated for 10 minutes at room temperature prior to analysis. Each sample was mixed and analyzed in triplicate. Native mass spectrometry (MS) was performed using a Q-Exactive HF quadrupole-Orbitrap mass spectrometer with the Ultra-High Mass Range research modifications (Thermo Fisher Scientific). Samples were ionized using nano-electrospray ionization in positive ion mode using 1.0 kV capillary voltage at a 150° C. capillary temperature. The samples were all analyzed with a 1,000-25,000 m/z range, the resolution set to 30,000, and a trapping gas pressure set to 3. Between 10 and 50 V of source fragmentation was applied to all samples to aid in desolvation. Data were deconvolved and analyzed with UniDec.¹⁰

Enzymatic assays. The main protease (M^{pro}) enzymatic assays were carried out exact as previously described in pH 6.5 reaction buffer containing 20 mM HEPES pH 6.5, 120 mM NaCl, 0.4 mM EDTA, 20% glycerol and 4 mM DTT.³

The SARS-CoV-2 papain-like protease (PL^{pro}) enzymatic assays were carried out as follows: the assay was assembled in 96-well plates with 100 μl of 200 nM PL^{Pro} protein in PL^{Pro} reaction buffer (50 mM HEPES, pH7.5, 0.01% triton-100 and 5 mM DTT). Then 1 μl testing compound at various concentrations was added to each well and incubated at 30° C. for 30 min. The enzymatic reaction was initiated by adding 1 μl of 1 mM FRET substrate (the final substrate concentration is 10 μM). The reaction was monitored in a Cytation 5 image reader with filters for excitation at 360/40 nm and emission at 460/40 nm at 30° C. for 1 hr. The initial velocity of the enzymatic reaction with and without testing compounds was calculated by linear regression for the first 15 min of the kinetic progress curve. The IC₅₀ values were calculated by plotting the initial velocity against various concentrations of testing compounds with a dose response function in Prism 8 software.

The cathepsin L enzymatic assay was carried out as follows: human liver cathepsin L (EMD Millipore 219402) was activated by incubating at reaction buffer (20 mM sodium acetate, 1 mM EDTA and 5 mM DTT pH5.5) for 30 min at 30° C. Upon activation, the assay was assembled in 96-well plates with 100 μl of 300 pM cathepsin L protein in cathepsin L reaction buffer. Then 1 μl testing compound at various concentrations was added to each well and incubated at 30° C. for 30 min. The enzymatic reaction was initiated by adding 1 μl of 100 μM FRET substrate Z-Phe-Arg-AMC (the final substrate concentration is about 1 μM). The reaction was monitored in a Cytation 5 image reader with filters for excitation at 360/40 nm and emission at 460/40 nm at 30° C. for 1 hr. The IC₅₀ values were calculated as described in above section.

Differential scanning fluorimetry (DSF). The thermal shift binding assay (TSA) was carried out using a Thermal Fisher QuantStudio™ 5 Real-Time PCR System as described previously.³ Briefly, 3 μM SARS-CoV-2 M^{pro} protein in M^{pro} reaction buffer (20 mM HEPES, pH 6.5, 120 mM NaCl, 0.4 mM EDTA, 4 mM DTT and 20% glycerol) was incubated with testing compounds at 30° C. for 30 min. 1× SYPRO orange dye was added and fluorescence of the well was monitored under a temperature gradient range from 20° C. to 90° C. with 0.05° C./s incremental step.

Cytotoxicity measurement. Evaluation of the cytotoxicity of compounds were carried out using the neutral red uptake assay.¹¹ Briefly, 80,000 cells/mL of the tested cell lines were dispensed into 96-well cell culture plates at 100 μL/well. Twenty-four hours later, the growth medium was removed and washed with 150 μL PBS buffer. 200 μL fresh serum-free medium containing serial diluted compounds was added to each well. After incubating for 5 days at 37° C., the medium was removed and replaced with 100 μL DMEM medium containing 40 μg/mL neutral red and incubated for 2-4 h at 37° C. The amount of neutral red taken up was determined by measuring the absorbance at 540 nm using a Multiskan FC Microplate Photometer (Fisher Scientific). The CC₅₀ values were calculated from best-fit dose response curves with variable slope in Prism 8.

Immunofluorescence assay. Vero E6 cells in 96-well plates (Corning) were infected with SARS-CoV-2 (USA-WA1/2020 isolate) at MOI of 0.05 in DMEM supplemented with 1% FBS. Immediately before the viral inoculation, the tested compounds in a three-fold dilution concentration series were also added to the wells in triplicate. The infection proceeded for 48 h without the removal of the viruses or the compounds. The cells were then fixed with 4% paraformaldehyde, permeabilized with 0.1% Triton-100, blocked with DMEM containing 10% FBS, and stained with a rabbit monoclonal antibody against SARS-CoV-2 NP (GeneTex, GTX635679) and an Alexa Fluor 488-conjugated goat anti-mouse secondary antibody (ThermoFisher Scientific). Hoechst 33342 was added in the final step to counterstain the nuclei. Fluorescence images of approximately ten thousand cells were acquired per well with a 10× objective in a Cytation 5 (BioTek). The total number of cells, as indicated by the nuclei staining, and the fraction of the infected cells, as indicated by the NP staining, were quantified with the cellular analysis module of the Gen5 software (BioTek).

Plaque assay. Vero E6 cells in 6-well plates (Corning) were infected with SARS-CoV-2 (USA-WA1/2020 isolate) at approximately 40 PFU per well. After 1 hour of incubation at 37° C., the inoculum was removed and replaced with DMEM containing 1% FBS, 1.2% Avicel RC-591 (Dupont) and the tested compounds at different concentrations, in duplicate. After 3 days of infection, the overlay was removed, and the cells were fixed with 4% paraformaldehyde and stained with 0.2% crystal violet.

M^{pro} crystallization and structure determination. SARS-CoV-2 M^{pro} was diluted to 5 mg/mL and incubated with 1.5 mM of inhibitor at 4° C. overnight. Samples were centrifuged at 13,000×g for 1 minute to remove precipitate. Crystals were grown by mixing the protein-inhibitor sample with an equal volume of crystallization buffer (20% PEG 3000, 0.2 M Na Citrate pH 5.6) in a vapor diffusion, hanging drop apparatus. A cryoprotectant solution of 35% PEG 3000 and 30% glycerol was added directly to the drop and soaked for 15 minutes. Crystals were then flash frozen in liquid nitrogen for X-ray diffraction.

X-ray diffraction data for the SARS-CoV-2 M^{pro} structures were collected on the SBC 19-ID beamline at the Advanced Photon Source (APS) in Argonne, IL, and processed with the HKL3000 software suite.¹² The CCP4 versions of MOLREP was used for molecular replacement using a previously solved SARS-CoV-2 M^{pro} structure, PDB ID: 7BRR as a reference model for the dimeric P2₁ M^{pro} with UAWJ246. ^{13,14} PDB ID 6YB7 was used as the reference model for the C2 monomeric M^{pro} with calpain inhibitors II/XII and UAWJ247, and the P1 dimeric structure with UAWJ248.¹⁵ PDB ID 6WTT was used as the reference model for the P3₂21 trimer with UAWJ246.¹⁶ Rigid and restrained refinements were performed using REFMAC and model building was performed with COOT.^{17,18} Protein structure figures were made using PyMOL (Schrodinger, LLC).

### Example VI

This example describes the discovery of di- and trihaloacetamides as covalent SARS-CoV-2 main protease inhibitors with high target specificity.

The ongoing COVID-19 pandemic is a timely reminder that direct-acting antivirals are urgently needed. Despite the groundbreaking success of mRNA vaccines, SARS-CoV-2 is likely to remain a significant public health concern in the foreseeable future for several reasons. First, variant viruses with escape mutations continue to emerge, which compromise the efficacy of vaccines (see, Harvey, W. T.; et al., Nat. Rev. Microbiol. 2021, 19 (7), 409-424). Second, a portion of the population opt out of vaccination based on their religious beliefs, concerns of long-term side effects or other reasons. As such, it is unpredictable when or whether herd immunity can be achieved. Third, the durability of COVID vaccines is currently unknown. Therefore, antivirals are important complements of vaccines to combat not only the current COVID-19 pandemic but also future outbreaks.

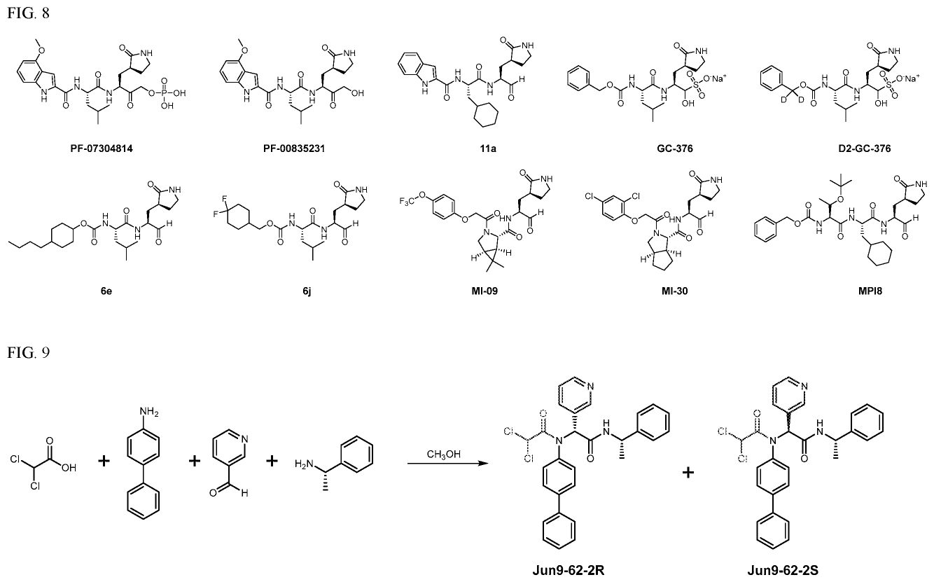

In combating the COVID-19 pandemic, researchers around the globe are racing to discover countermeasures. Drug repurposing led to the identification of remdesivir as the first FDA-approved SARS-CoV-2 antiviral. EIDD-2801, another viral polymerase inhibitor discovered through a similar approach, is in human clinical II/III trials (see, Cox, R. M.; et al., Nat. Microbiol. 2021, 6 (1), 11-18). Among the list of drug targets being pursued for antiviral development, the viral polymerase including the main protease (M^{pro}) and the papain-like protease (PL^{pro}) are the most extensively studied (see, Morse, J. S.; et al., Chembiochem 2020, 21 (5), 730-738). The main protease is a cysteine protease and is responsible for digesting the viral polyprotein during the viral replication. M^{pro} functions as a dimer and has a high substrate preference of glutamine at the P1 position. M^{pro} is a validated antiviral drug target and M^{pro} inhibitors have shown antiviral activity in in vitro cell cultures and in vivo animal models (FIG. 8) (see, Qiao, J.; et la., Science 2021, 371 (6536), 1374-1378; Dampalla, C. S.; et al., Proc. Natl. Acad. Sci. U. S. A. 2021, 118 (29), e2101555118; Caceres, C. J.; et al., Sci. Rep. 2021, 11 (1), 9609; Ma, C.; et al., Cell Res. 2020, 30 (8), 678-692; Sacco, M. D.; et al., Sci. Adv. 2020, 6 (50), eabe0751). Two Pfizer M^{pro} inhibitors PF-07304814 and PF-07321332 are advanced to phase I clinical trial (see, Owen, D. R.; et al., medRxiv 2021, 2021.07.28; Boras, B.; et al., bioRxiv 2020, 2020.09.12). Additional promising leads are listed in Table 4, which are in different stages of preclinical development. The success of fast-track development of SARS-CoV-2 M^{pro} inhibitors is a result of accumulated expertise and knowledge in targeting SARS-CoV M^{pro} and similar picornavirus 3C-like (3CL) proteases over the years (see, Ghosh, A. K.; et al., ChemMedChem 2020, 15 (11), 907-932). Despite the tremendous progress in developing M^{pro} inhibitors, the selectivity profiling has thus far been largely neglected. It is essential to address the target selectivity issue at the early stage of drug discovery to avoid catastrophic failures in the later clinical studies. No cysteine protease inhibitor has received FDA approval so far, and the lack of target specificity might be one of the major reasons.

The majority of current reported SARS-CoV-2 M^{pro} inhibitors are peptidomimetic covalent inhibitors with a reactive warhead such as ketone, aldehyde or ketoamide (see, Ghosh, A. K.; et al., ChemMedChem 2020, 15 (11), 907-932). Some of the promising examples include the Pfizer compounds PF-07304814 (the parent compound PF-00835231) (see, Boras, B.; et al., bioRxiv 2020, 2020 Sep. 12), 11a (see, Dai, W.; et al., Science 2020, 368 (6497), 1331-1335), GC-376 (see, Vuong, W.; et al., Nat. Commun. 2020, 11 (1), 4282; Ma, C.; et al., Cell Res. 2020, 30 (8), 678-692), the deuterated GC-376 (D2-GC-376) (see, Dampalla, C. S.; et al., Proc. Natl. Acad. Sci. U. S. A. 2021, 118 (29)), 6e, 6j (see, Rathnayake, A. D.; et al., Sci. Transl. Med. 2020, 12 (557)), MI-09, MI-30 (see, Qiao, J.; et al., Science 2021, 371 (6536), 1374-1378),⁴ and MPI8 (see, Yang, K. S.; et al., ChemMedChem 2021, 16 (6), 942-948) (FIG. 8). Although the high reactivity of these reactive warheads, especially the aldehyde, confers potent enzymatic inhibition and cellular antiviral activity, it inevitably leads to off-target side effects through reacting with some host proteins (see, Ma, X. R.; et al., ChemMedChem 2021, doi: 10.1002/cmdc.202100456; Steuten, K.; et al., ACS Infect. Dis. 2021, 7 (6), 1457-1468; Xia, Z.; et al., ACS Pharmcol. Transl. Sci. 2021, 4 (4), 1408-1421; Vandyck, K.; et al., Biochem. Biophys. Res. Commun. 2021, 555, 134-139). For example, we and others have shown that GC-376 is a potent inhibitor of cathepsin L (Table 4) (see, Steuten, K.; et al., ACS Infect. Dis. 2021, 7 (6), 1457-1468; Hu, Y.; et al., ACS Infect. Dis. 2021, 7 (3), 586-597). A recent study revealed that MP18, an analog of GC-376 with an aldehyde warhead, inhibits cathepsins B, L, and K with IC₅₀ values of 1.2, 230, and 180 nM, respectively (see, Yang, K. S.; et al., ChemMedChem 2021, 16 (6), 942-948). The off-target effect is also a potential concern for some of the most advanced M^{pro} inhibitors including the clinical candidate PF-07304814 (see, Hoffman, R. L.; et al., J. Med. Chem. 2020, 63 (21), 12725-12747), compounds 6j and 6e which showed in vivo antiviral efficacy against MERS-CoV-2 infection in mice (see, Rathnayake, A. D.; et al., Sc. Transl. Med. 2020, 12 (557), eabc5332), and compound 11a with potent in vitro antiviral activity (Table 4) (see, Dai, W.; et al., Science 2020, 368 (6497), 1331-1335). All of these compounds are potent inhibitors of cathepsin L. The lack of target specificity might arise from the high reactivity of the aldehyde warhead, and the design of covalent inhibitors with a high target specificity remains a daunting task.

TABLE 4
Target specificity of SARS-CoV-2 M^{pro} inhibitors.

| | SARS-CoV-2 M^{pro} | Cathepsin L | | |
|---|---|---|---|---|
| Compound | IC₅₀ (nM) | IC₅₀ (nM) | Additional off targets | References |
| GC-376 | 33 | 0.99 | Calpain I (IC₅₀ = 74 nM) | 7, 16-17, 21-22 |
| | | | Cathepsin K (IC₅₀ = 0.56 nM) | |
| MPI8 | 105 | 1.2 | Cathepsin B (IC₅₀ = 230 nM) | 13, 23 |
| | | | Cathepsin K (IC₅₀ = 180 nM) | |
| PF-00835231 | 5 | 146 | Cathepsin B (IC₅₀ = 1.3 μM) | 18, 24 |
| 6e | 10 | <0.5 | — | 19, 24 |
| 6j | 7 | <0.5 | — | 19, 24 |
| 11a | 8 | 0.21 | — | 20, 24 |

In experiments conducted during the course of developing embodiments for the present invention, we report the rational design of covalent M^{pro} inhibitors with novel cysteine reactive warheads and high target specificity. Specifically, guided by the X-ray crystal structure of SARS-CoV-2 M^{pro} with 23R (Jun8-76-3A) (PDB: 7KX5), one of the most potent non-covalent M^{pro} inhibitors reported thus far (see, Kitamura, N.; et al., J. Med. Chem. 2021, doi: 10.1021/acs.jmedchem.1c00509), we systematically explored a number of novel electrophiles in replacement of the P1′ furyl substitution in 23R. The aim was to identify C145 reactive electrophiles with both potent M^{pro} inhibition and high target selectivity. This effort led to the discovery of several novel cysteine reactive warheads including dichloroacetamide, dibromoacetamide, tribromoacetamide, 2-bromo-2, 2-dichloroacetamide, and 2-chloro-2, 2-dibromoacetamide. One of the most potent lead compounds Jun9-62-2R (dichloroacetamide) inhibited SARS-CoV-2 M^{pro} and viral replication with an IC₅₀ of 0.43 μM of and an EC₅₀ of 2.05 μM in Caco2-hACE2 cells. Significantly, unlike GC-376, Jun9-62-2R (dichloroacetamide) and Jun9-88-6R (tribromoacetamide) were highly selective toward M^{pro} and did not inhibit the host proteases including calpain I, cathepsins B, K, L, caspase-3, and trypsin. X-ray crystal structure of SARS-CoV-2 M^{pro} with Jun9-62-2R (dichloroacetamide) and Jun9-57-3R (chloroacetamide) revealed that the C145 forms a covalent bond with the reactive warheads. Overall, the discovery of these di- and tri-haloacetamides as novel cysteine reactive warheads shed light on feasibility of developing SARS-CoV-2 M^{pro} inhibitors with high target specificity and cellular selectivity index. These novel compounds represent valuable chemical probes for target validation and further development as SARS-CoV-2 antivirals.

### Synthesis of Covalent M^{pro} Inhibitors

All designed compounds were synthesized by the one-pot Ugi four-component reaction (Ugi-4CR) as shown for Jun9-62-2 (FIG. 9) with yields from 33% to 88%. For compounds with potent enzymatic inhibition, the diastereomers were subsequently separated by chiral HPLC. The absolute stereochemistry of Jun9-57-3R and Jun9-62-2R was determined by X-ray crystallography, and the stereochemistry for the diastereomers of Jun9-90-4, Jun9-89-2, Jun9-89-4, and Jun9-88-6 were tentatively assigned based on their relevant retention time in chiral HPLC.

### Rational Design of Covalent M^{pro} Inhibitors

23R was designed based on the superimposed X-ray crystal structure of GC-376 with ML188 and UAWJ254 (see, Kitamura, N.; et al., J. Med. Chem. 2021, doi: 10.1021/acs.jmedchem.1c00509; Jacobs, J.; et al., J. Med. Chem. 2013, 56 (2), 534-546). The X-ray crystal structure showed that the furyl substitution at the P1′ position of 23R is in close proximity with the catalytic cysteine 145 (3.4 Å between C145 sulfur and the C-2 carbon of furyl, PDB: 7KX5) (FIG. 10A), suggesting replacement of furyl with a reactive warhead might lead to covalent inhibitors (FIG. 10B). 23R is an ideal lead candidate for the design of covalent M^{pro} inhibitors for several reasons: 1) the P1, P2, and P3 substitutions have already been optimized; 2) the designed compounds can be expeditiously synthesized by the one-pot Ugi-4CR; and 3) a diverse of cysteine reactive warheads are commercially available and can be promptly introduced at the P1′ position to react with the C145.

Although a number of thiol-reactive warheads have been exploited in the design of covalent protease and kinase inhibitors (see, Abdeldayem, A.; et al., Chem. Soc. Rev. 2020, 49 (9), 2617-2687; Siklos, M.; et al., Acta Pharm. Sin. B 2015, 5 (6), 506-519; Cianni, L.; et al., J. Med. Chem. 2019, 62 (23), 10497-10525, we decided to focus on pharmacologically compliant reactive warheads from the FDA-approved drugs. The majority of FDA-approved thiol-reactive drugs are kinase inhibitors including ibrutinib, osimertinib, zanubrutinib, acalabrutinib, dacomitinib, neratinib, and afatinib (FIG. 10C) (see, Abdeldayem, A.; et al., Chem. Soc. Rev. 2020, 49 (9), 2617-2687). As such, acrylamide and 2-butynamide were chosen as reactive warheads in our initial design of covalent SARS-CoV-2 M^{pro} inhibitors (FIG. 10B). Chloroacetamide was also chosen as it was previously explored by Pfizer for the development of SARS-CoV and SARS-CoV-2 M^{pro} inhibitors (Pfizer compound 12) (FIG. 10C) (see, Hoffman, R. L.; et al., J. Med. Chem. 2020, 63 (21), 12725-12747). Chloroacetamide is frequently used as a reactive warhead for designing chemical probes for target pull down (see, Hoch, D. G.; et al., Chem. Commun. 2018, 54 (36), 4501-4512). Finally, we included azidomethylene as it was previously shown to be a relatively unreactive cysteine warhed (see, Le, G. T.; et al., J. Am. Chem. Soc. 2006, 128 (38), 12396-12397; Yang, P.-Y.; et al., Org. Lett. 2008, 10 (10), 1881-1884). The fluoroacetamide was included as a control.

The designed covalent SARS-CoV-2 M^{pro} inhibitors were shown in FIG. 10D. All compounds were first tested in the FRET-based M^{pro} enzymatic assay. Active hits were further tested for cellular cytotoxicity to prioritize candidates for the following antiviral assay against SARS-CoV-2. It was found that the azidoacetamide Jun9-61-1 and the fluoracetamide Jun9-61-4 were not active (IC₅₀>20 μM). Surprisingly, the acrylamides Jun10-15-2 and Jun9-51-3 were also not active (IC₅₀>20 μM), suggesting the acrylamide might not be positioned at the right geometry for reacting with the C145. Gratifyingly, Jun9-62-1 with the 2-butynamide warhead showed potent inhibition with an IC₅₀ of 1.15 μM. However, Jun9-62-1 also had moderate cytotoxicity in both Vero E6 (CC₅₀=17.99 μM) and Calu-3 (CC₅₀=47.77 μM) cells. Similarly, covalent inhibitors with the chloroacetamide reactive warhead were potent inhibitors of SARS-CoV-2 M^{pro}. The most potent compound Jun9-57-3R inhibited SARS-CoV-2 M^{pro} with an IC₅₀ of 0.05 μM, comparable to the potency of GC-376 (IC₅₀=0.03 μM). Interestingly, the diastereomer Jun9-57-3S was also a potent M^{pro} inhibitor with an IC₅₀ of 1.13 μM. However, covalent inhibitors with the chloroacetamide warhead Jun9-54-1, Jun9-59-1, Jun9-55-2, Jun9-57-3R, Jun9-57-3S, Jun9-57-2, and Jun9-55-1 were highly cytotoxic in Vero E6 (CC₅₀<11 μM) and Calu-3 (CC₅₀<2 μM) cells, possibly due to their off-target effects on host proteins/DNAs. The low cellular selectivity index precludes further development of these covalent M^{pro} inhibitors as SARS-CoV-2 antivirals.

### Exploring Acrylamides and Haloacetamides as Novel Warheads for SARS-CoV-2 M^{pro} C145

For the acrylamide series of compounds, Jun9-72-3 and Jun10-31-4, both containing a 2-substituted acrylamide warhead, were not active against M^{pro} (IC₅₀>20 μM) (FIG. 11). However, compound Jun10-38-2 with the 2-chloroacrylamide showed potent inhibition against M^{pro} with an IC₅₀ of 4.22 μM.

For the haloacetamide series of compounds, the reference compound Jun9-54-1 with the classical chloroacetamide reactive warhead had potent inhibition against SARS-CoV-2 M^{pro} with an IC₅₀ of 0.17 μM. However, it was cytotoxic in both Vero E6 cells and Calu-3 cells with CC₅₀ values less than 3.5 μM. To increase the cellular selectivity index, we reasoned that substituted chloroacetamides or haloacetamides might have reduced cellular cytotoxicity while maintaining potent M^{pro} inhibition. It was found that Jun9-77-1 with the 2-chloropropanamide warhead was not active (IC₅₀>20 μM). Encouragingly, compound Jun9-62-2R with the dichloroacetamide warhead had potent inhibition against M^{pro} with an IC₅₀ of 0.43 μM while being non-cytotoxic to Vero E6 cells (CC₅₀>100 μM). In comparison, the corresponding diastereomer Jun9-62-2S was not active (IC₅₀>20 μM), which is consistent with the predicted binding mode (FIG. 10A). Given these promising results, we further designed two additional dichloroacetamide compounds Jun9-90-3 and Jun9-90-4 with variations at the P3/P4 substitutions. Similar to Jun9-62-2R, both Jun9-90-3R and Jun9-90-4R had potent inhibition against M^{pro} with IC₅₀ values of 0.30 and 0.46 μM, respectively. Both compounds were also non-cytotoxic to Vero E6 cells (CC₅₀>100 μM). In contrast, the corresponding diastereomers Jun9-90-3S and Jun9-90-4S were not active (IC₅₀>20 μM).

We further explored di- and trisubstituted haloacetamides as M^{pro} C145 reactive warheads (FIG. 11). Jun9-89-2R with the dibromoacetamide warhead is highly active with an IC₅₀ of 0.08 μM, however, the cell cytotoxicity also increased (CC₅₀=8.94 μM). The diastereomer Jun9-89-2S also had potent inhibition against M^{pro} with an IC₅₀ of 2.44 μM and comparable cytotoxicity (CC₅₀=4.57 μM). Jun9-76-4 with the 2, 2-dichloropropanamide warhead, Jun9-72-4 with the trichloroacetamide, Jun9-77-2 with the 2-chloro-2, 2-difluoroacetamide were all inactive against M^{pro} (IC₅₀>20 μM). Jun9-89-3 with the 2-bromo-2, 2-dichloroacetamide showed potent inhibition with an IC₅₀ of 1.20 μM. The cytotoxicity of Jun9-89-3 also improved (CC₅₀=32.43 μM). Jun9-89-4R with the 2-chloro-2, 2-dibromoacetamide warhead is highly potent with an IC₅₀ of 0.05 μM, but it was cytotoxic in Vero E6 cells (CC₅₀=8.41 μM). The diastereomer Jun9-89-4S was less active (IC₅₀=9.04 μM). Jun9-88-6R with the tribromoacetamide warhead had high potency against M^{pro} with an IC₅₀ of 0.08 μM, while the diastereomer Jun9-88-6S was less active (IC₅₀=7.16 μM). Both Jun9-88-6R and Jun9-88-6S had comparable cytotoxicity as Jun9-54-1 with CC₅₀ value of 5.48 and 5.99 μM, respectively.

### Pharmacological Characterization of SARS-CoV-2 M^{pro} Inhibitors with Novel Reactive Warheads

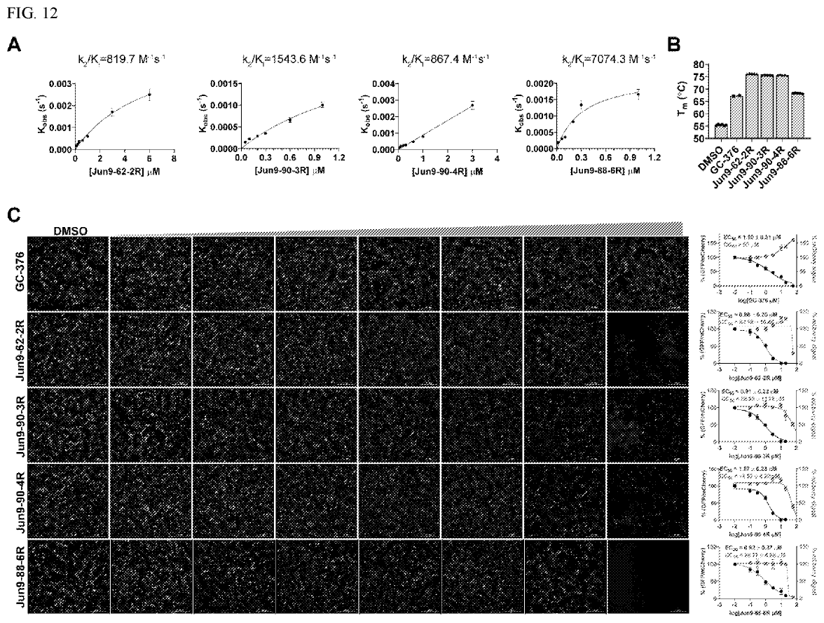

Based on the M^{pro} inhibition and cell cytotoxicity, four compounds were selected for mechanistic studies including Jun9-62-2R, Jun9-90-3R, Jun9-90-4R, and Jun9-88-6R (FIG. 12). Enzymatic kinetic studies suggested that these four compounds bind to M^{pro} in a two-step process: the first step reversible binding (K_{I}) and the second step irreversible binding (k_{inact}). The calculated k_{inact}/K_{I} values for Jun9-62-2R, Jun9-90-3R, Jun9-90-4R, and Jun9-88-6R were 819.7, 1543.6, 867.4, and 7074.3 M⁻¹ s⁻¹, respectively (FIG. 12A). These results were consistent with the expected mechanism of action in which all four compounds form a covalent bond with the catalytic C145. In the thermal shift-binding assay, all four compounds stabilized the SARS-CoV-2 M^{pro} upon binding as reflected by the Tₘ shift to higher temperatures (FIG. 12B). As the tribromoacetamide is sterically hindered, the mechanism of action of Jun9-88-6R might involve the nucleophilic attack of the carbonyl by the C145 thiol to give a thiohemiketal intermediate, followed by a 1,2-shift of the sulfur to displace one bromide.

To provide additional evidence to support the proposed mechanism of action of covalent binding, we performed three additional experiments. First, to demonstrate the reversibility of the binding of Jun9-62-2R to M^{pro}, we incubated 10 μM of SARS-CoV-2 M^{pro} with 10 μM of Jun9-62-2R for 2 h and monitored the enzymatic activity of M^{pro} following 100-fold dilution of the mixture. It was found that no enzymatic activity was recovered (FIG. 12C). In contrast, the mixture with our previously developed non-covalent inhibitor 23R showed nearly complete recovery of enzymatic activity after dilution (FIG. 12C). These results suggest that the binding of Jun9-62-2R is irreversible while the binding of 23R is reversible. Second, we repeated the FRET assay of Jun9-62-2R with different pre-incubation times and found that longer pre-incubation time gave lower IC₅₀ values (FIG. 12D). This data is consistent with the mode of action of covalent inhibitors (see, Thorarensen, A.; et al., Bioorg. Med. Chem. 2021, 29, 115865). In contrast, pre-incubation of M^{pro} with the non-covalent inhibitor 23R did not lead to significant changes of the IC₅₀ value. Third, we used native mass spectrometry to detect the covalent adducts of M^{pro} with Jun9-62-2R, Jun9-89-2R, Jun9-88-6R, and Jun9-89-4R. The expected mass shifts of 482 Da and 526 Da were observed for Jun9-62-2R and Jun9-89-2R, respectively (FIGS. 12E and F). Interesting, the expected dibromoacetamide conjugate was not observed for Jun9-88-6R, suggesting this conjugate might not be stable. Instead, the mass shift corresponding to the monobromo thiol adduct was observed (FIG. 12G). For Jun9-89-4R, the mass shifts for both the chlorobromo and bromo thiol adducts were observed (FIG. 12H).

To further profile the cellular M^{pro} inhibition, we tested these four compounds in our recently developed FlipGFP assay (see, Xia, Z.; et al., ACS Pharmcol. Transl. Sci. 2021, 4 (4), 1408-1421; Drayman, N.; et al., Science 2021, 373 (6557), 931-936). Briefly, the GFP is split into two parts, the β1-9 template and the β10-11 strands. The β10 and β11 strands were engineered with K5-E5 linker such that they are restrained in the parallel form. Upon cleavage of the linker by M^{pro}, β10 and β11 become antiparallel and can associate with the β1-9 template, leading to the restoration of the GFP signal. In the FlipGFP assay, GFP signal is proportional to the M^{pro} enzymatic activity. It was found that all four compounds led to dose-dependent inhibition of the GFP signal with EC₅₀ values of 0.96 μM (Jun9-62-2R), 0.91 μM (Jun9-90-3R), 1.57 μM (Jun9-90-4R), and 0.92 μM (Jun9-88-6R). The EC₅₀ value for the positive control GC-376 was 1.80 μM (FIGS. 12I and J). This result indicate that these four compounds can potently inhibit the M^{pro} in the cellular content.

### Antiviral Activity of SARS-CoV-2 M^{pro} Inhibitors with Novel Reactive Warheads

The antiviral activity of the four lead compounds was evaluated in both Vero E6 cells and Caco2-hACE2 cells to exclude cell type dependent effect. Caco2-hACE2 with endogenous TMPRSS2 expression is a validated cell line for SARS-CoV-2 antiviral assay (see, Hoffmann, M.; et al., Cell 2020, 181 (2), 271-280 e8; Bertram, S.; et al., J. Virol. 2010, 84 (19), 10016-25; Stanifer, M. L.; et al., Cell Rep. 2020, 32 (1), 107863). Jun9-62-2R, Jun9-90-3R, Jun9-90-4R, and Jun9-88-6R inhibited SARS-CoV-2 in Vero E6 cells with EC₅₀ values of 0.90, 2.07, 1.10, and 0.58 μM, respectively. All four compounds maintained potent antiviral activity in Caco2-hACE2 cells with EC₅₀ values of 2.05, 3.24, 1.43, and 2.15 μM, respectively. In comparison, GC-376 inhibited SARS-CoV-2 in Vero E6 and Caco2-hACE2 cells with EC₅₀ values of 1.51 and 2.90 μM. The antiviral activity of Jun9-90-3R was further confirmed in Calu-3 cells with an EC₅₀ value of 2.00 μM.

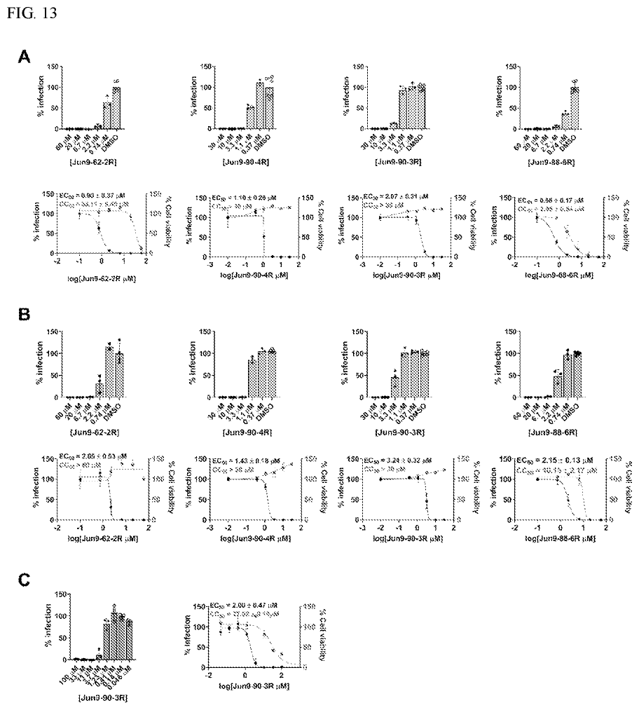

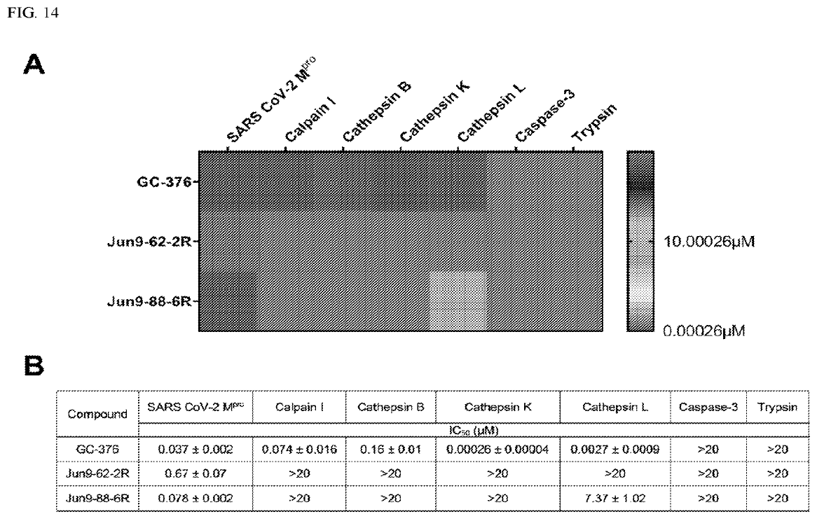

FIG. 13 shows antiviral activity of Jun9-62-2R, Jun9-90-3R, Jun9-90-4R, and Jun9-88-6R against SARS-CoV-2 in different cell lines. (A) Antiviral activity against SARS-CoV-2 in Vero E6 cells. (B) Antiviral activity against SARS-CoV-2 in Caco2-hACE2 cells. (C) Antiviral activity of Jun9-90-3R in Calu-3 cells. The results are average±standard deviation of three repeats.

### Profiling the Target Selectivity Against Host Proteases

Lack of target specificity is one of the major reasons that many cysteine protease inhibitors failed in the clinical trials. To profile the target specificity of these SARS-CoV-2 M^{pro} inhibitors with a novel reactive warhead, we selected Jun9-62-2R and Jun9-88-6R as representative examples and included the canonical GC-376 with an aldehyde reactive warhead for comparison. It was found that GC-376 had potent inhibition of the host proteases including calpain I, cathepsin B, cathepsin K, and cathepsin L with IC₅₀ values in the submicromolar and nanomolar range. GC-376 did not inhibit caspase-3 and trypsin (IC₅₀>20 μM) (FIG. 14). In comparison, both Jun9-62-2R and Jun9-88-6R had a significantly improved target selectivity and did not show potent inhibition against the host proteases calpain 1, cathepsin B, cathepsin K, cathepsin L, caspase-3, and trypsin. Jun9-88-6R had weak inhibition against cathepsin L with an IC₅₀ of 7.37 μM, conferring a 94-fold higher selectivity for inhibiting the SARS-CoV-2 M^{pro}. Collectively, the covalent SARS-CoV-2 M^{pro} inhibitors Jun9-62-2R with the dichloroacetamide warhead and Jun9-88-6R with the tribromoacetamide warhead have high target specificity against M^{pro} over host proteases.

### X-Ray Crystal Structures of SARS-CoV-2 M^{pro} in Complex with Jun9-62-2R and Jun9-57-3R

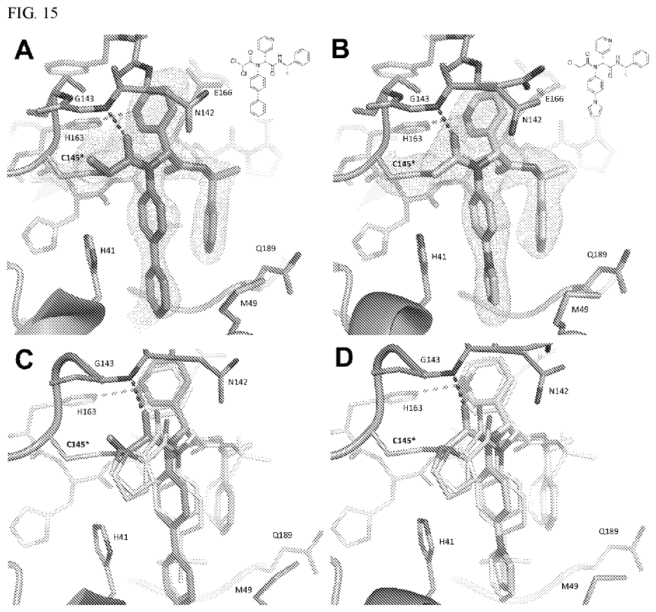

Using X-ray crystallography we solved the complex structures of SARS-CoV-2 M^{pro} with Jun9-57-3R (2.25 Å, PDB ID 7RN0) and Jun9-62-2R (2.30 Å, PDB ID 7RN1) (FIG. 15). Jun9-57-3R and Jun9-62-2R have nearly identical chemical features to their non-covalent progenitor 23R (Jun8-76-3A) (PDB ID 7KX5). As such, the binding poses are very similar. The pyridyl ring binds to the S1 pocket of M^{pro}, where it forms a hydrogen bond with His163. This hydrogen bond is critical for coordinating the Gln sidechain of its substrate, a residue it is uniquely selective for. Consequently, a hydrogen bond acceptor at this position confers tremendous potency to M^{pro} inhibitors. The phenylpyrrole (Jun9-57-3R) or biphenyl (Jun9-62-2R) moieties insert into the hydrophobic S2 pocket where they form nonpolar contacts and stack with the catalytic base, His41. An amide group linking the pyridyl ring to an a-methylbenzene group accepts a hydrogen bond from the mainchain of Glu166. This α-methylbenzene group flips down towards the core of the substrate channel, where it forms additional pi-stacking interactions with the biphenyl or phenylpyrrole moieties. The key distinction between Jun9-62-2R, Jun9-57-3R, and analogues Jun8-76-3A and ML188 is the presence of an electrophilic chloroacetamide warhead, which forms a covalent adduct with the catalytic cysteine Cys145 (FIG. 8C-D). The short distance of this covalent bond (1.8 Å) allows the inhibitor to press further into the oxyanion hole, causing the P2 benzene to rotate inwards by ˜40°. Likewise, the chloracetamide warhead is forced towards the catalytic core, causing the P1′ chloride of Jun9-57-3R to lie closer to Cys145 (2.8 Å) than the corresponding furyl oxygen of Jun8-76-3A (3.2Å).

### Conclusion

The majority of the reported M^{pro} inhibitors contain the aldehyde reactive warhead, which is known to have non-specific reactivity towards host proteins (see, Ma, X. R.; et al., ChemMedChem 2021, doi: 10.1002/cmdc.202100456; Steuten, K.; et al, ACS Infect. Dis. 2021, 7 (6), 1457-1468; Xia, Z.; et al., ACS Pharmcol. Transl. Sci. 2021, 4 (4), 1408-1421; Vandyck, K.; et al., Biochem. Biophys. Res. Commun. 2021, 555, 134-139). It should be noted that both the Pfizer M^{pro} inhibitors that are currently in clinical trials do not contain the aldehyde warhead (see, Owen, D. R.; et al., medRxiv 2021, 2021.07.28.21261232; Boras, B.; et al., bioRxiv 2020, 2020.09.12.293498). As such, we are interested in developing SARS-CoV-2 M^{pro} inhibitors with high target specificity. A highly specific M^{pro} inhibitor is also needed for target validation as it separates the effect of M^{pro} inhibition from host protease inhibition such as cathepsin L. It is known that host cathepsin L is important in SARS-CoV-2 replication in TMPRSS2-negative cell lines such as Vero E6, but not in TMPRSS2-positive cell lines such as Calu-3 (see, Shang, J.; et al., Proc. Natl. Acad. Sci. U S. A. 2020, 117 (21), 11727-11734). Experiments conducted during the course of developing embodiments for the present invention pertain to the discovery of dichloroacetamide, dibromoacetamide, 2-bromo-2, 2-dichloroacetamide, 2-chloro-2, 2-dibromoacetamide, and tribromoacetamide as novel cysteine reactive warheads. To the best of our knowledge, these warheads have not been explored in cysteine protease inhibitors. The promising lead compounds Jun9-62-2R with the dichloroacetamide warhead and Jun9-88-6R with the tribromoacetamide inhibited SARS-CoV-2 M^{pro} with IC₅₀ values of 0.43 μM and 0.08 μM, respectively. These two compounds also showed potent inhibition against SARS-CoV-2 in both Vero E6 and Caco2-hACE2 cells with EC₅₀ values in the single-digit to submicromolar range. Significantly, both Jun9-62-2R and Jun9-88-6R had high target specificity towards M^{pro} and did not inhibit the host proteases including calpain I, cathepsin B, cathepsin K, cathepsin L, caspase-3, and trypsin. In comparison, GC-376 was not selective and inhibited calpain I, cathepsin B, cathepsin K, and cathepsin L with comparable potency as M^{pro}. Regarding the translational potential of the di- and trihaloacetamide-containing M^{pro} inhibitors, the widely used antibiotic chloramphenicol contains the dichloroacetamide, suggesting Jun9-62-2R might be tolerated in vivo.

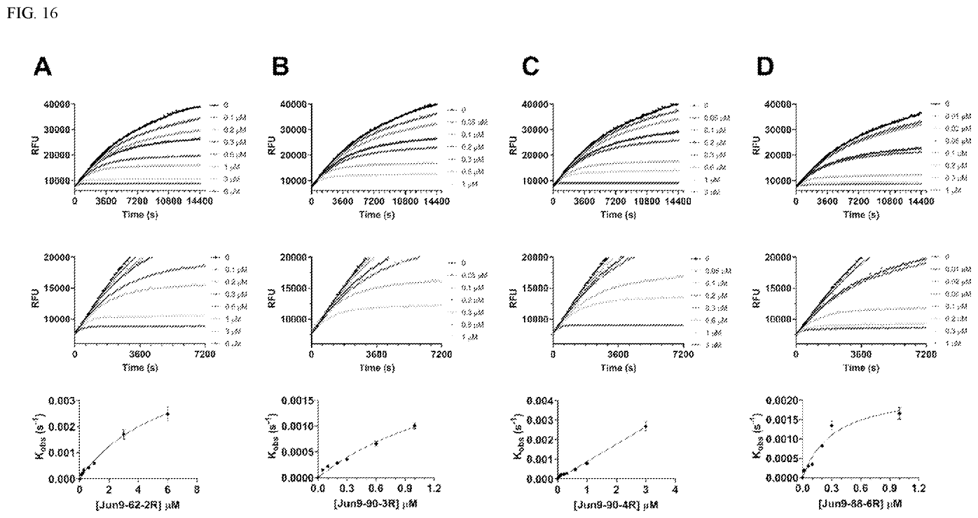

FIG. 16 shows enzymatic kinetic studies of Jun9-62-2R (A), Jun9-90-3R (B), Jun9-90-4R (C), and Jun9-88-6R (D) in inhibiting SARS-CoV-2 M^{pro}.

### Example VII

This example provides the materials and methods utilized in performing the experimetns described in Example VI.

### Protein Expression and Purification

SARS-CoV-2 main protease (M^{pro}) gene from strain BetaCoV/Wuhan/WIV04/2019 (GenBank: MN996528.1) was purchased from GenScript (Piscataway, NJ) with E. coli codon optimization and inserted into pET29a(+) plasmid. The M^{pro} genes were then subcloned into the pE-SUMO plasmid as previously described (see, Sacco, M. D.; et al., Sci. Adv. 2020, 6, eabe0751). The expression and purification procedures were previously described.¹ Cathepsin K (catalog no. 219461) and cathepsin L (catalog no. 219402) were purchased from EMD Millipore. Human cathepsin B (catalog no. CTB-H5222) was purchased from Acro Biosystems (Newark, DE), Calpain I (catalog no. C6108) and trypsin (catalog no. T6763) were purchased from Sigma-Aldrich, and caspas-3 (catalog no. 1083-25) was purchased from BioVision (Milpitas, CA)

### Differential Scanning Fluorimetry (DSF)

Direct binding of testing compounds to SARS CoV-2 M^{pro} was detected by differential scanning fluorimetry (DSF) using a Thermal Fisher QuantStudio 5 Real-Time PCR System as previously described (see, Ma, C.; et al., Cell Res. 2020, 30, 678-692). M^{pro} protein was diluted in enzymatic reaction buffer containing 20 mM HEPES, pH 6.5, 120 mM NaCl, 0.4 mM EDTA, 4 mM DTT, and 20% glycerol to a final concentration of 3 μM and incubated with 6 μM testing compounds at 30° C. for 30 min. DMSO was included as a reference. Fluorescence signal was recorded from 20 to 95° C. (incremental step of 0.05° C./s) after adding 1× SYPRO orange (Thermal Fisher, catalog no.: S6650). The melting temperature (Tₘ) was calculated as the mid log of the transition phase from the native to the denatured protein using a Boltzmann model in Protein Thermal Shift Software v1.3.

### Enzymatic Assays

IC₅₀ values for the testing compounds against SARS-CoV-2 M^{pro} was determined as previously described (see, Ma, C.; et al., Cell Res. 2020, 30, 678-692). Briefly, 100 nM M^{pro} was incubated with serial concentrations of the compounds at 30° C. for 30 min in 100 μl enzymatic reaction buffer (20 mM HEPES, pH 6.5, 120 mM NaCl, 0.4 mM EDTA, 4 mM DTT, and 20% glycerol). The proteolytic reactions were monitored in Cytation 5 imaging reader (Thermo Fisher Scientific) with filters for excitation at 360/40 nm and emission at 460/40 nm for 1 h after adding 1 ul of 1 mM of FRET substrate. The initial velocity of the proteolytic reaction was calculated by linear regression for the first 15 min of the kinetic progress curves. IC50 values were calculated in dose-response-variable slope (4 parameters) function in Prism 8.

Proteolytic reaction progress curve kinetics measurements with Jun9-66-2R, Jun9-90-3R, Jun9-90-4R and Jun9-88-6R were carried out as previously described with minor modification: 5 nM SARS-CoV-2 M^{pro} was added into 20 μM FRET-substrate premixed with serial concentrations of the compounds in 200 μl of reaction buffer at 30° C. to initiate the proteolytic reaction; the reaction was monitored for 4 h. The first two hours of kinetic curves were utilized in the curve fitting. The progression curves fittings were detailed described in previous publication (see, Ma, C.; et al., Cell Res. 2020, 30, 678-692). The k₂/K_{I} value is commonly used to evaluate the efficacy for covalent inhibitor. For compound Jun9-90-4R, we could not get accurate individual k₂ and K_{I} values, The calculated slope of K_{obs} over Jun9-90-4R concentration was deemed as k₂/K_{I} (see, Strelow, J. M., SLAS Discov. 2017, 22, 3-20).

Enzymatic reactions against host proteases (Calpain I, Cathepisn K, Cathespsin L, Caspase-3 and Trypisn) were carried out as previously described (see, Xia, Z.; et al., ACS Pharmacol. Transl. Sci. 2021). Cathepsin B assay reaction was carried out as follows: Cathepsin B (catalog no. CTB-H5222) was diluted into 100 nM final concentration in a buffer containing 20 mM sodium acetate pH5.5, 1 mM EDTA and 2 mM DTT and the mixture was incubated for 30 min at 30° C. Activated Cathepsin B was further diluted to 500 pm in the reaction buffer (100 mM MES pH6.0, 1 mM EDTA, 2 mM DTT and 0.01% TWEEN 20). Then 1 μl of serial concentrations of testing compounds were added and incubated for 30 min at 30° C., and the enzymatic reaction was initiated by adding 1 μl of 500 μM of FRET substrate Z-Phe-Arg-AMC (BACHEM, catalog #. 4003379.0050); the IC50 value was calculated same as cathepsin K and L.

### Cellular-Based FlipGFP M^{pro} Assay

Plasmid pcDNA3-SARS2-M^{pro}-flipGFP-T2A-mCherry was construct as previously described (see, Xia, Z.; et al., ACS Pharmacol. Transl. Sci. 2021). SARS-CoV-2 M^{pro} expression plasmid pcDNA3.1 SARS-CoV-2 Mpro were ordered from Genscript (Piscataway NJ) with codon optimization. The FlipGFP M^{pro} assay was carried out exact as previously described (see, Xia, Z.; et al., ACS Pharmacol. Transl. Sci. 2021). Briefly, 50 ng of pcDNA3-SARS2-M^{pro}-flipGFP-T2A-mCherry plasmid and 50 ng of protease expression plasmid pcDNA3.1 SARSCoV-2 M^{pro} were transfected into 293T cells with transfection reagent TranslT-293 (Mirus catalog no. MIR 2700) according to the manufacturer's protocol. Three hours after transfection, testing compound was added to each well at 100-fold dilution. Two days after transfection, GFP and mCherry signals were taken with Cytation 5 imaging reader (Biotek) using GFP and mCherry channels via 10× objective lens and were analyzed with Gen5 3.10 software (Biotek). SARS-CoV-2 M^{pro} protease activity was evaluated as the ratio of GFP signal over mCherry signal, the mCherry signal alone in the presence of testing compounds was used to evaluate the compound cytotoxicity.

### X-Ray Crystallization

Jun9-57-3R and Jun9-62-2R were added to 15 mg/mL SARS-CoV-2 M^{pro} to a final concentration of 2 mM and incubated overnight at 4° C. The protein-inhibitor slurry was spun down at 13,000 g for 1 minute. The supernatant was removed and diluted to 5 mg/mL with protein stock buffer (20 mM Tris pH 7.5, 200 mM NaCl, 1 mM DTT). Crystals were grown by mixing protein with an equal volume of crystallization buffer (25% PEG 3350, 0.2 M AmSO4, 0.1 M HEPES 7.5) in a vapor diffusion, hanging drop apparatus. Crystals typically grew to full size in several days, at which time they were transferred to a cryoprotectant solution of 30% PEG 3350, 0.2 M AmSO4, 0.1 M HEPES 7.5, and 15% glycerol for 5 seconds and flash-frozen in liquid nitrogen.

X-ray diffraction data for SARS-CoV-2 M^{pro} with Jun9-57-3R and Jun9-62-2R were collected on the SBC 19-ID and SER-CAT 22ID beamlines at the Advanced Photon Source (APS) in Argonne, IL. Diffraction data was indexed and processed with the CCP4 versions of iMosflm (see, Otwinowski, Z.; Minor, W., Methods Enzymol. 1997, 276, 307-26). Diffraction data was then scaled with AIMLESS and molecular replacement performed with MOLREP (see, Vagin, A.; Teplyakov, A., Acta Crystallogr. Sect. D Biol. Crystallogr. 2010, 66, 22-25). Structural refinement was performed with REFMACS and COOT (see, Murshudov, G. N.; et al., Acta Crystallogr. Sect. D Biol. Crystallogr. 2011, 67, 355-367; Emsley, P.; Cowtan, K., Acta Crystallogr. Sect. D Biol. Crystallogr. 2004, 60, 2126-2132).

SARS-CoV-2 M^{pro} complex structures are deposited in the protein data bank under the accession numbers 7RN0 (Jun9-57-3R) and 7RN1 (Jun9-62-2R). Crystallographic statistics are presented in Table 5.

TABLE 5

| Data Collection | PDB ID 7RN0 | PDB ID 7RN1 |
|---|---|---|
| Inhibitor | Jun9-57-3R | Jun9-62-2R |
| Space Group | I 1 2 1 | C 1 2 1 |
| Cell Dimension | | |
| a, b, c (Å) | 45.42, 53.43, 112.03 | 114.27, 53.26, 45.46 |
| α , β, γ (°) | 90.00, 101.37, 90.00 | 90, 102.82, 90 |
| Resolution (Å) | 48.05-2.25 | 48.05-2.30 |
| | (2.33-2.25) | (2.38-2.30) |
| R_{merge} | 0.071 (0.709) | 0.088 (0.423) |
| <\|>/σ<\|> | 7.6 (2.2) | 7.2 (2.4) |
| Completeness (%) | 92.4 (95.4) | 76.7 (82.2) |
| Multiplicity | 2.7 (2.8) | 2.9 (2.9) |
| Refinement | | |
| Resolution (Å) | 38.70-2.25 | 48.05-2.30 |
| | (2.33-2.25) | (2.38-2.30) |
| No. reflections/free | 11607/597 | 9032/434 |
| R_{work}/R_{free} | 0.219/0.248 | 0.204/0.230 |
| No. Atoms | | |
| Protein | 2335 | 2361 |
| Ligand/Ion | 45 | 52 |
| Water | 22 | 7 |
| B-Factors (Å²) | | |
| Overall | 58.26 | 44.70 |
| Protein | 58.52 | 44.83 |
| Ligand/Ion | 49.15 | 39.72 |
| Solvent | 49.34 | 37.61 |
| RMS Deviations | | |
| Bond Lengths (Å) | 0.015 | 0.014 |
| Bond Angles (°) | 1.92 | 1.91 |
| Ramachandran Favored (%) | 95.00 | 96.04 |
| Ramachandran Allowed (%) | 5.00 | 3.96 |
| Ramachandran Outliers (%) | 0.00 | 0.00 |
| Rotameric Outliers (%) | 2.32 | 1.91 |
| Clashscore | 8.76 | 5.71 |

(* Values in parentheses refer to the last resolution shell)

### Chemistry

Chemicals were ordered from commercial sources and were used without further purification. All final compounds were purified by flash column chromatography. ¹H and ¹³C NMR spectra were recorded on a Bruker-400 NMR spectrometer. Chemical shifts are reported in parts per million referenced with respect to residual solvent DMSO-d6) 2.50 ppm and from internal standard tetramethylsilane (TMS) 0.00 ppm. The following abbreviations were used in reporting spectra: s, singlet; d, doublet; t, triplet; q, quartet; m, multiplet; dd, doublet of doublets. All reactions were carried out under N2 atmosphere unless otherwise stated. HPLC-grade solvents were used for all reactions. Flash column chromatography was performed using silica gel (230-400 mesh, Merck). Low-resolution mass spectra were obtained using an ESI technique on a 3200 Q Trap LC/MS/MS system (Applied Biosystems). The purity was assessed by using Shimadzu LC-MS with Waters XTerra MS C-18 column (part #186000538), 50×2.1 mm, at a flow rate of 0.3 mL/min; λ=250 and 220 nm; mobile phase A, 0.1% formic acid in H₂O, and mobile phase B′, 0.1% formic in 60% isopropanol, 30% CH₃CN and 9.9% H₂O. The diastereomers were separated using the ACCQPrep HP150 HPLC system equipped with the Chiral HPLC Lux®5 μM i-Amylose-3 chiral HPLC column.

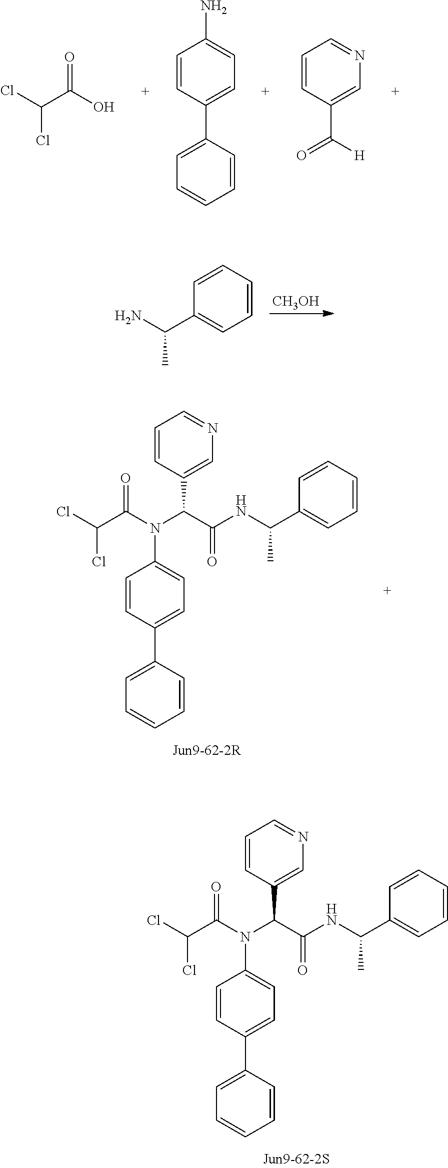

### General Synthesis Procedure

The M^{pro} inhibitors were synthesized using the Ugi four-component reaction methodology as described previously (see, Kitamura, N.; et al., J. Med. Chem. 2021). Briefly, aldehyde (1 mmol) and amine (1 mmol) were mixed in methanol (10 ml) and stirred at room temperature for 30 minutes. Then acid (1 mmol) and isocyanide (1 mmol) were added sequentially. The resulting mixture was stirred at room temperature overnight. Solvent was removed under reduced pressure and the residue was purified by silica gel flash column chromatography (CH₂Cl₂ to CH₂Cl₂/MeOH=10:1) to afford the target product.

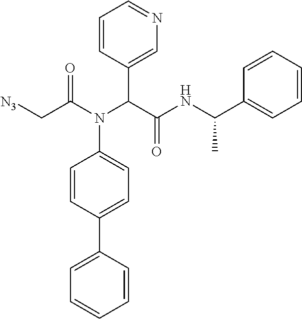 [SMILES: C[C@H](NC(=O)C(c1cccnc1)N(C(=O)CN=[N+]=[N-])c1ccc(-c2ccccc2)cc1)c1ccccc1]

N-([1,1′-biphenyl]-4-yl)-2-azido-N-(2-oxo-2-(((S)-1-phenylethyl)amino)-1-(pyridin-3-yl)ethyl)acetamide (Jun9-61-1). White solid, 50% yield, dr=1:1. ¹H NMR (500 MHz, DMSO-d6) δ8.78-8.72 (m, 1H), 8.50-8.19 (m, 2H), 7.64-7.03 (m, 16H), 6.25 (s, 0.5H), 6.22 (s, 0.5H), 5.14-4.85 (m, 1H), 3.84-3.75 (m, 1H), 3.68-3.61 (m, 1H), 1.40 (d, J=7.0 Hz, 1.5H), 1.28 (d, J=7.0 Hz, 1.5H). ¹³C NMR (125 MHz, DMSO-d6) δ168.0, 167.8, 167.4, 167.3, 151.2, 151.1, 148.9, 148.8, 144.2, 143.9, 139.7, 138.6, 137.4, 137.3, 136.8, 131.2, 130.6, 130.2, 128.9, 128.9, 128.2, 128.1, 127.8, 126.8, 126.7, 126.6, 126.6, 126.2, 125.8, 123.0, 122.7, 61.7, 50.6, 48.4, 48.3, 22.3, 22.1. C₂₉H₂₆N₆O₂, HRMS calculated for m/z [M+H]⁺: 491.219549 (calculated), 491.21900 (found).

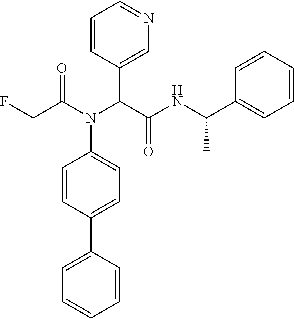 [SMILES: C[C@H](NC(=O)C(c1cccnc1)N(C(=O)CF)c1ccc(-c2ccccc2)cc1)c1ccccc1]

N-([1,1′-biphenyl]-4-yl)-2-fluoro-N-(2-oxo-2-(((S)-1-phenylethyl)amino)-1-(pyridin-3-yl)ethyl)acetamide (Jun9-61-4). White solid, 79% yield, dr=1:1. ¹H NMR (500 MHz, DMSO-d6) δ8.90-8.61 (m, 1H), 8.47-8.22 (m, 2H), 7.66-7.08 (m, 16H), 6.25 (s, 0.5H), 6.23 (s, 0.5H), 5.04-4.93 (m, 1H), 4.80-4.51 (m, 2H), 1.40 (d, J=7.0 Hz, 1.5H), 1.28 (d, J=7.0 Hz, 1.5H). ¹³C NMR (125 MHz, DMSO-d6) δ167.9, 167.7, 166.5, 166.5, 166.4, 166.3, 151.2, 151.1, 148.9, 148.9, 144.2, 143.9, 139.8, 138.6, 137.4, 137.4, 135.9, 135.8, 131.2, 130.5, 130.0, 129.0, 128.9, 128.2, 128.1, 127.9, 126.8, 126.7, 126.7, 126.6, 126.2, 125.8, 123.0, 122.8, 79.3, 78.0, 61.3, 61.2, 48.4, 48.3, 22.3, 22.1. C₂₉H₂₆FN₃O₂, HRMS calculated for m/z [M+H]⁺: 468.208730 (calculated), 468.20818 (found).

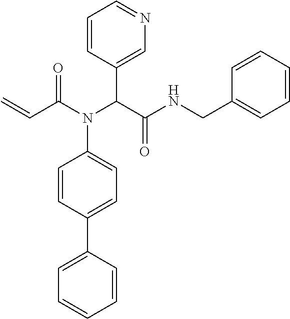 [SMILES: C=CC(=O)N(c1ccc(-c2ccccc2)cc1)C(C(=O)NCc1ccccc1)c1cccnc1]

N-([1,1′-biphenyl]-4-yl)-N-(2-(benzylamino)-2-oxo-1-(pyridin-3-yl)ethyl)acrylamide (Jun10-15-2). White solid, 75% yield. ¹H NMR (500 MHz, DMSO-d6) δ8.78 (t, J=6.0 Hz, 1H), 8.40-8.28 (m, 2H), 7.66-7.12 (m, 16H), 6.33-6.20 (m, 2H), 6.00-5.93 (m, 1H), 5.61 (dd, J=10.5, 2.0 Hz, 1H), 4.47-4.33 (m, 2H). ¹³C NMR (125 MHz, DMSO-d6) δ168.9, 164.7, 151.1, 148.8, 139.3, 139.1, 138.7, 138.2, 137.5, 131.3, 130.9, 128.9, 128.2, 127.9, 127.7, 127.2, 126.7, 126.5, 122.9, 61.8, 42.4. C₂₉H₂₅N₃O₂, HRMS calculated for mlz [M+H]⁺: 448.202502, (calculated), 448.20195 (found).

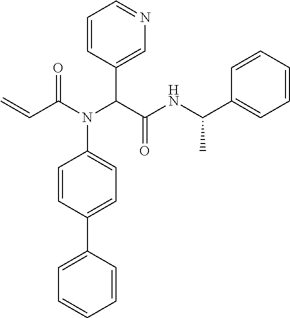 [SMILES: C=CC(=O)N(c1ccc(-c2ccccc2)cc1)C(C(=O)N[C@@H](C)c1ccccc1)c1cccnc1]

N-([1,1′-biphenyl]-4-yl)-N-(2-oxo-2-(((S)-1-phenylethyl)amino)-1-(pyridin-3-yl)ethyl)acrylamide (Jun9-51-3), dr=1.4:1. White solid, 84% yield. ¹H NMR (500 MHz, DMSO-d6) δ8.76-0.70 (m, 1H), 8.41-8.24 (m, 2H), 7.62-7.07 (m, 15H), 6.33-6.17 (m, 2H), 5.98-5.88 (m, 1H), 5.62-5.55 (m, 1H), 5.07-4.96 (m, 1H), 1.38 (d, J=7.0 Hz, 1.75H), 1.27 (d, J=7.0 Hz, 1.25H). ¹³C NMR (125 MHz, DMSO-d6) 168.2, 168.0, 164.6, 164.6, 151.2, 151.1, 148.8, 144.3, 140.0, 139.3, 139.3, 138.7, 138.1, 138.1, 137.4, 137.4, 131.4, 131.1, 130.7, 128.9, 128.9, 128.2, 128.1, 128.0, 127.7, 126.7, 126.6, 126.5, 126.2, 125.8, 123.0, 122.7, 61.5, 48.4, 48.3, 22.3, 22.2. C₃₀H₂₇N₃O₂, HRMS calculated for m/z [M+H]⁺: 462.218152 (calculated), 462.21760 (found).

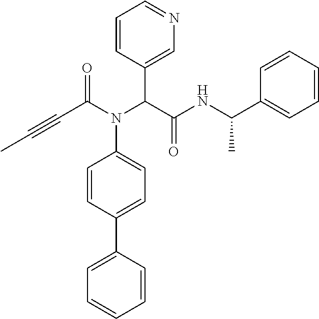 [SMILES: CC#CC(=O)N(c1ccc(-c2ccccc2)cc1)C(C(=O)N[C@@H](C)c1ccccc1)c1cccnc1]

N-([1,1′-hiphenyl]-4-yl)-N-(2-oxo-2-(((S)-1-phenylethyl)amino)-1-(pyridin-3-yl)ethyl)but-2-ynamide (Jun9-62-1), dr=1:1. White solid, 50% yield. ¹H NMR (500 MHz, DMSO-d6) δ8.74 (d, J=7.5 Hz, 1H), 8.42-8.25 (m, 2H), 7.56-7.09 (m, 16H), 6.18 (d, J=13.0 Hz, 1H), 5.05-4.95 (m, 1H), 1.69 (d, J=6.5 Hz, 3H), 1.37 (d, J=7.0 Hz, 1.5H), 1.27 (d, J=7.0 Hz, 1.5H). ¹³C NMR (125 MHz, DMSO-d6) 167.7, 167.5, 153.5, 153.5, 151.1, 151.0, 148.9, 148.9, 144.2, 143.9, 139.1, 139.1, 138.7, 138.2, 138.2, 137.4, 137.3, 131.4, 130.5, 130.2, 128.9, 128.2, 128.1, 127.7, 126.7, 126.7, 126.5, 126.1, 126.1, 125.8, 123.1, 122.8, 90.8, 90.7, 74.3, 74.3, 61.3, 61.3, 48.5, 48.3, 22.3, 22.1, 3.2, 3.2. C₃₁H₂₇N₃O₂, HRMS calculated for m/z [M+H]⁺: 474.218152 (calculated), 474.21760 (found).

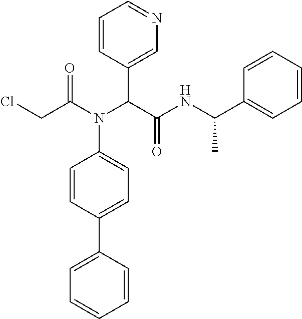 [SMILES: C[C@H](NC(=O)C(c1cccnc1)N(C(=O)CCl)c1ccc(-c2ccccc2)cc1)c1ccccc1]

2-(N-{[1,1′-biphenyl]-4-yl}-2-chloroacetamido)-N-[(1S)-1-phenylethyl]-2-(pyridin-3-yl)acetamide (Jun9-54-1). dr=1:1. White solid, 88% yield. ¹H NMR (400 MHz, DMSO-d₆) δ8.73 (dd, J=10.4, 7.8 Hz, 1H), 8.52-8.20 (m, 2H), 7.69-7.29 (m, 10H), 7.29-7.03 (m, 5H), 6.20 (d, J=6.8 Hz, 1H), 5.00 (h, J=7.3 Hz, 1H), 4.19-3.90 (m, 2H), 1.39 (d, J=7.0 Hz, 1.5H), 1.26 (d, J=7.0 Hz, 1.5H). ¹³C NMR (101 MHz, DMSO-d₆) δ167.74, 167.59, 165.47, 165.41, 151.05, 150.97, 148.81, 144.15, 143.80, 139.63, 138.52, 137.31, 137.21, 137.15, 131.20, 130.57, 130.18, 128.86, 128.85, 128.21, 128.15, 128.01, 127.75, 126.70, 126.63, 126.55, 126.49, 126.48, 126.10, 125.68, 122.96, 122.67, 61.84, 48.32, 48.20, 42.99, 22.20, 22.01. C₂₉H₂₆ClN₃O₂, HRMS calculated for m/z [M+H]⁺: 484.179180 (calculated), 484.17863 (found).

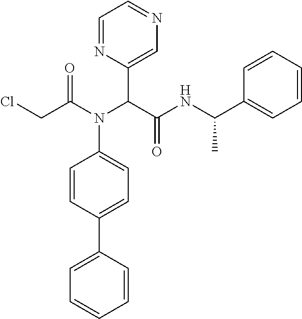 [SMILES: C[C@H](NC(=O)C(c1cnccn1)N(C(=O)CCl)c1ccc(-c2ccccc2)cc1)c1ccccc1]

2-(N-{[1,1′-biphenyl]-4-yl}-2-chioroacetamido)-N-[(1S)-1-phenylethyl]-2-(pyrazin-2-yl)acetamide (Jun9-59-1). White solid, 82% yield, dr=1:1. ¹H NMR (500 MHz, DMSO-d₆) δ8.76 (dd, J=28.6, 7.8 Hz, 1H), 8.62-8.33 (m, 3H), 7.73-7.12 (m, 14H), 6.33 (d, J=3.9 Hz, 1H), 4.99 (dp, J=21.6, 7.1 Hz, 1H), 4.24-3.91 (m, 2H), 1.34 (d, J=7.1 Hz, 2H), 1.30 (d, J=7.0 Hz, 1H). ¹³C NMR (126 MHz, DMSO-d₆) δ166.16, 166.03, 165.74, 160.02, 150.97, 150.82, 145.92, 145.81, 144.05, 143.90, 143.74, 143.68, 143.47, 139.87, 139.82, 138.66, 130.79, 130.71, 128.94, 128.28, 128.21, 128.11, 127.84, 126.88, 126.85, 126.73, 126.68, 126.63, 126.60, 126.13, 125.95, 125.93, 64.41, 64.34, 48.43, 48.37, 42.89, 22.17, 22.14. C₂₈H₂₅ClN₄O₂, HRMS calculated for m/z [M+H]⁺: 485.174429 (calculated), 485.17388 (found).

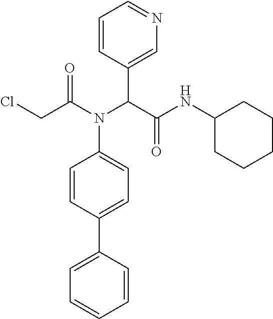 [SMILES: O=C(NC1CCCCC1)C(c1cccnc1)N(C(=O)CCl)c1ccc(-c2ccccc2)cc1]

N-([1,1′-biphenyl]-4-yl)-2-chloro-N-(2-(cyclohexylamino)-2-oxo-1-(pyridin-3-yl)ethyl)acetamide (Jun9-55-2). White solid, 85% yield. ¹H NMR (500 MHz, DMSO-d6) δ8.45-8.40 (m, 1H), 7.55-7.30 (m, 8H), 7.08 (dd, J=8.0, 5.0 Hz, 1H), 6.65 (d, J=7.5 Hz, 1H), 6.12 (s, 1H), 3.98-3.87 (m, 2H), 3.84-3.72 (m, 1H), 2.06-1.56 (m, 5H), 1.44-0.98 (m, 5H). ¹³C NMR (125 MHz, DMSO-d6) δ167.7, 167.3, 151.2, 149.6, 142.1, 139.4, 138.1, 136.8, 130.9, 130.4, 128.9, 128.0, 127.1, 123.4, 62.6, 49.1, 42.5, 32.7, 32.7, 25.5, 24.9, 24.8. C₂₇H₂₈ClN₃O₂, HRMS calculated for m/z [M+H]⁺: 462.194830 (calculated), 462.19428 (found).

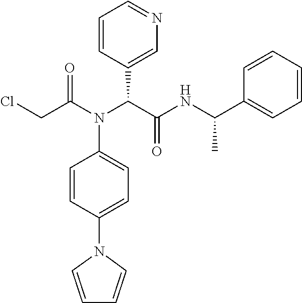 [SMILES: C[C@H](NC(=O)[C@@H](c1cccnc1)N(C(=O)CCl)c1ccc(-n2cccc2)cc1)c1ccccc1]

N-(4-(1H-pyrrol-1-yl)phenyl)-2-chloro-N-((R)-2-oxo-2-(((S)-1-phenylethyl)amino)-1-(pyridin-3-yl)ethyl)acetamide (Jun9-57-3R). White solid, 30% yield. ¹H NMR (500 MHz, DMSO-d6) δ8.64 (d, J=8.0 Hz, 1H), 8.34-8.22 (m, 2H), 7.51-7.02 (m, 13H), 6.18-6.10 (m, 3H), 5.06-4.85 (m, 1H), 4.00-3.85 (m, 2H), 1.18 (d, J=7.0 Hz, 3H). ¹³C NMR (125 MHz, DMSO-d6) δ167.9, 165.6, 151.1, 149.0, 143.9, 139.2, 137.3, 134.5, 132.1, 130.6, 128.2, 126.7, 126.2, 123.1, 118.7, 118.5, 110.9, 61.8, 48.3, 43.1, 22.1. C₂₇H₂₅ClN₄O₂, HRMS calculated for mlz [M+H]⁺: 473.174429 (calculated), 473.17388 (found).

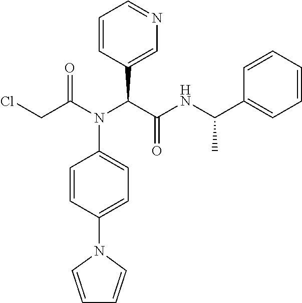 [SMILES: C[C@H](NC(=O)[C@H](c1cccnc1)N(C(=O)CCl)c1ccc(-n2cccc2)cc1)c1ccccc1]

N-(4-(1H-pyrrol-1-yl)phenyl)-2-chloro-N-((S)-2-oxo-2-4(S)-1-phenylethyl)amino)-1-(pyridin-3-yl)ethyl)acetamide (Jun9-57-3S). White solid, 31% yield. ¹HNMR (500 MHz, DMSO-d6) δ8.67 (d, J=7.5 Hz, 1H), 8.24 (dd, J=4.5, 1.5 Hz, 1H), 8.17 (d, J=2.0 Hz, 1H), 7.52-6.91 (m, 13H), 6.24-6.04 (m, 3H), 5.05-4.81 (m, 1H), 3.95 (dd, J=39.0, 14.0 Hz, 2H), 1.31 (d, J=7.0 Hz, 3H). ¹³C NMR (125 MHz, DMSO-d6) δ167.7, 165.7, 151.2, 149.0, 144.2, 139.2, 137.4, 134.5, 132.1, 130.2, 128.1, 126.6, 125.7, 122.8, 118.7, 118.6, 110.9, 61.8, 48.4, 43.1, 22.3. C₂₇H₂₅ClN₄O₂, HRMS calculated for m/z [M+H]⁺: 473.174429 (calculated), 473.17388 (found).

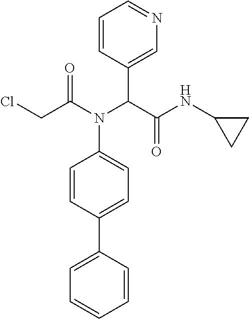 [SMILES: O=C(NC1CC1)C(c1cccnc1)N(C(=O)CCl)c1ccc(-c2ccccc2)cc1]

N-([1,1′-biphenyl]-4-yl)-2-chloro-N-(2-(cyclopropylamino)-2-oxo-1-(pyridin-3-yl)ethyl)acetamide (Jun9-57-2). White solid, 75% yield. ¹H NMR (500 MHz, DMSO-d6) δ8.36 (d, J=4.0 Hz, 1H), 8.32 (d, J=2.5 Hz, 2H), 7.63-7.32 (m, 11H), 7.15 (dd, J=8.0, 4.5 Hz, 1H), 6.02 (s, 1H), 4.03 (dd, J=35.5, 14.0 Hz, 2H), 2.74-2.61 (m, 1H), 0.70-0.55 (m, 2H), 0.43-0.26 (m, 2H). ¹³C NMR (125 MHz, DMSO-d6) δ170.1,166.0, 151.4, 149.4, 131.7, 131.0, 129.4, 128.3, 127.3, 127.1, 123.5, 62.4, 43.6, 23.0, 6.1. C₂₄H₂₂ClN₃O₂, HRMS calculated for m/z [M+H]⁺: 420.147880 (calculated), 420.14733 (found).

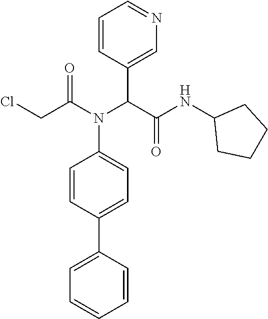 [SMILES: O=C(NC1CCCC1)C(c1cccnc1)N(C(=O)CCl)c1ccc(-c2ccccc2)cc1]

2-(N-{[1,1′-biphenyl]-4-yl}-2-chloroacetamido)-N-cyclopentyl-2-(pyridin-3-yl)acetamide (Jun9-55-1). White solid, 72% yield. ¹H NMR (500 MHz, DMSO-d₆) δ8.46-8.28 (m, 2H), 8.23 (d, J=7.0 Hz, 1H), 7.78-7.49 (m, 4H), 7.49-7.25 (m, 5H), 7.15 (dd, J=7.9, 4.7 Hz, 1H), 6.09 (s, 1H), 4.17-4.02 (m, 2H), 4.02-3.88 (m, 1H), 1.94-1.67 (m, 2H), 1.67-1.32 (m, 5H), 1.32-1.14 (m, 1H). ¹³C NMR (126 MHz, DMSO-d₆) δ168.58, 167.88, 165.47, 150.94, 148.79, 139.70, 138.62, 137.33, 137.29, 131.26, 130.78, 129.00, 128.94, 127.83, 126.77, 126.57, 122.99, 61.92, 50.71, 43.11, 32.13, 31.85, 23.48, 23.43. C₂₆H₂₆ClN₃O₂, HRMS calculated for m/z [M+H]⁺: 448.179180 (calculated), 448.17863 (found).

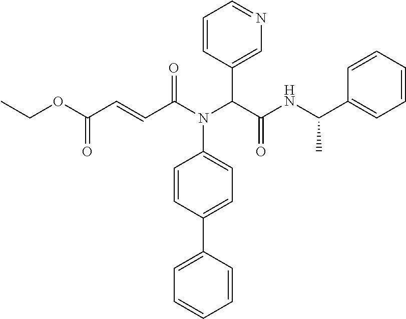 [SMILES: CCOC(=O)/C=C/C(=O)N(c1ccc(-c2ccccc2)cc1)C(C(=O)N[C@@H](C)c1ccccc1)c1cccnc1]

Ethyl (E)-4-([1,1′-biphenyl]-4-yl(2-oxo-2-(((S)-1-phenylethyl)amino)-1-(pyridin-3-yl)ethyl)amino)-4-oxobut-2-enoate (Jun9-72-3), dr=1:1. White solid, 69% yield. ¹H NMR (500 MHz, DMSO-d6) δ8.78 (t, J=7.6 Hz, 1H), 8.57-8.16 (m, 2H), 7.79-7.00 (m, 16H), 6.82-6.53 (m, 2H), 6.31 (d, J=11.2 Hz, 1H), 5.17-4.85 (m, 1H), 4.27-3.90 (m, 2H), 1.40 (d, J=7.2 Hz, 1.5H), 1.29 (d, J=7.2 Hz, 1.5H), 1.17-1.11 (m, 3H). ¹³C NMR (125 MHz, DMSO-d6) δ167.7, 167.6, 164.7, 164.7, 163.0, 163.0, 151.2, 151.1, 148.9, 144.2, 143.9, 139.6, 138.5, 137.4, 137.4, 134.3, 131.3, 130.7, 130.3, 130.3, 129.0, 128.9, 128.2, 128.1, 127.9, 126.8, 126.7, 126.6, 126.6, 126.2, 125.8, 123.1, 122.8, 61.9, 60.8, 60.8, 48.4, 48.4, 22.3, 22.1, 13.9. C₃₃H₃₁N₃O₄, HRMS calculated for m/z [M+H]⁺: 534.239282 (calculated), 534.23873 (found).

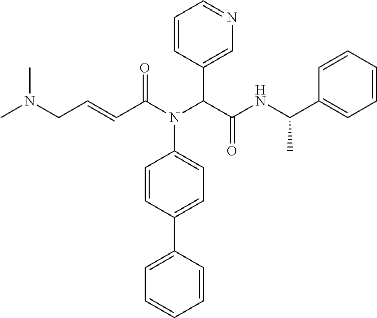 [SMILES: C[C@H](NC(=O)C(c1cccnc1)N(C(=O)/C=C/CN(C)C)c1ccc(-c2ccccc2)cc1)c1ccccc1]

(2E)-N-{[1,1′-biphenyl]-4-yl}-4-(dimethylamino)-N-({[(1S)-1phenylethyl]carbamoyl}-(pyridine-3-yl)methyl)but-2-enamide (Jun10-31-4). dr=1:1. White solid, 75% yield. ¹H NMR (500 MHz, DMSO-d₆) δ8.79-8.65 (m, 1H), 8.44-8.21 (m, 2H), 7.68-7.02 (m, 15H), 6.84-6.53 (m, 1H), 6.31 (s, 0.5H), 6.28 (s, 0.5H), 5.81-5.56 (m, 1H), 5.13-4.89 (m, 1H), 2.94-2.74 (m, 2H), 2.01 (s, 3H), 2.00 (s, 3H), 1.37 (d, J=7.0 Hz, 1.5H), 1.26 (d, J=7.0 Hz, 1.5H). ¹³C NMR (126 MHz, DMSO-d₆) δ168.23, 168.10, 164.66, 164.63, 151.14, 151.07, 148.73, 144.35, 143.96, 142.37, 139.03, 139.01, 138.63, 138.32, 138.25, 137.36, 137.33, 131.43, 131.41, 131.21, 130.80, 128.93, 128.91, 128.27, 128.19, 128.05, 127.73, 126.66, 126.57, 126.52, 126.48, 126.19, 125.92, 125.74, 123.08, 122.97, 122.68, 61.42, 59.62, 59.61, 48.35, 48.24, 44.85, 22.31, 22.17. C₃₃H₃₄N₄O₂, HRMS calculated for m/z [M+H]⁺: 519.276001 (calculated), 519.27545 (found).

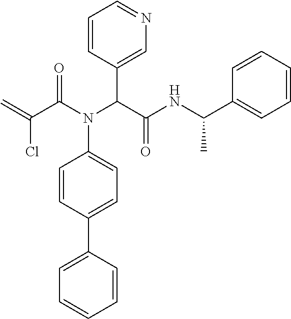 [SMILES: C=C(Cl)C(=O)N(c1ccc(-c2ccccc2)cc1)C(C(=O)N[C@@H](C)c1ccccc1)c1cccnc1]

N-{[1,1′-biphenyl]-4-yl}-2-chloro-N-({[(1S)-1-phenylethyl]carbamoyl}(pyridin-3-yl)methyl)prop-2-enamide (Jun10-38-2). dr=1:1. White solid, 72% yield. ¹H NMR (500 MHz, DMSO-d₆) δ8.82-8.67 (m, 1H), 8.47-8.23 (m, 2H), 7.75-7.54 (m, 2H), 7.54-7.14 (m, 12H), 7.12-7.00 (m, 1H), 6.22 (s, 0.5H), 6.19 (s, 0.5H), 5.64-5.43 (m, 2H), 5.13-4.86 (m, 1H), 1.36 (d, J=7.0 Hz, 1.5H), 1.27 (d, J=7.0 Hz, 1.5H). ¹³C NMR (126 MHz, DMSO-d₆) δ167.46, 167.33, 164.30, 164.25, 151.16, 151.11, 149.00, 144.20, 143.77, 139.12, 138.54, 138.09, 138.05, 137.42, 131.53, 131.49, 131.23, 131.19, 130.51, 130.13, 128.90, 128.89, 128.22, 128.09, 127.74, 126.72, 126.64, 126.47, 126.15, 125.76, 123.06, 122.76, 119.96, 119.94, 61.89, 61.86, 48.45, 48.26, 22.22, 22.12. C₃₀H₂₆ClN₃O₂, HRMS calculated for m/z [M+H]⁺: 496.179180 (calculated), 496.17863 (found).

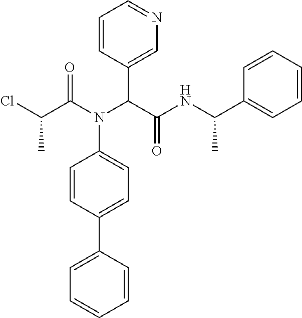 [SMILES: C[C@H](Cl)C(=O)N(c1ccc(-c2ccccc2)cc1)C(C(=O)N[C@@H](C)c1ccccc1)c1cccnc1]

(2S)-N-([1,1′-biphenyl]-4-yl)-2-chloro-N-(2-oxo-2-(((S)-1-phenylethyl)amino)-1-(pyridin-3-yl)ethyl)propanamide (Jun9-77-1), dr=1:1. White solid, 74% yield. ¹H NMR (500 MHz, DMSO-d6) δ8.70 (d, J=7.5 Hz, 1H), 8.51-8.39 (m, 2H), 7.62-7.45 (m, 5H), 7.39-7.33 (m, 3H), 7.31-7.26 (m, 1H), 7.17-7.07 (m, 3H). 7.05-6.99 (m, 2H), 6.16 (s, 1H), 4.9-4.83 (m, 1H), 4.25 (q, J=6,5 Hz, 1H), 1.43 (d, J=7.0 Hz, 3H), 1.27 (d, J=7.0 Hz, 3H). ¹³C NMR (125 MHz, DMSO-d6) δ168.7, 166.7,147.7, 145.9, 144.1, 141.3, 140.1, 138.6, 137.2, 132.2, 131.1, 129.0, 128.2, 127.9, 127.1, 126.7, 126.6, 125.8, 124.3, 61.8, 51,0, 48.6, 22.2, 21.2. C₃₀H₂₈ClN₃O₂, HRMS calculated for m/z [M+H]⁺: 498.194830 (calculated), 498.19428 (found).

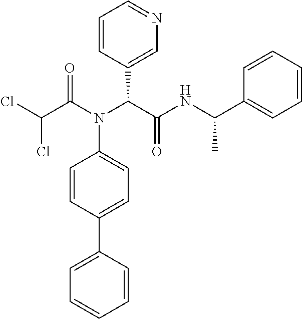 [SMILES: C[C@H](NC(=O)[C@@H](c1cccnc1)N(C(=O)C(Cl)Cl)c1ccc(-c2ccccc2)cc1)c1ccccc1]

N-([1,1′-biphenyl]-4-yl)-2,2-dichloro-N-((R)-2-oxo-2-(((S)-1-phenylethyl)amino)-1-(pyridin-3-yl)ethyl)acetamide (Jung-62-2R). White solid, 41% yield. ¹H NMR (500 MHz, DMSO-d6) δ8.65 (d, J=8.0 Hz, 1H), 8.33 (d, J=2.0 Hz, 1H), 8.28 (dd, J=4.5, 1.5 Hz, 1H), 7.83-6.73 (m, 16H), 6.07 (d, J=20.0 Hz, 2H), 4.95 (p, J=7.0 Hz, 1H), 1.18 (d, J=7.0 Hz, 3H). ¹³C NMR (125 MHz, DMSO-d6) δ167.2, 163.1, 151.2, 149.2, 143.7, 140.2, 138.5, 137.4, 136.1, 131.2, 130.0, 129.0, 128.2, 127.9, 127.0, 126.8, 126.6, 126.2, 123.1, 65.1, 62.5, 48.3, 22.1. C₂₉H₂₅Cl₂N₃O₂, HRMS calculated for m/z [M+H]⁺: 518.140208 (calculated), 518.13966 (found).

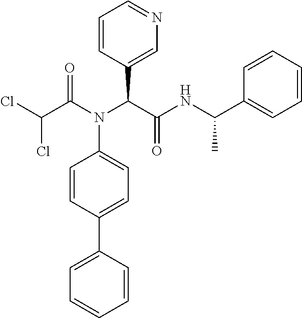 [SMILES: C[C@H](NC(=O)[C@H](c1cccnc1)N(C(=O)C(Cl)Cl)c1ccc(-c2ccccc2)cc1)c1ccccc1]

N-([1,1′-biphenyl]-4-yl)-2,2-dichloro-N-((S)-2-oxo-2-(((S)-1-phenylethyl)amino)-1-(pyridin-3-yl)ethyl)acetamide (Jung-62-2S). White solid, 40% yield. ¹H NMR (500 MHz, DMSO-d6) δ8.69 (d, J=7.5 Hz, 1H), 8.24 (dd, J=4.5, 1.5 Hz, 1H), 8.20 (d, J=2.0 Hz, 1H), 7.80-6.71 (m, 16H), 6.07 (s, 2H), 4.92 (p, J=7.0 Hz, 1H), 1.31 (d, J=7.0 Hz, 3H). ¹³C NMR (125 MHz, DMSO-d6) δ167.1, 163.1, 151.3, 149.1, 144.2, 140.2, 138.5, 137.4, 136.1, 131.2, 129.5, 128.9, 128.1, 127.9, 127.0, 126.6, 126.6, 125.7, 122.7, 65.1, 62.5, 48.5, 22.3. C₂₉H₂₅Cl₂N₃O₂, HRMS calculated for mlz [M+H]⁺: 518,140208 (calculated), 518.13966 (found).

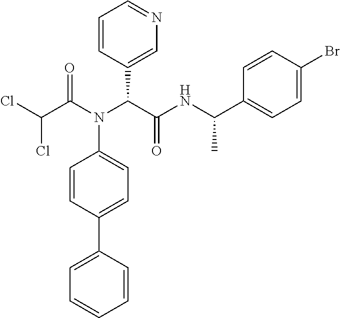 [SMILES: C[C@H](NC(=O)[C@@H](c1cccnc1)N(C(=O)C(Cl)Cl)c1ccc(-c2ccccc2)cc1)c1ccc(Br)cc1]

N-([1,1′-biphenyl]-4-yl)-N-((R)-2-(((S)-1-(4-bromophenypethyl)amino)-2-oxo-1-(pyridin-3-yl)ethyl)-2,2-dichloroacetamide (Jun9-90-3R). White solid, 35% yield. ¹H NMR (500 MHz, DMSO-d6) δ8.70 (d, J=7.5 Hz, 1H), 8.33 (s, 1H), 8.28 (d, J=4.5 Hz, 1H), 7.80-6.72 (m, 16H), 6.06 (d. J=5.5 Hz, 2H), 4.91 (p, J=7.0 Hz, 1H), 1.17 (d, J=7.0 Hz, 3H). ¹³C NMR (125 MHz, DMSO-d6) δ167.3, 163.1, 151.2, 149.2, 143.3, 140.2, 138.5, 137.4, 136.1, 131.1, 129.9, 129.0, 128.4, 128.0, 127.0, 126.6, 123.1, 119.8, 65.1, 62.6, 47.9, 22.0. C₂₉H₂₄BrCl₂N₃O₂, HRMS calculated for m/z [M+H]⁺: 596.050719 (calculated), 596.05017 (found).

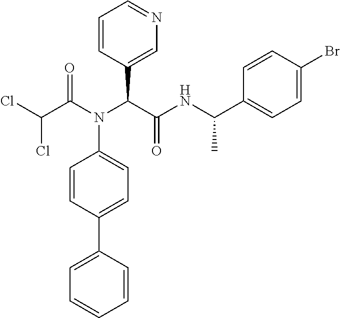 [SMILES: C[C@H](NC(=O)[C@H](c1cccnc1)N(C(=O)C(Cl)Cl)c1ccc(-c2ccccc2)cc1)c1ccc(Br)cc1]

N-([1,1′-biphenyl]-4-yl)-N-((S)-2-(((S)-1 -(4-bromophenyl)ethyl)amino)-2-oxo-1-(pyridin-3-yl)ethyl)-2,2-dichloroacetamide (Jun9-90-3S). White solid, 34% yield. ¹H NMR (500 MHz, DMSO-d6) δ8.73 (d, J=7.5 Hz, 1H), 8.25 (d, J=4.5 Hz, 1H), 8.19 (s, 1H), 7.83-6.71 (m, 16H), 6.06 (d, J=9.0 Hz, 2H), 4.89 (p, J=7.0 Hz, 1H), 1.28 (d, J=7.0 Hz, 3H). ¹³C NMR (125 MHz, DMSO-d6) δ167.2, 163.1, 151.2, 149.2, 143.8, 140.2, 138.5, 137.4, 136.0, 131.1, 131.0, 129.4, 128.9, 128.0, 127.9, 127.0, 126.6, 122.8, 119.6, 65.1, 62.5, 48.1, 22.1. C₂₉H₂₄BrCl₂N₃O₂, HRMS calculated for m/z [M+H]⁺: 596.050719 (calculated), 596.05017 (found).

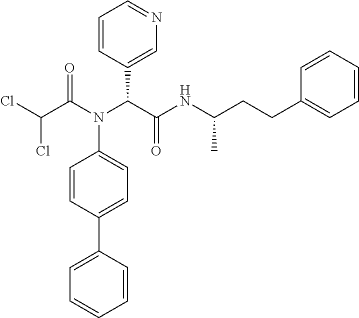 [SMILES: C[C@@H](CCc1ccccc1)NC(=O)[C@@H](c1cccnc1)N(C(=O)C(Cl)Cl)c1ccc(-c2ccccc2)cc1]

N-([1,1′-biphenyl]-4-yl)-2,2-dichloro-N-((R)-2-oxo-2-(((S)-4-phenylbutan-2-yl)amino)-1-(pyridin-3-yl)ethyl)acetamide (Jun9-90-4R). White solid, 32% yield. ¹H NMR (500 MHz, DMSO-d6) δ8.33 (s, 1H), 8.26 (d, J=4.0 Hz, 1H), 8.16 (d, J=8.0 Hz, 1H), 7.88-6.77 (m, 16H), 6.08 (s, 1H), 6.01 (s, 1H), 3.83-3.71 (m, 1H), 2.66-2.52 (m, 2H), 1.62 (dd, J=14.5, 7.5 Hz, 2H), 0.89 (d, J=6.5 Hz, 3H). ¹³C NMR (125 MHz, DMSO-d6) δ167.2, 163.1, 151.2, 149.1, 142.0, 140.2 138.5, 137.4, 136.2, 131.2, 130.0, 128.9, 128.5, 128.2, 127.9, 127.0, 126.6, 125.6, 123.0, 65.2, 62.7, 44.3, 37.8, 31.7, 20.5. C₃₁H₂₉Cl₂N₃O₂, HRMS calculated for m/z [M+H]⁺: 546.171508 (calculated), 546.17096 (found).

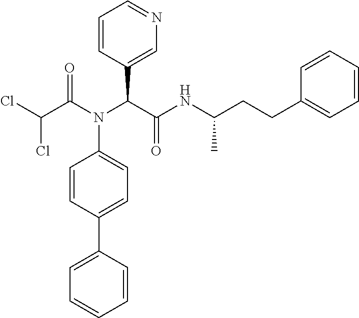 [SMILES: C[C@@H](CCc1ccccc1)NC(=O)[C@H](c1cccnc1)N(C(=O)C(Cl)Cl)c1ccc(-c2ccccc2)cc1]

N-([1,1′-biphenyl]-4-yl)-2,2-dichloro-N-((S)-2-oxo-2-(((S)-4-phenylbutan-2-yl)amino)-1-(pyridin-3-yl)ethyl)acetamide (Jun9-90-4S). White solid, 33% yield. ¹H NMR (500 MHz, DMSO-d6) δ8.34 (s, 1H), 8.27 (d, J=4.5 Hz, 1H), 8.15 (d, J=8.0 Hz, 1H), 7.92-6.70 (m, 16H), 6.07 (s, 1H), 5.99 (s, 1H), 3.84-3.72 (m, 1H), 2.28-2.20 (m, 2H), 1.59-1.43 (m, 2H), 1.05 (d, J=6.5 Hz, 3H). ¹³C NMR (125 MHz, DMSO-d6) δ167.2, 163.1, 151.2, 149.2, 141.7, 140.2, 138.5, 137.5, 136.2, 131.2, 130.1, 128.9, 128.2, 128.1, 127.9, 127.0, 126.6, 125.6, 122.9, 65.2, 62.8, 44.5, 31.5, 20.6. C₃₁H₂₉Cl₂N₃O₂, HRMS calculated for m/z [M+H]⁺: 546.171508 (calculated), 546.17096 (found).

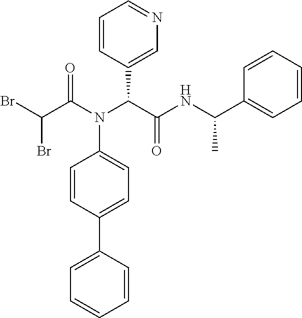 [SMILES: C[C@H](NC(=O)[C@@H](c1cccnc1)N(C(=O)C(Br)Br)c1ccc(-c2ccccc2)cc1)c1ccccc1]

N-([1,1′-biphenyl]-4-yl)-2,2-dibromo-N-((R)-2-oxo-2-(((S)-1-phenylethyl)amino)-1-(pyridin-3-yl)ethypacetamide (Jun9-89-2R). White solid, 37% yield. ¹H NMR (500 MHz, DMSO-d6) δ8.63 (d, J=8.0 Hz, 1H), 8.33 (s, 1H), 8.28 (d, J=4.5 Hz, 1H), 7.78-6.77 (m, 16H), 6.08 (s, 1H), 5.79 (s, 1H), 4.95 (p, J=7.0 Hz, 1H), 1.18 (d, J=7.0 Hz, 3H). ¹³C NMR (125 MHz, DMSO-d6) δ167.2, 163.6, 151.1, 149.1, 143.6, 140.2, 138.5, 137.5, 136.7, 130.9, 130.1, 129.0, 128.3, 128.0, 127.0, 126.7, 126.6, 126.2, 123.1, 62.5, 48.2, 36.2, 22.1. C₂₉H₂₅Br₂N₃O₂, HRMS calculated for m/z [M+H]⁺: 606.039174 (calculated), 606.03863 (found).

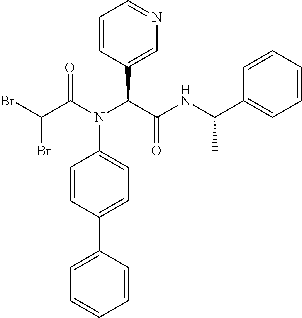 [SMILES: C[C@H](NC(=O)[C@H](c1cccnc1)N(C(=O)C(Br)Br)c1ccc(-c2ccccc2)cc1)c1ccccc1]

N-([1,1′-biphenyl]-4-yl)-2,2-dibromo-N-((S)-2-oxo-2-(((S)-1-phenylethyl)amino)-1-(pyridin-3-yl)ethyl)acetamide (Jun9-89-2S). White solid, 38% yield. ¹H NMR (500 MHz, DMSO-d6) δ8.67 (d, J=7.5 Hz, 1H), 8.24 (d, J=4.5 Hz, 1H), 8.19 (s, 1H), 7.82-6.66 (m, 16H), 6.04 (s, 1H), 5.80 (s, 1H), 4.97-4.84 (m, 1H), 1.30 (d, J=7.0 Hz, 3H). ¹³C NMR (125 MHz, DMSO-d6) δ167.2, 163.6, 151.2, 149.1, 143.6, 140.2, 138.5, 137.5, 136.7, 130.8, 130.2, 129.0, 128.3, 128.0, 127.1, 126.7, 126.6, 126.2, 123.1, 62.5, 48.2, 36.2, 22.1. C₂₉H₂₅Br₂N₃O₂, HRMS calculated for m/z [M+H]⁺: 606.039174 (calculated), 606.03863 (found).

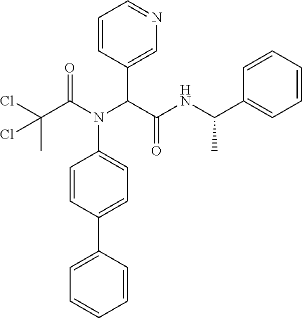 [SMILES: C[C@H](NC(=O)C(c1cccnc1)N(C(=O)C(C)(Cl)Cl)c1ccc(-c2ccccc2)cc1)c1ccccc1]

N-([1,1′-biphenyl]-4-yl)-2,2-dichloro-N-(2-oxo-2-(((S)-1-phenylethyl)amino)-1-(pyridin-3-yl)ethyl)propanamide (Jun9-76-4), dr=1:1. White solid, 79% yield. ¹H NMR (500 MHz, DMSO-d6) δ8.77-8.35 (m, 3H), 7.99-6.75 (m, 15H), 6.11 (s, 1H), 4.98-4.6 (m, 1H), 2.15 (s, 1.5H), 2.12 (s, 1.5H), 1.29 (d, J=7.0 Hz, 1.5H), 1.17 (d, J=7.0 Hz, 1.5H). ¹³C NMR (125 MHz, DMSO-d6) δ166.9, 166.8, 164.0, 148.7, 148.2, 146.2, 144.2, 143.7, 139.7, 138.6, 133.6, 132.1, 131.5, 128.9, 128.9, 128.3, 128.1, 127.9, 127.9, 126.8, 126.7, 126.6, 126.6, 126.2, 125.7, 125.5, 124.4, 124.0, 81.4, 81.3, 64.3, 64.3, 48.5, 48.3, 36.9, 22.3, 22.0. C₃₀H₂₇Cl₂N₃O₂, HRMS calculated for m/z [M+H]⁺: 532.155858 (calculated), 532.15531 (found).

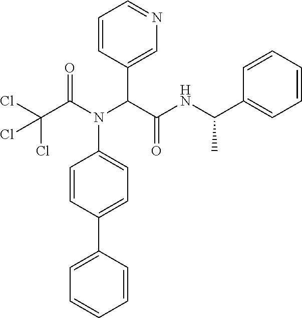 [SMILES: C[C@H](NC(=O)C(c1cccnc1)N(C(=O)C(Cl)(Cl)Cl)c1ccc(-c2ccccc2)cc1)c1ccccc1]

N-([1,1′-biphenyl]-4-yl)-2,2,2-trichloro-N-(2-oxo-2-(((S)-1-phenylethyl)amino)-1-(pyridin-3-yl)ethyl)acetamide (Jun9-72-4), dr=1:1. White solid, 74% yield. ¹H NMR (500 MHz, DMSO-d6) δ8.73 (dd, J=19.5, 7.5 Hz, 1H), 88.45-8.35 (m, 2H), 8.03-6.81 (m, 16H), 6.15 (s, 0.5H), 5.10-4.97 (m, 1H), 1.40 (d, J=7.0 Hz, 1.5H), 1.25 (d, J=7.0Hz, 1.5H). ¹³C NMR (125 MHz, DMSO-d6) δ167.2, 167.1, 159.4, 159.4, 151.7, 151.6, 149.1, 144.2, 143.7, 139.7, 139.7, 138.5, 138.5, 137.0, 137.7, 136.4, 133.5, 133.5, 129.8, 129.3, 128.9, 128.9, 128.2, 128.1, 127.9, 127.8, 126.8, 126.6, 126.5, 126.5, 126.1, 125.7, 122.9, 122.5, 92.9, 92.9, 65.6, 65.6, 48.5, 48.3, 22.3, 22.1. C₂₉H₂₄Cl₃N₃O₂, HRMS calculated for m/z [M+H]⁺: 552.101236 (calculated), 552.10069 (found).

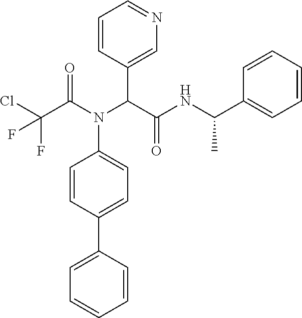 [SMILES: C[C@H](NC(=O)C(c1cccnc1)N(C(=O)C(F)(F)Cl)c1ccc(-c2ccccc2)cc1)c1ccccc1]

N-([1,1′-biphenyl]-4-yl)-2-chloro-2,2-difluoro-N-(2-oxo-2-(((S)-1-phenylethyl)amino)-1-(pyridin-3-yl)ethyl)acetamide (Jun9-77-2), dr=1:1. White solid, 60% yield. ¹H NMR (500 MHz, CDCl₃) δ8.41-8.15 (m, 2H), 7.76-6.81 (m, 15H), 6.55-6.35 (m, 2H), 5.88 (d, J=7.5 Hz, 1H), 5.11-5.03 (m, 1H), 1.42 (d, J=7.0 Hz, 1.5H), 1.34 (d, J=7.0 Hz, 1.5H). ¹³C NMR (125 MHz, CDCl₃) δ166.6, 166.4, 151.5, 151.5, 150.3, 150.3, 142.7, 142.4, 142.1, 141.9, 139.4, 138.2, 138.0, 135.0, 131.7, 131.4, 128.9, 128.9, 128.8, 128.6, 128.0, 127.9, 127.6, 127.4, 127.3, 127.1, 127.1, 126.7, 126.2, 125.9, 123.3, 123.2, 64.5, 64.2, 49.7, 49.6, 21.9, 21.5. C₂₉H₂₄ClF₂N₃O₂, HRMS calculated for m/z [M+H]⁺: 520.160336 (calculated), 520.15979 (found).

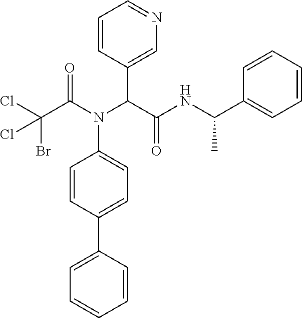 [SMILES: C[C@H](NC(=O)C(c1cccnc1)N(C(=O)C(Cl)(Cl)Br)c1ccc(-c2ccccc2)cc1)c1ccccc1]

N-([1,1′-biphenyl]-4-yl)-2-bromo-2,2-dichloro-N-(2-oxo-2-(((S)-1-phenylethyl)amino)-1-(pyridin-3-yl)ethyl)acetamide (Jun9-89-3). White solid, 35% yield, dr =1:1. ¹H NMR (500 MHz, DMSO-d6) δ8.82 (d, J=12.5 Hz, 1H), 8.69 (d, J=4.5 Hz, 0.5H), 8.56 (d, J=5.0 Hz, 0.5H), 8.08 (d, J=8.2 Hz, 0.5H), 7.87 (d, J=8.1 Hz, 0.5H), 7.53-7.10 (m, 16H), 6.30 (s, 0.5H), 6.22 (s, 0.5H), 5.15-5.07 (m, 1H), 1.53 (d, J=7.0 Hz, 1.5H), 1.50 (d, J=7.0 Hz, 1.5H). ¹³C NMR (125 MHz, DMSO-d6) δ165.6, 165.4, 161.9, 161.6, 145.6, 145.5, 145.1, 143.3, 143.0, 142.7, 142.7, 142.5, 142.3, 139.0, 136.3, 135.8, 133.2, 133.1, 132.7, 132.5, 129.1, 129.0, 128.8, 128.7, 128.3, 128.2, 127.7, 127.6, 127.1, 127.0, 126.8, 126.3, 126.0, 125.2, 125.2, 65.7, 65.0, 50.2, 50.0, 21.9, 21.5. C₂₉H₂₄BrCl₂N₃O₂, HRMS calculated for m/z [M+H]⁺: 596.050719 (calculated), 596.05017 (found).

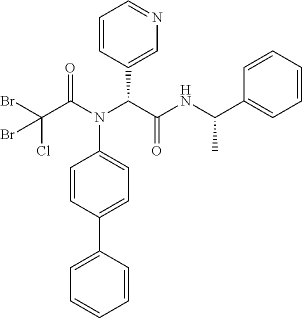 [SMILES: C[C@H](NC(=O)[C@@H](c1cccnc1)N(C(=O)C(Cl)(Br)Br)c1ccc(-c2ccccc2)cc1)c1ccccc1]

N-([1,1′-biphenyl]-4-yl)-2,2-dibromo-2-chloro-N-((R)-2-oxo-2-(((S)-1-phenylethyl)amino)-1-(pyridin-3-yl)ethyl)acetamide (Jun9-89-4R). White solid, 38% yield. ¹H NMR (500 MHz, CDCl₃) δ8.35 (d, J=4.5 Hz, 2H), 7.63-6.66 (m, 16H), 6.26 (d, J=7.5 Hz, 1H), 5.89 (s, 1H), 5.07 (p, J=7.0 Hz, 1H), 1.34 (d, J=7.0 Hz, 2H). ¹³C NMR (125 MHz, CDCl₃) δ166.8, 161.1, 151.6, 150.2, 142.5, 141.9, 139.5, 138.2, 137.0, 132.8, 129.5, 128.9, 128.8, 127.9, 127.6, 127.1, 126.3, 123.2, 67.0, 55.2, 49.6, 21.5. C₂₉H₂₄Br₂ClN₃O₂, HRMS calculated for m/z [M+H]⁺: 640.000202 (calculated), 639.99966 (found).

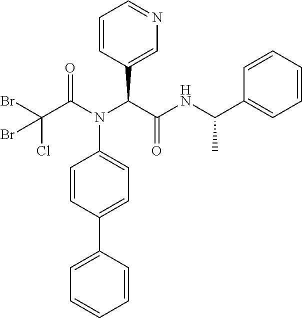 [SMILES: C[C@H](NC(=O)[C@H](c1cccnc1)N(C(=O)C(Cl)(Br)Br)c1ccc(-c2ccccc2)cc1)c1ccccc1]

N-([1,1′-biphenyl]-4-yl)-2,2-dibromo-2-chloro-N-((S)-2-oxo-2-(((S)-1-phenylethyl)amino)-1-(pyridin-3-yl)ethyl)acetamide (Jun9-89-4S). White solid, 40% yield. ¹H NMR (500 MHz, CDCl₃) δ8.31 (s, 1H), 8.25 (d, J=4.0 Hz, 1H), 7.90-6.53 (m, 16H), 6.28 (d, J=7.5 Hz, 1H), 5.92 (s, 1H), 5.03 (p, J=7.0 Hz, 1H), 1.39 (d, J=7.0 Hz, 3H). ¹³C NMR (125 MHz, CDCl₃) δ167.1, 161.1, 151.6, 150.1, 142.9, 141.7, 139.4, 137.9, 136.8, 133.1, 129.1, 128.8, 128.6, 127.9, 127.3, 127.0, 125.9, 123.0, 67.2, 55.2, 49.7, 22.1. C₂₉H₂₄Br₂ClN₃O₂, HRMS calculated for m/z [M+H]⁺: 640.000202 (calculated), 639.99966 (found).

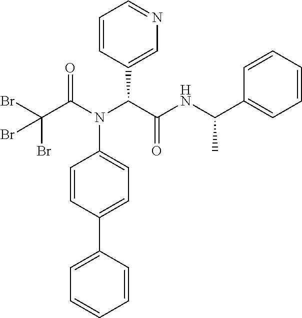 [SMILES: C[C@H](NC(=O)[C@@H](c1cccnc1)N(C(=O)C(Br)(Br)Br)c1ccc(-c2ccccc2)cc1)c1ccccc1]

N-([1,1′-biphenyl]-4-yl)-2,2,2-tribromo-N-((R)-2-oxo-2-(((S)-1-phenylethyl)amino)-1-(pyridin-3-yl)ethyl)acetamide (Jun9-88-6R). White solid, 35% yield. ¹H NMR (500 MHz, DMSO-d6) δ8.65 (d, J=8.0 Hz, 1H), 8.40 (d, J=1.5 Hz, 1H), 8.33 (dd, J=4.5, 1.5 Hz, 1H), 8.13-6.86 (m, 16H), 6.18 (s, 1H), 5.03 (p, J=7.0 Hz, 1H), 1.23 (d, J=7.0 Hz, 3H). ¹³C NMR (125 MHz, DMSO-d6) δ167.4, 159.4, 151.5, 149.1, 143.7, 139.6, 138.6, 137.6, 133.8, 130.2, 128.9, 128.2, 127.8, 126.8, 126.5, 126.2, 125.2, 122.9, 114.5, 66.1, 48.2, 34.6, 22.1. C₂₉H₂₄Br₃N₃O₂, HRMS calculated for m/z [M+H]⁺: 683.949685 (calculated), 683.94919 (found).

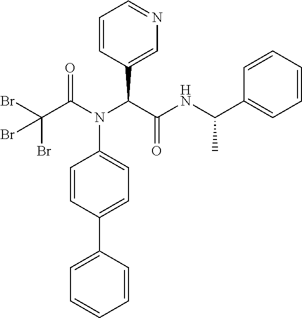 [SMILES: C[C@H](NC(=O)[C@H](c1cccnc1)N(C(=O)C(Br)(Br)Br)c1ccc(-c2ccccc2)cc1)c1ccccc1]

N-([1,1′-biphenyl]-4-yl)-2,2,2-tribromo-N-((S)-2-oxo-2-(((S)-1-phenylethyl)amino)-1-(pyridin-3-yl)ethyl)acetamide (Jun9-88-6S). White solid, 35% yield. ¹H NMR (500 MHz, DMSO-d6) δ8.63 (d, J=7.5 Hz, 1H), 8.29-8.18 (m, 2H), 7.99-6.77 (m, 16H), 6.08 (s, 1H), 4.92 (p, J=7.0 Hz, 1H), 1.31 (d, J=7.0 Hz, 3H). ¹³C NMR (125 MHz, DMSO-d6) δ167.3, 159.4, 151.5, 148.9, 144.3, 139.6, 138.5, 137.8, 137.3, 133.8, 129.8, 128.9, 128.1, 127.8, 126.6, 126.5, 125.7, 125.1, 122.6, 66.0, 48.4, 34.5, 22.3. C₂₉H₂₄Br₃N₃O₂, HRMS calculated for m/z [M+H]⁺: 683.949685 (calculated), 683.94919 (found).

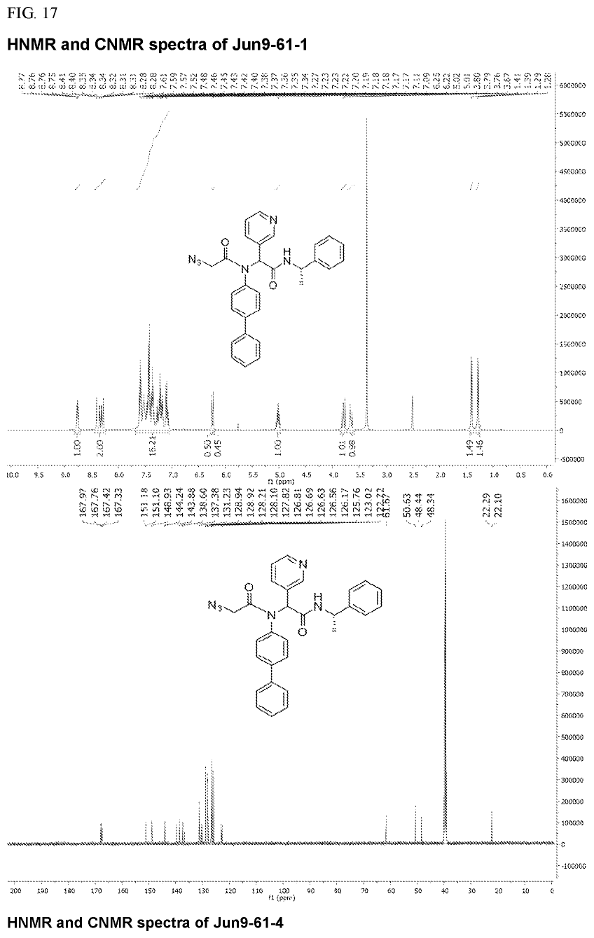

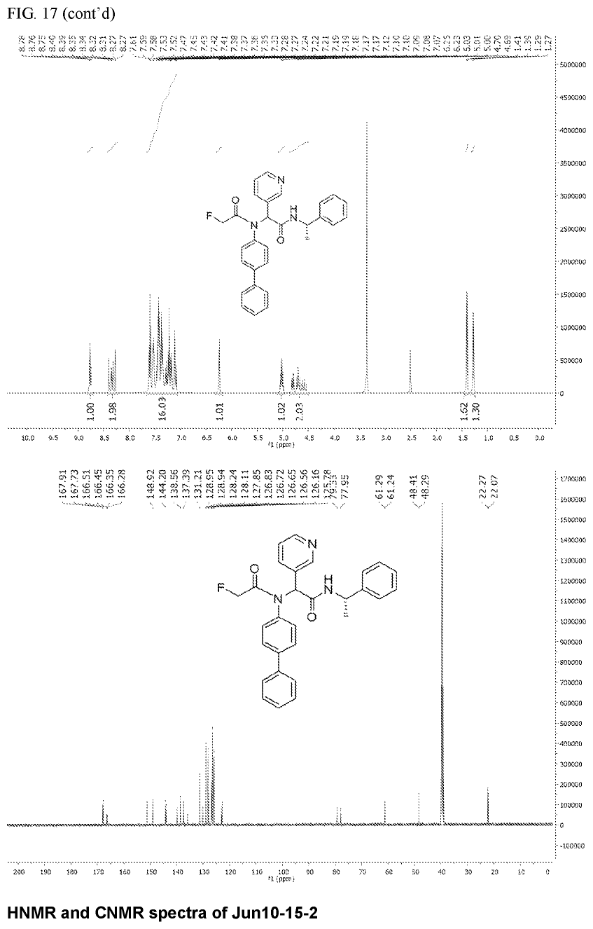

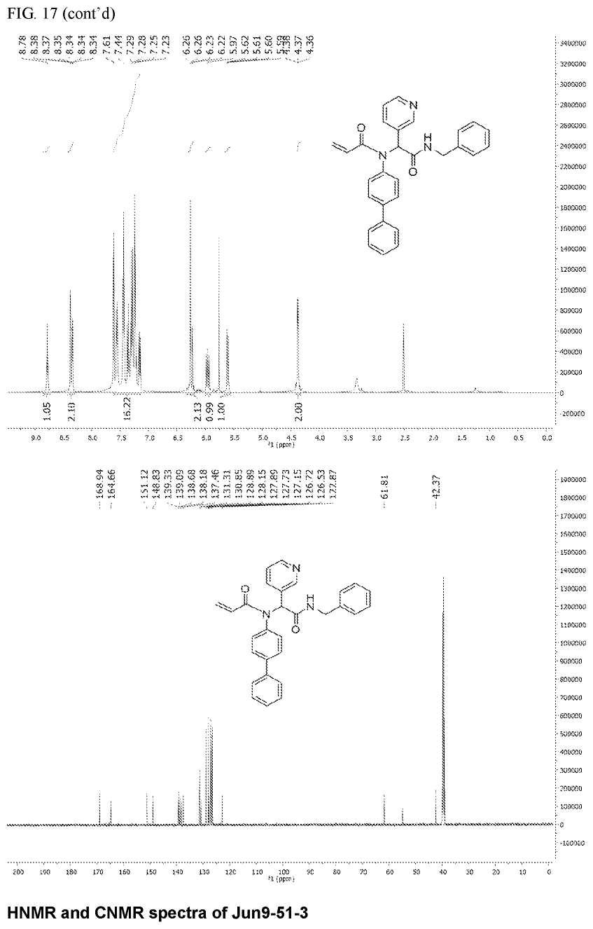

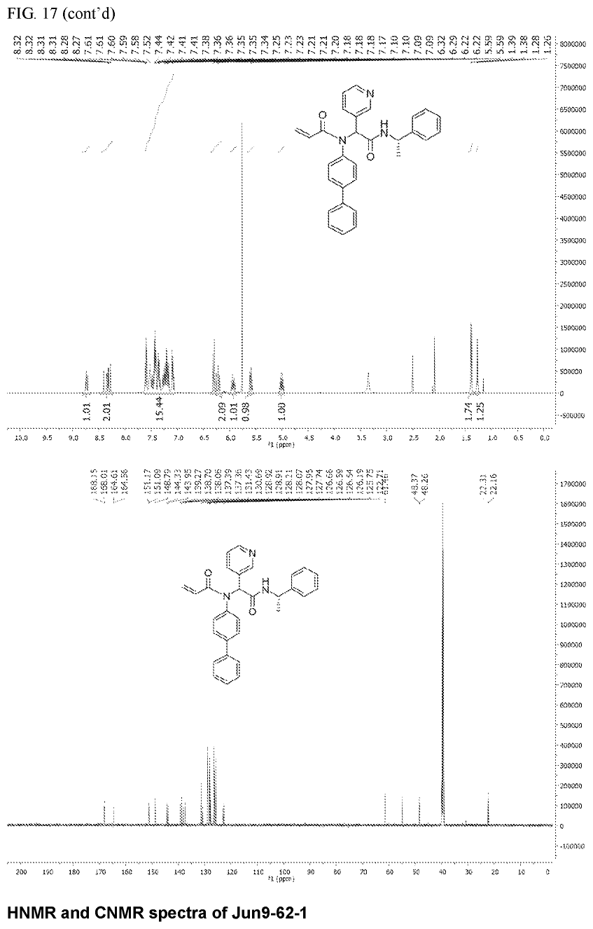

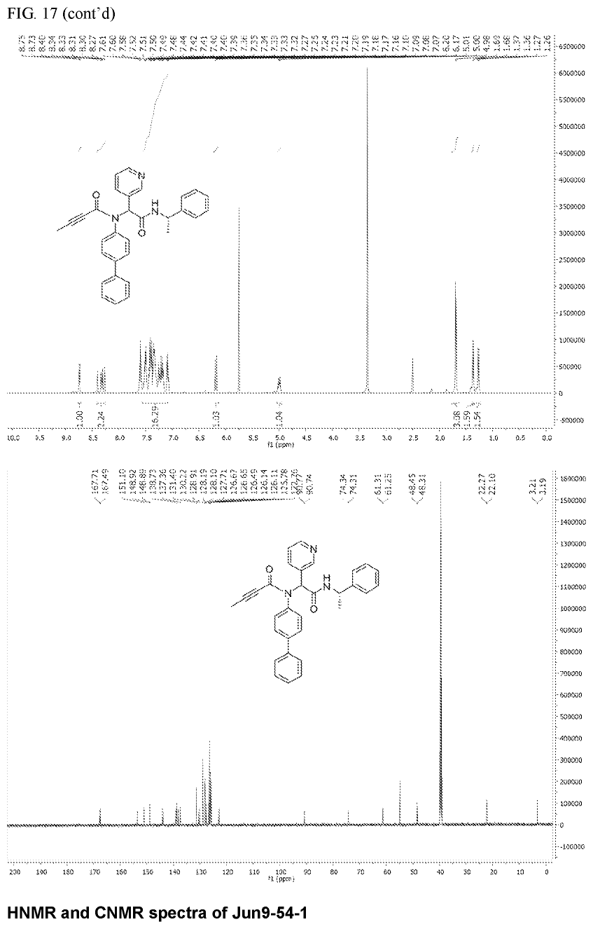

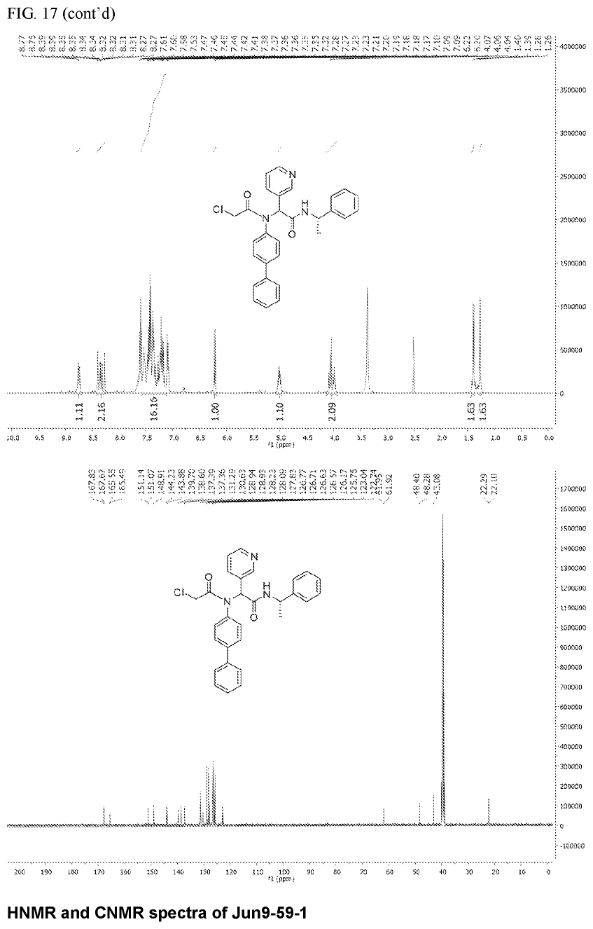

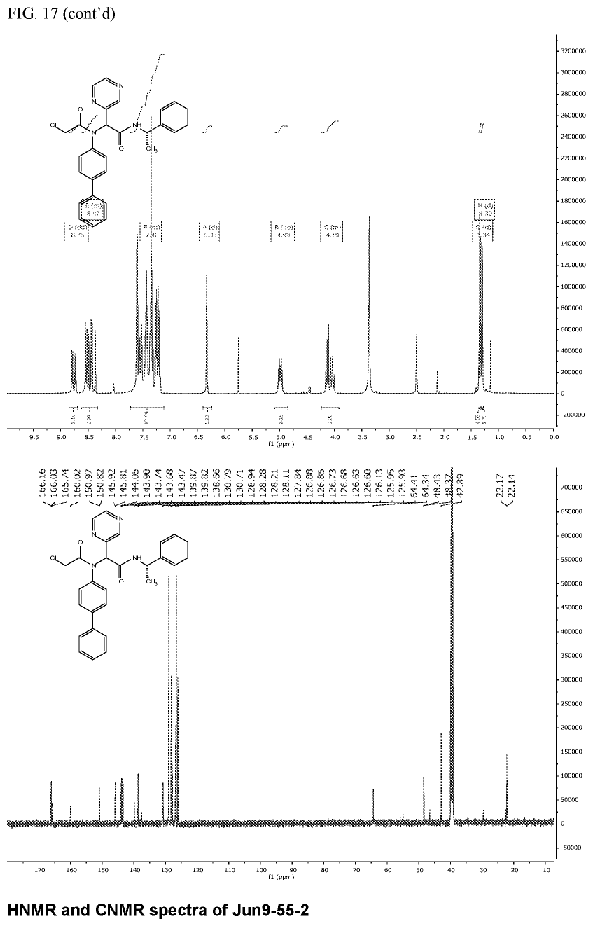

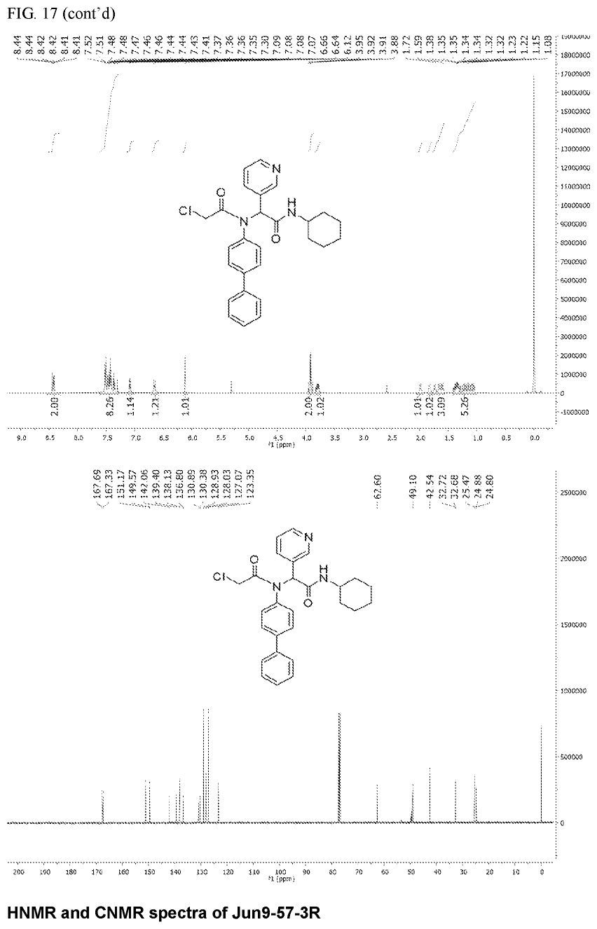

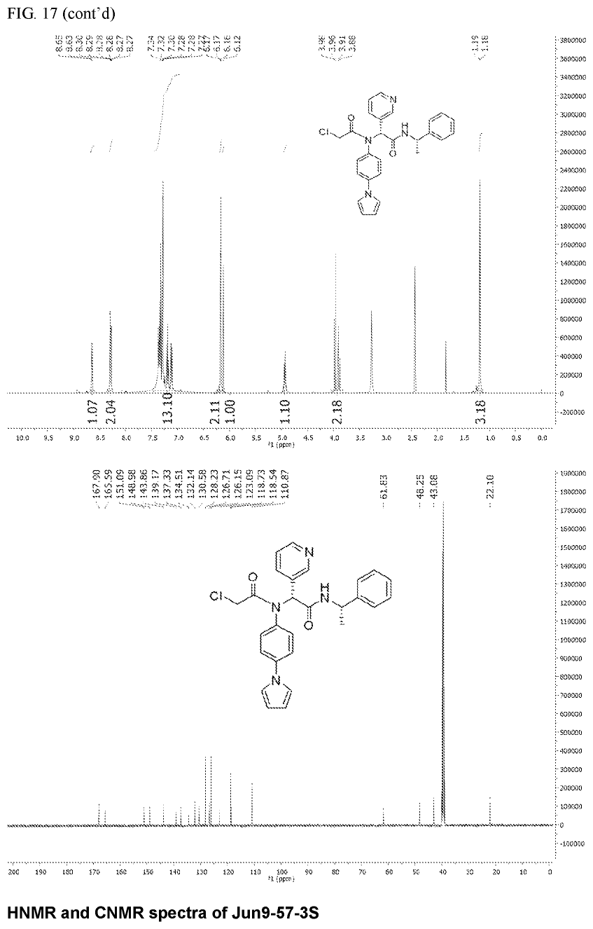

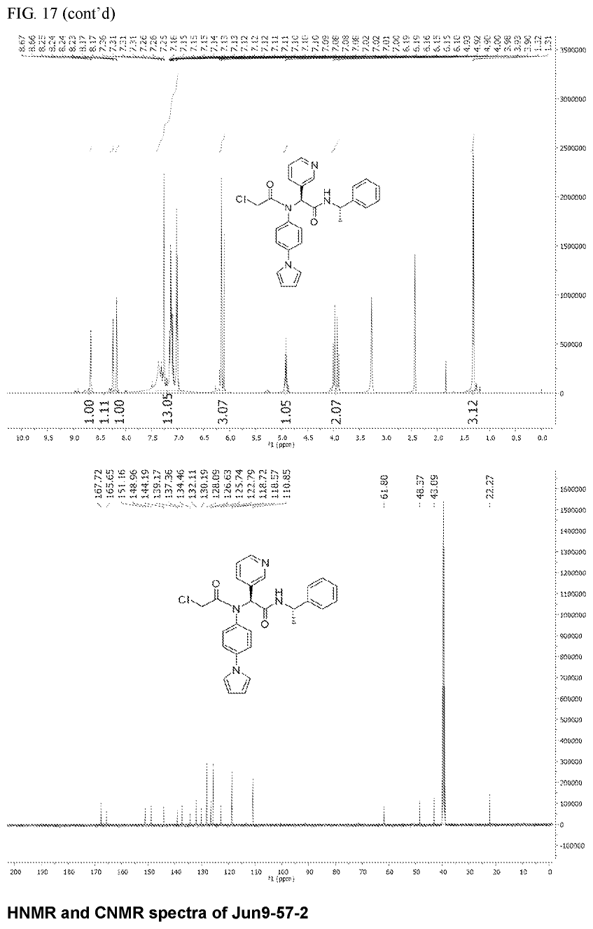

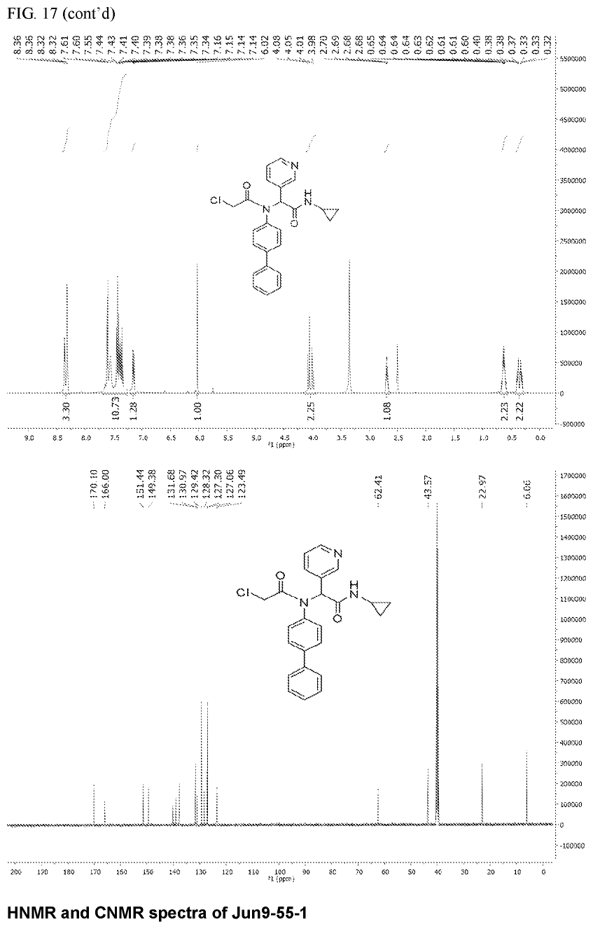

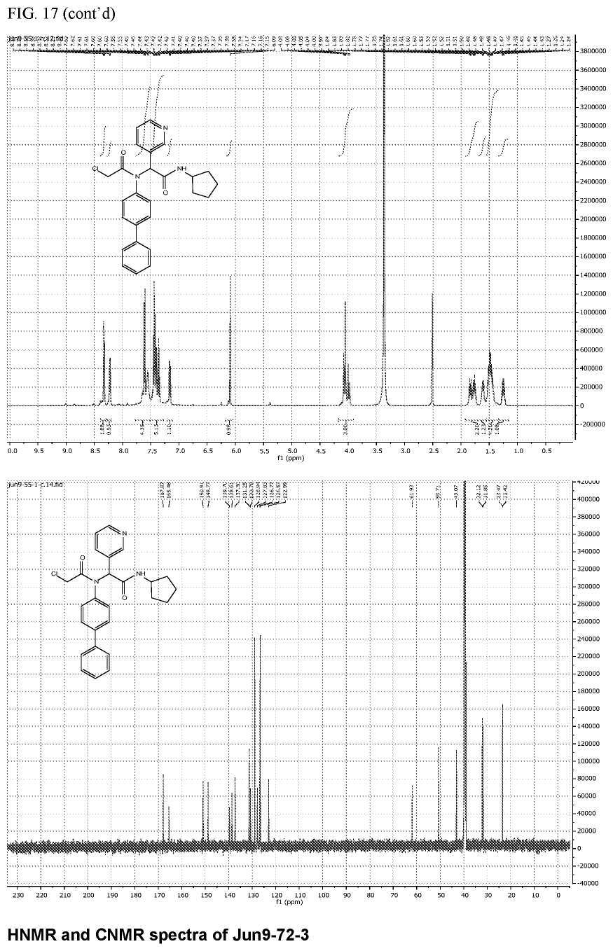

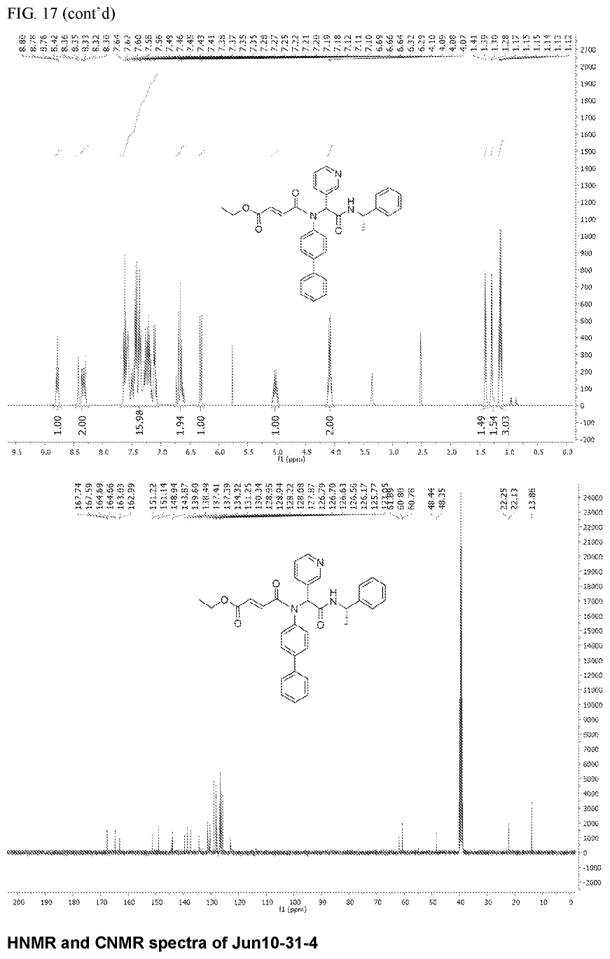

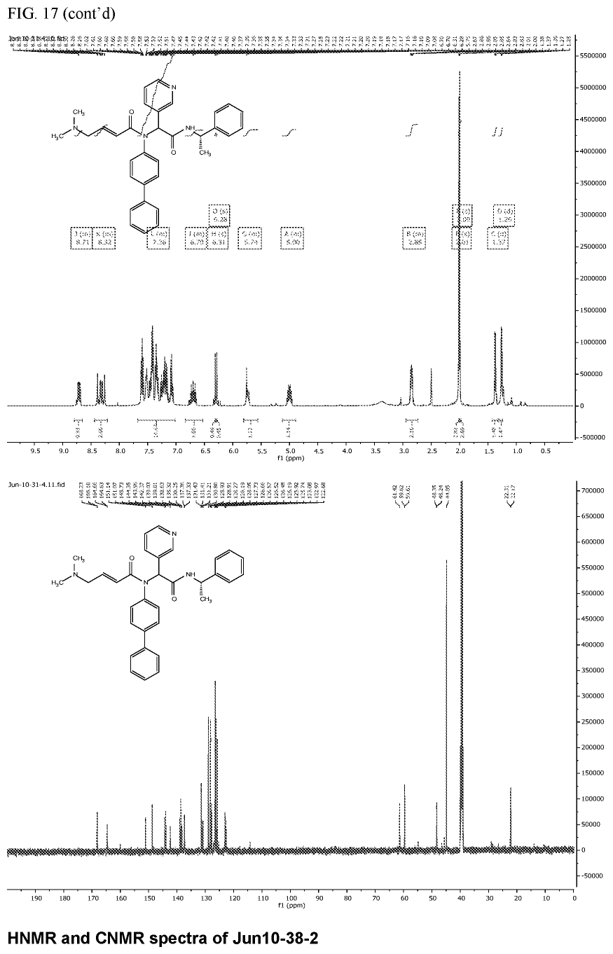

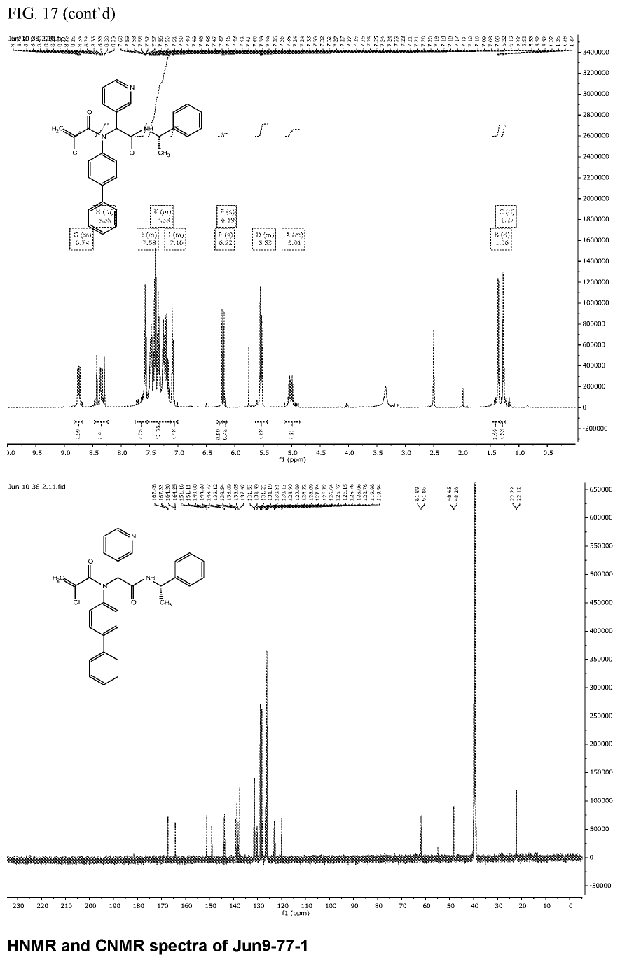

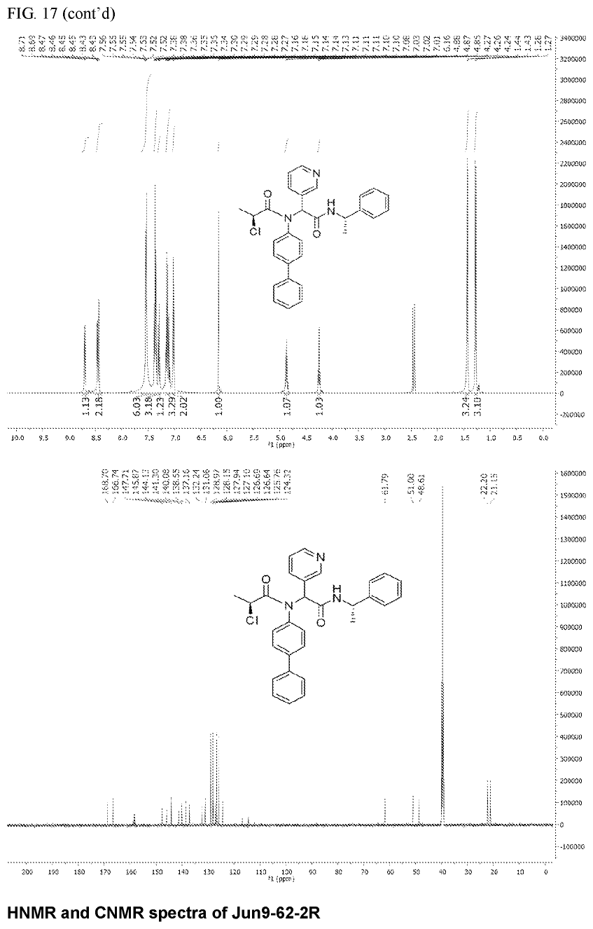

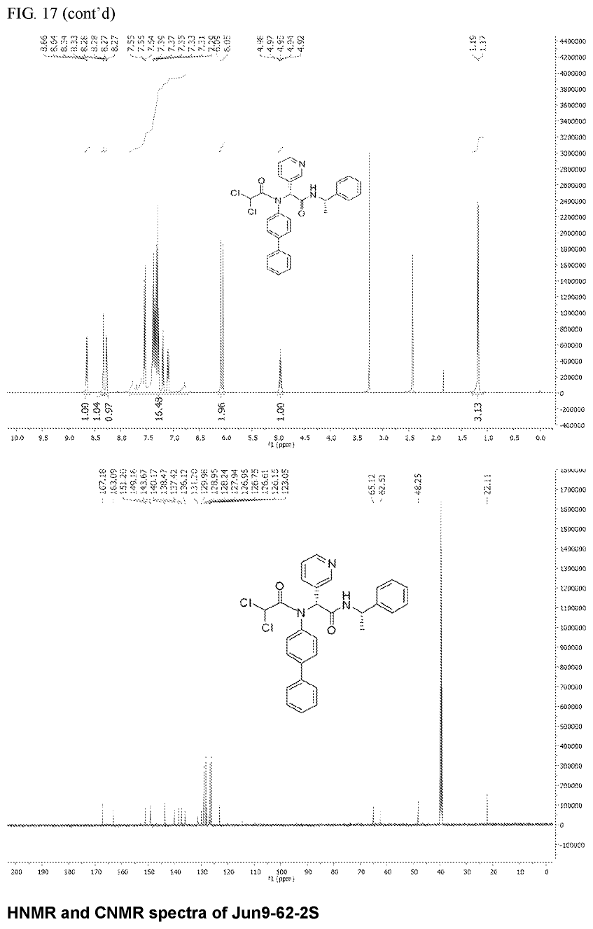

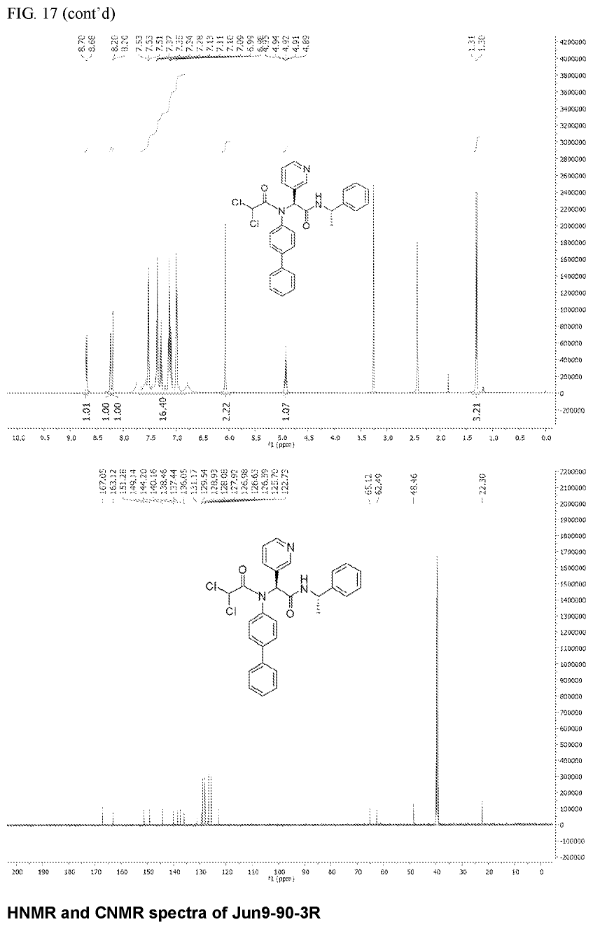

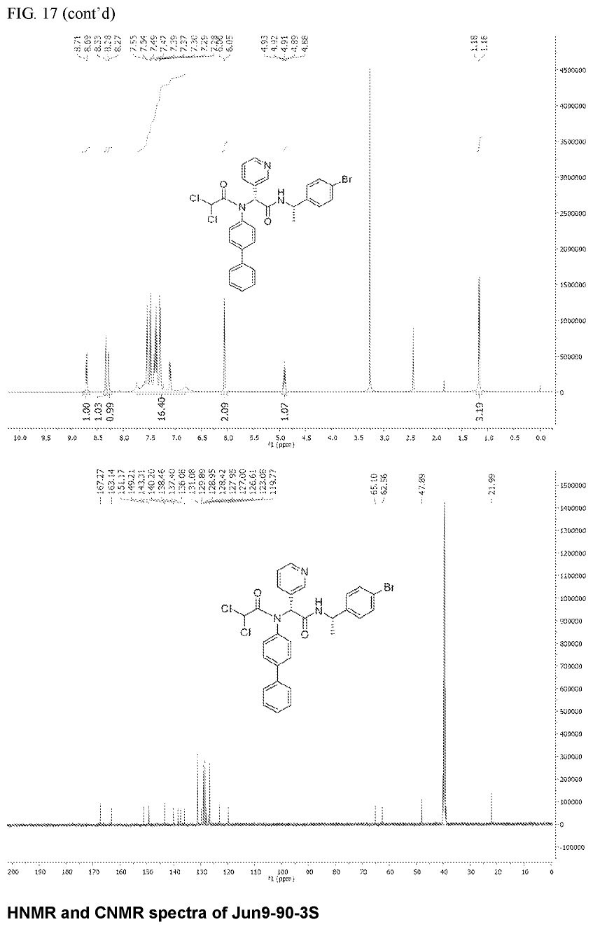

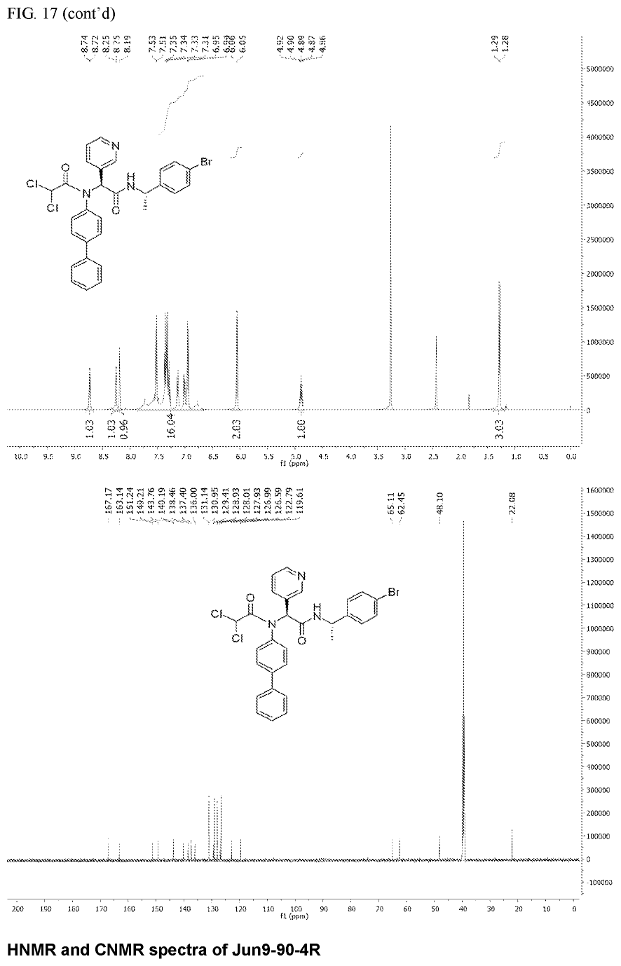

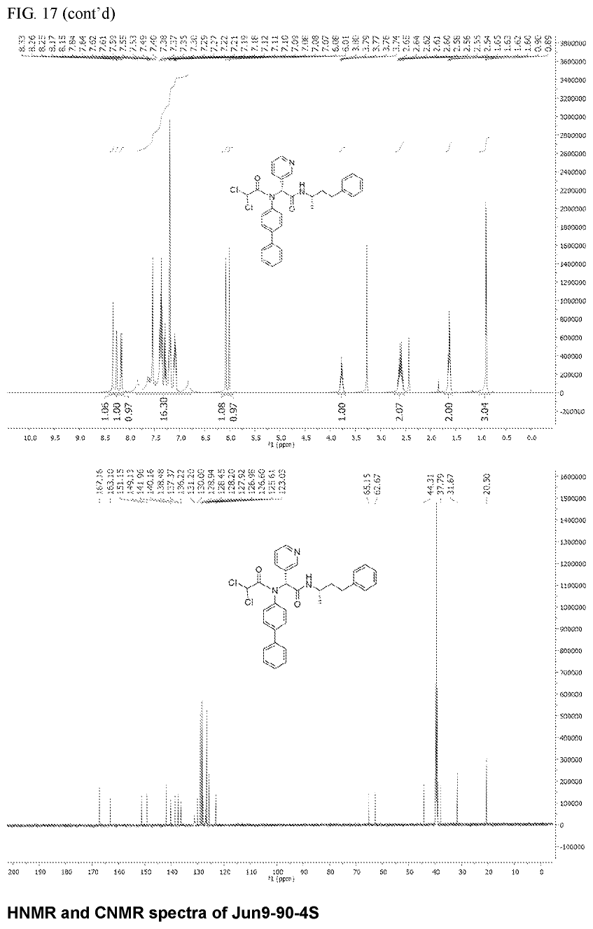

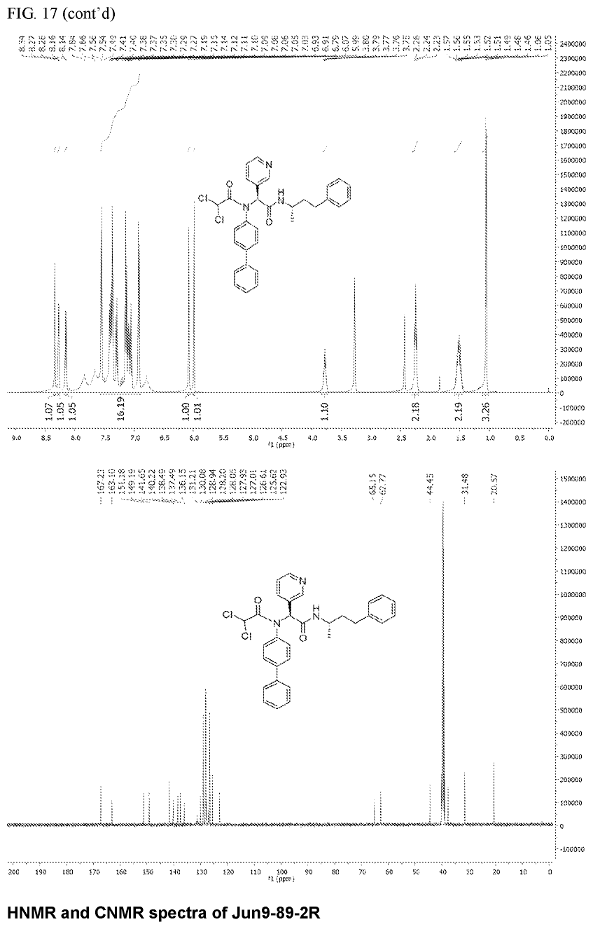

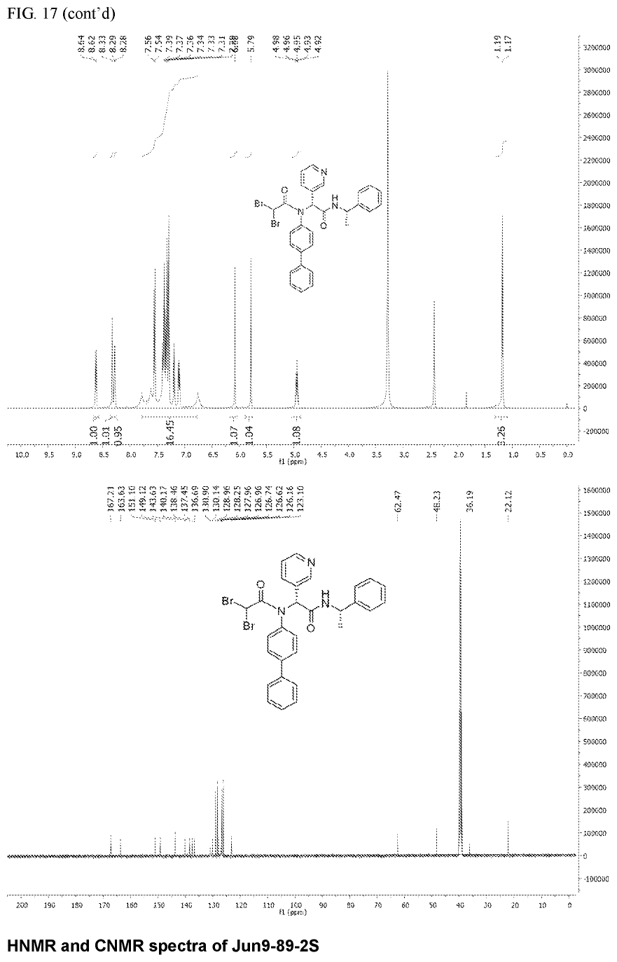

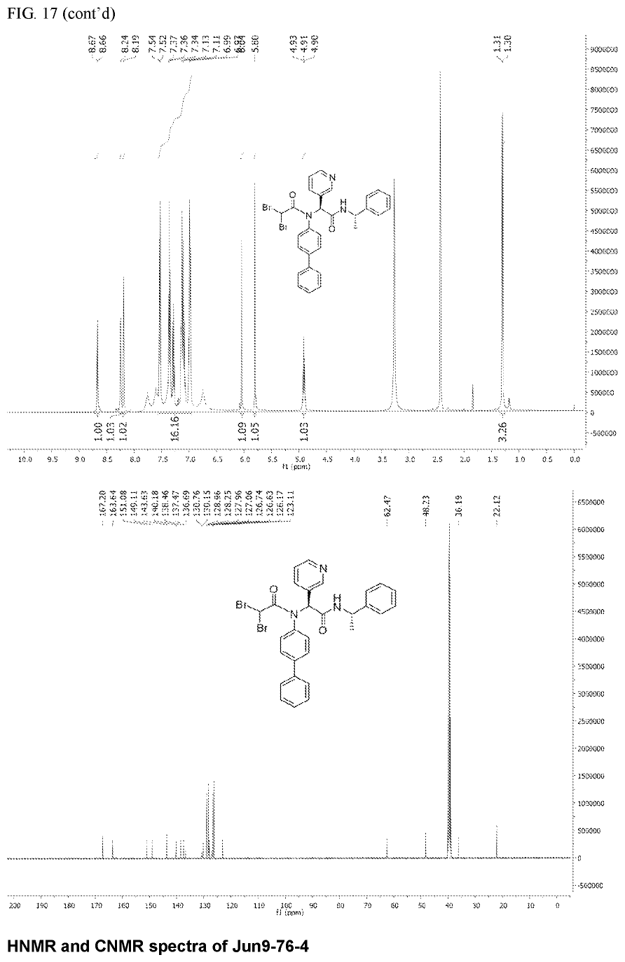

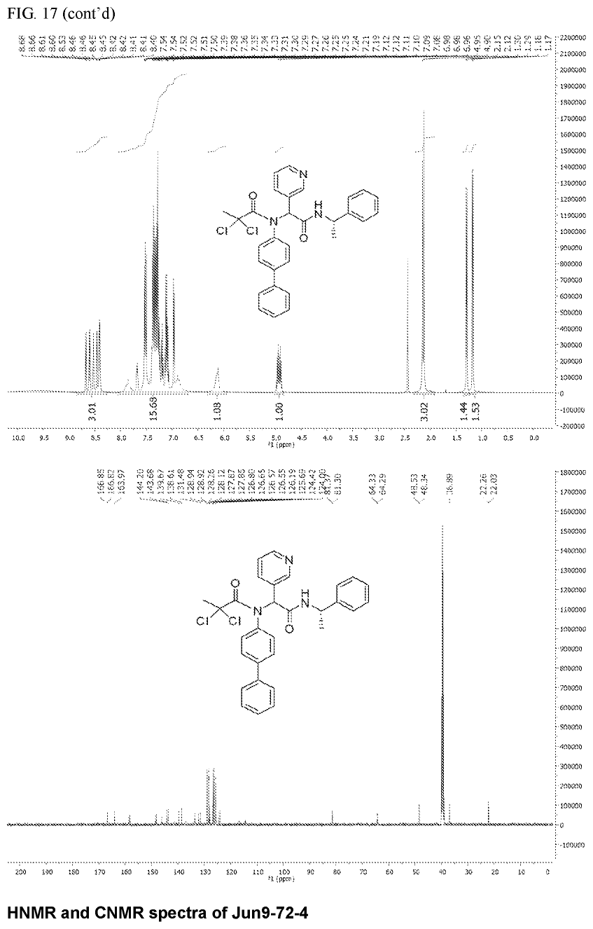

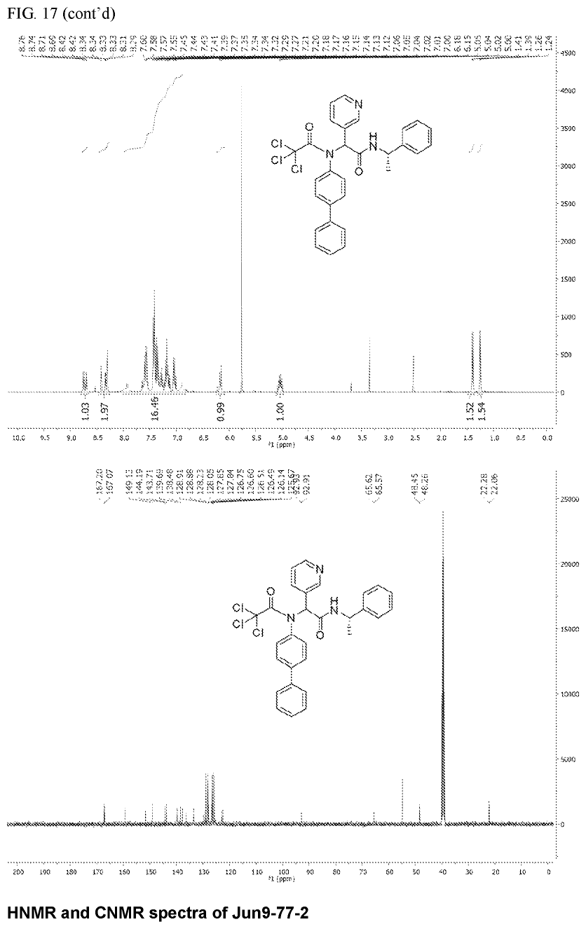

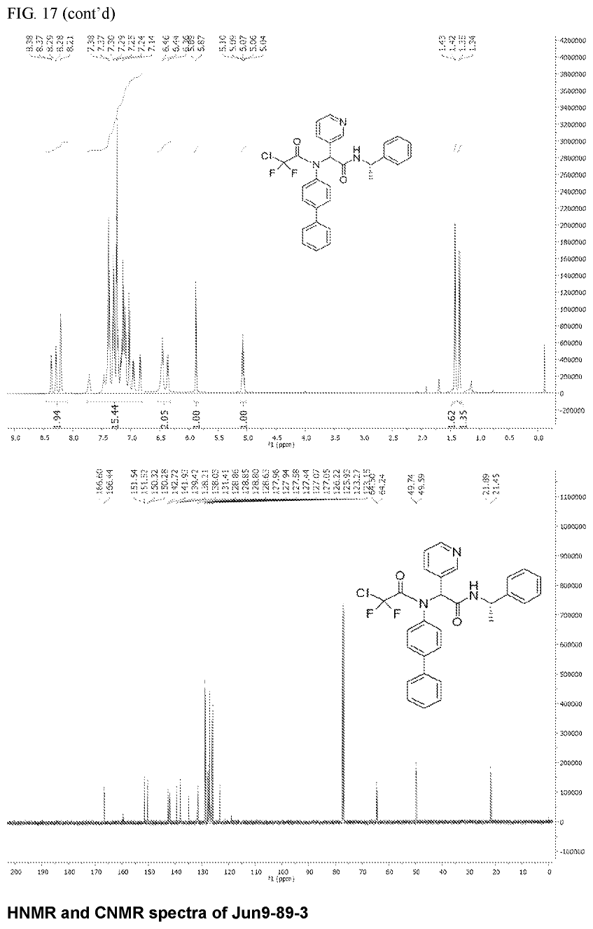

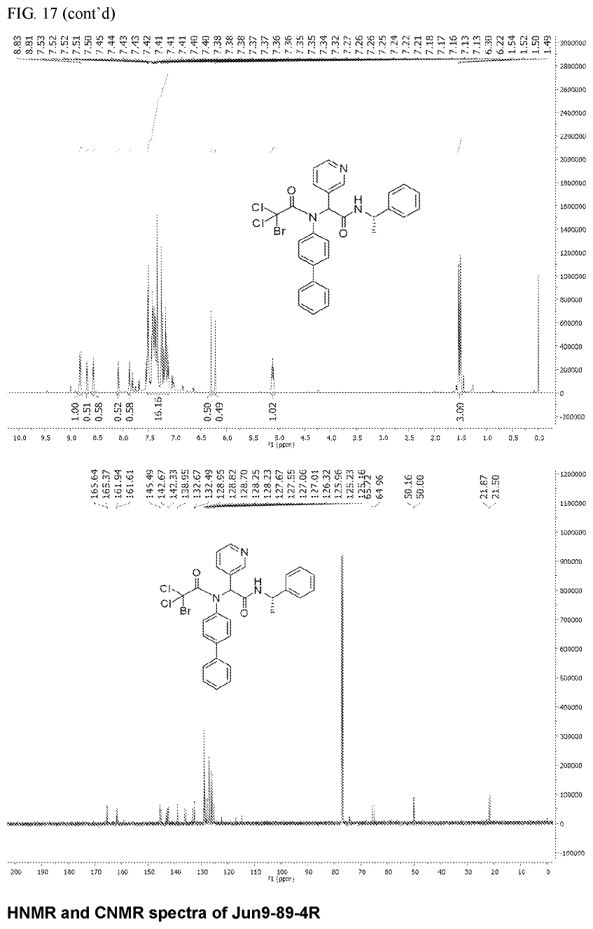

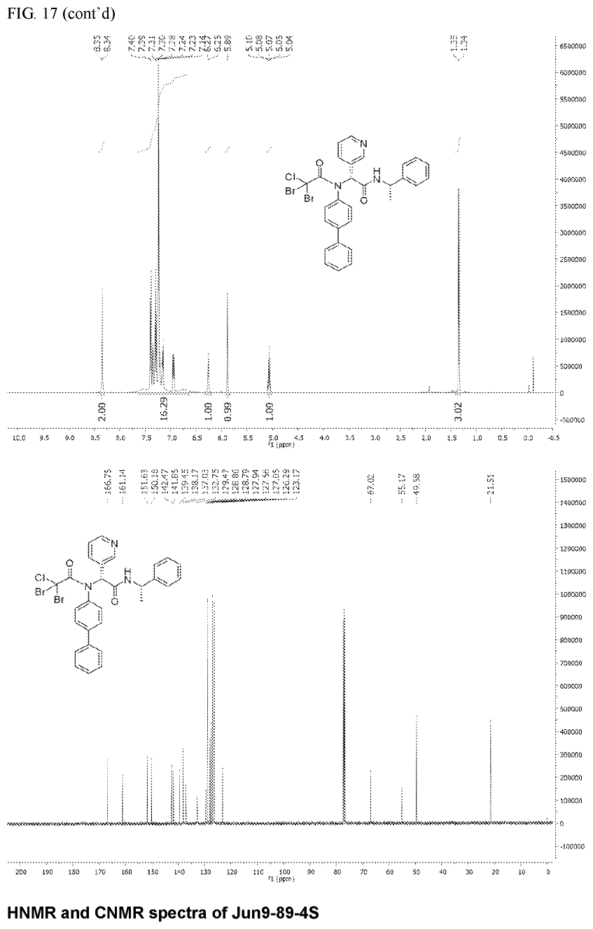

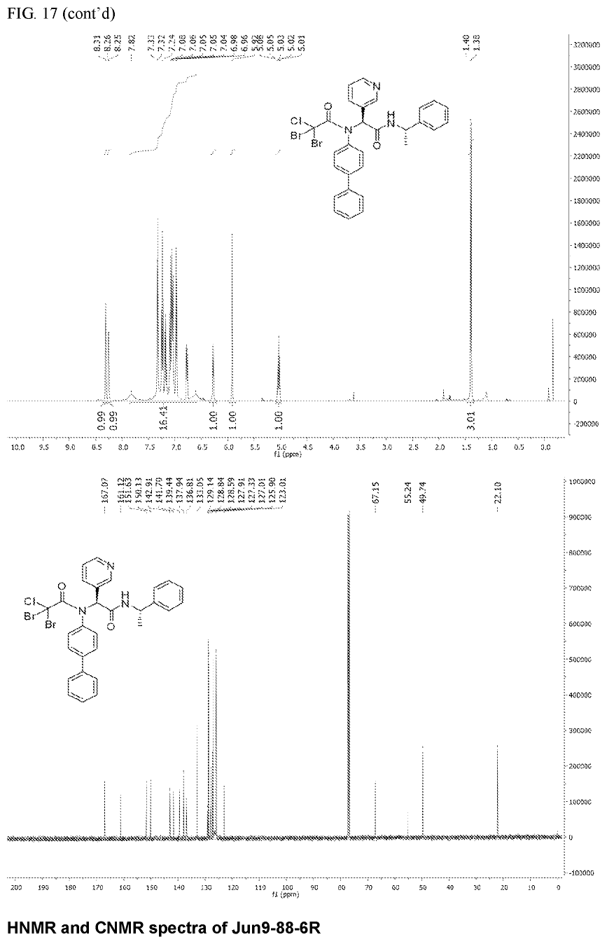

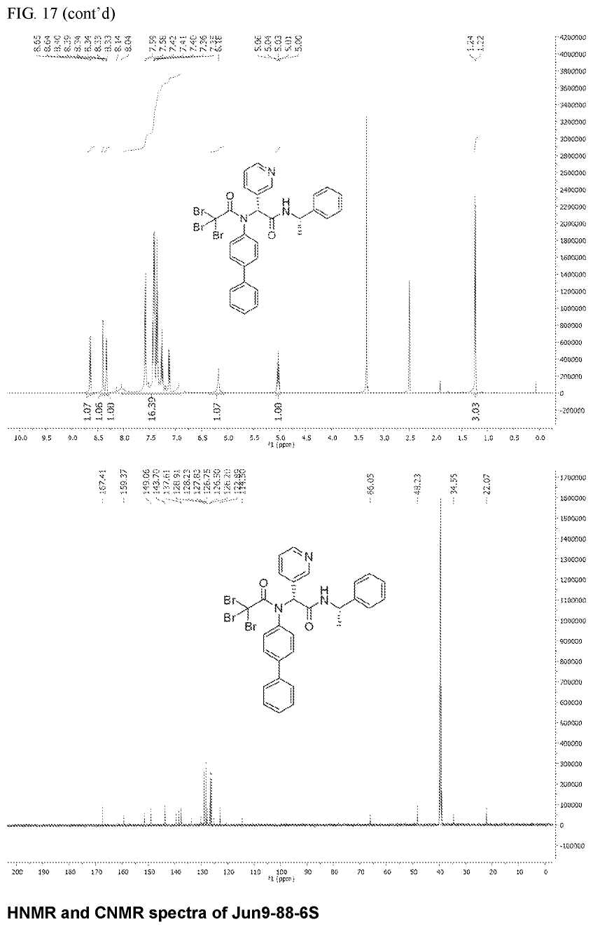

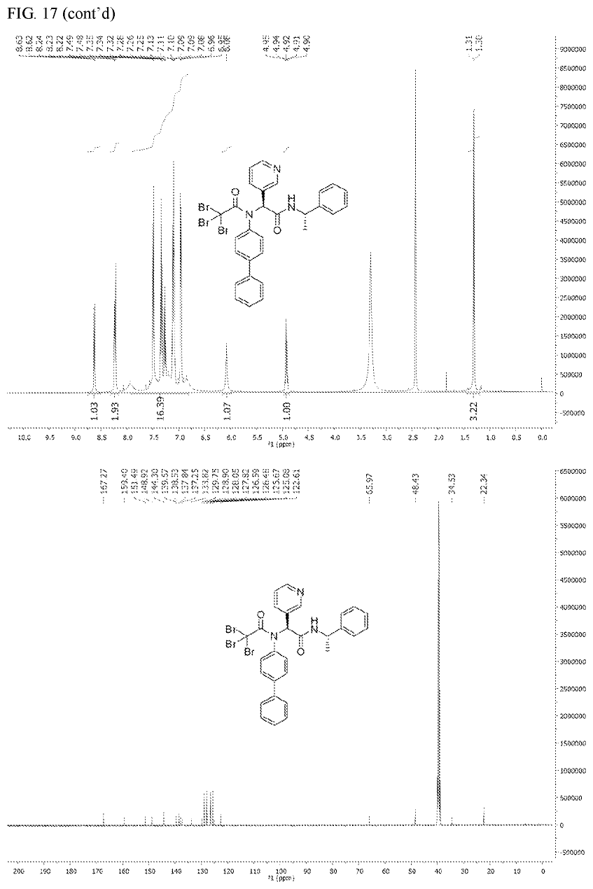

FIG. 17 shows HMNR and CNMR spectra of compounds described in Examples VI and VII.

FIG. 18 shows IC₅₀ values of additional compounds of the invention against M^{pro} protease activity.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. The following references cited herein are further incorporated by reference in their entireties:
1 Rut, W. et al. SARS-CoV-2 Mpro inhibitors and activity-based probes for patient-sample imaging. Nature Chemical Biology, doi:10.1038/s41589-020-00689-z (2020).
2 Boras, B. et al. Discovery of a Novel Inhibitor of Coronavirus 3CL Protease as a Clinical Candidate for the Potential Treatment of COVID-19. bioRxiv, 2020.2009.2012.293498, doi:10.1101/2020.09.12.293498 (2020).
3 Ma, C. L. et al. Boceprevir, GC-376, and calpain inhibitors II, XII inhibit SARS-CoV-2 viral replication by targeting the viral main protease. Cell Research 30, 678-692, doi:10.1038/s41422-020-0356-z (2020).
4 Sacco, M. D. et al. Structure and inhibition of the SARS-CoV-2 main protease reveals strategy for developing dual inhibitors against Mpro and cathepsin L. Science Advances, eabe0751, doi:10.1126/sciadv.abe0751 (2020).
5 Rathnayake, A. D. et al. 3C-like protease inhibitors block coronavirus replication in vitro and improve survival in MERS-CoV-infected mice. Sci Transl Med 12, eabc5332, doi:10.1126/scitranslmed.abc5332 (2020).
6 Pedersen, N. C. et al. Efficacy of a 3C-like protease inhibitor in treating various forms of acquired feline infectious peritonitis. J Feline Med Surg 20, 378-392, doi:10.1177/1098612X17729626 (2018).
7 Kim, Y. et al. Reversal of the Progression of Fatal Coronavirus Infection in Cats by a Broad-Spectrum Coronavirus Protease Inhibitor. PLoS Pathog 12, e1005531, doi:10.1371/journal.ppat.1005531 (2016).
8 Jacobs, J. et al. Discovery, synthesis, and structure-based optimization of a series of N-(tert-butyl)-2-(N-arylamido)-2-(pyridin-3-yl) acetamides (ML188) as potent noncovalent small molecule inhibitors of the severe acute respiratory syndrome coronavirus (SARS-CoV) 3CL protease. J Med Chem 56, 534-546, doi:10.1021/jm301580n (2013).
9 Cady, S. D., Wang, J., Wu, Y., DeGrado, W. F. & Hong, M. Specific binding of adamantane drugs and direction of their polar amines in the pore of the influenza M2 transmembrane domain in lipid bilayers and dodecylphosphocholine micelles determined by NMR spectroscopy. Journal of the American Chemical Society 133, 4274-4284, doi:10.1021/ja102581n (2011).
10 Marty, M. T. et al. Bayesian deconvolution of mass and ion mobility spectra: from binary interactions to polydisperse ensembles. Analytical chemistry 87, 4370-4376, doi:10.1021/acs.analchem.5b00140 (2015).
11 Repetto, G., del Peso, A. & Zurita, J. L. Neutral red uptake assay for the estimation of cell viability/cytotoxicity. Nat Protoc 3, 1125-1131, doi:10.1038/nprot.2008.75 (2008).
12 Minor, W., Cymborowski, M., Otwinowski, Z. & Chruszcz, M. J. A. C. S. D. B. C. HKL-3000: the integration of data reduction and structure solution—from diffraction images to an initial model in minutes. 62, 859-866 (2006).
13 Vagin, A. & Teplyakov, A. Molecular replacement with MOLREP. Acta Crystallogr D Biol Crystallogr 66, 22-25, doi:10.1107/50907444909042589 (2010).
14 Fu, L. F. Crystal structure of the 2019-nCoV main protease complexed with GC376. To be published, doi:10.2210/pdb7brr/pdb (2020).
15 Owen, C. D. COVID-19 main protease with unliganded active site. TO BE PUBLISHED, doi:10.2210/pdb6yb7/pdb (2020).
16 Ma, C. et al. Boceprevir, GC-376, and calpain inhibitors II, XII inhibit SARS-CoV-2 viral replication by targeting the viral main protease. Cell Research, doi:10.1038/s41422-020-0356-z (2020).
17 Murshudov, G. N. et al. REFMACS for the refinement of macromolecular crystal structures. Acta crystallographica. Section D, Biological crystallography 67, 355-367, doi:10.1107/50907444911001314 (2011).
18 Emsley, P. & Cowtan, K. Coot: model-building tools for molecular graphics. Acta crystallographica. Section D, Biological crystallography 60, 2126-2132, doi:10.1107/s0907444904019158 (2004).

1. A compound encompassed within Formula I: 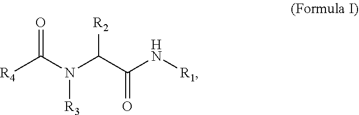
including pharmaceutically acceptable salts, solvates, and/or prodrugs thereof,
wherein each of R1, R2, R3, and R4 independently include any chemical moiety that permits the resulting compound to inhibit M^{pro} protease activity.

2. The compound of claim 1, wherein each of R1, R2, R3, R4, R5, R6, X, Y, Z, *, # if one or both are present, independently include any chemical moiety that permits the resulting compound to treat, ameliorate, and/or prevent viral infection (e.g., COVID-19 infection).

3. The compound of claim 1, wherein R1 is selected from the group consisting of hydrogen, methyl,

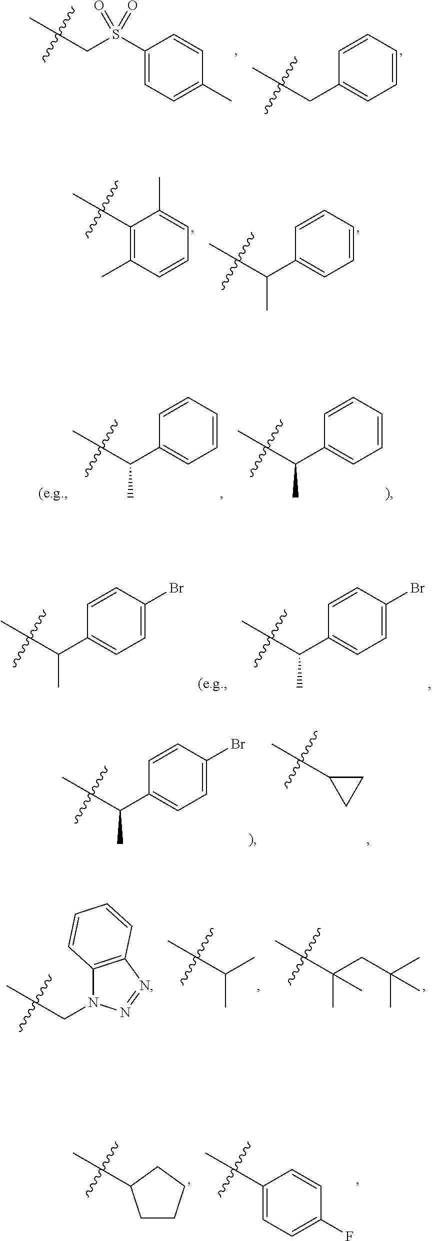

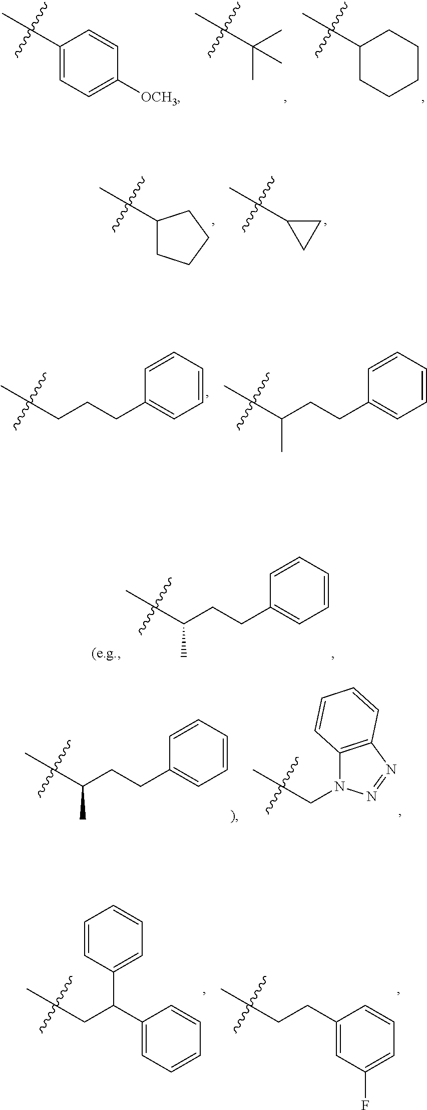

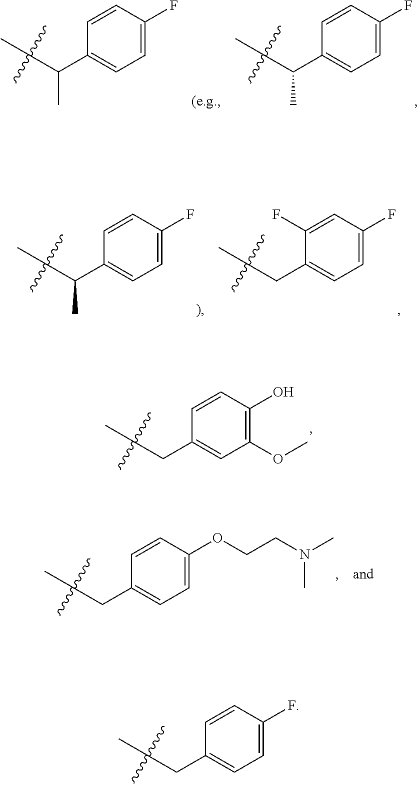

4. The compound of claim 1, wherein R2 is selected from the group consisting of hydrogen,

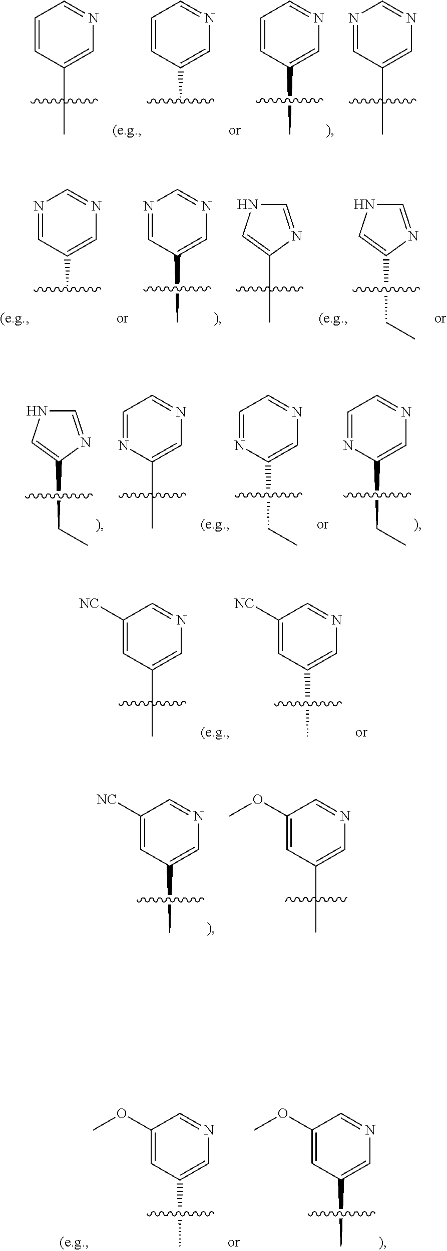

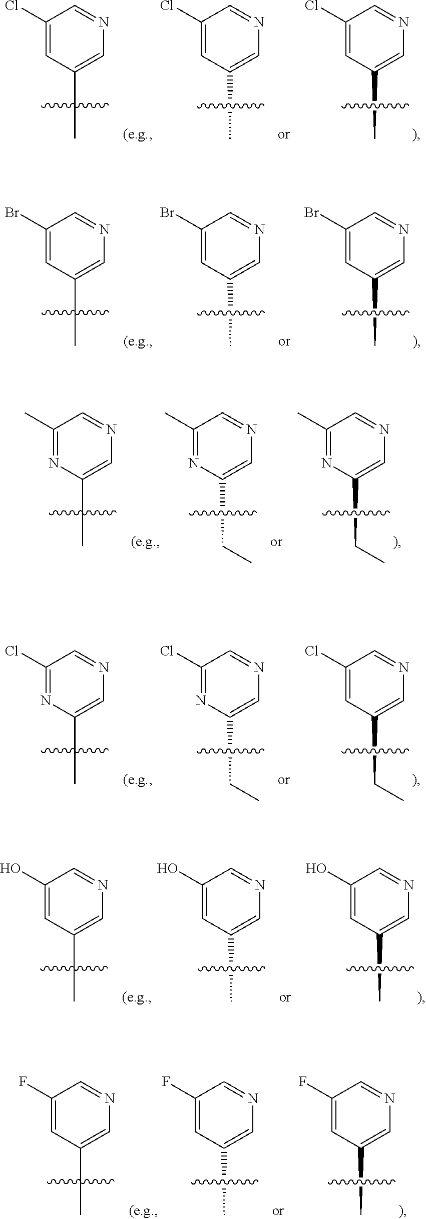

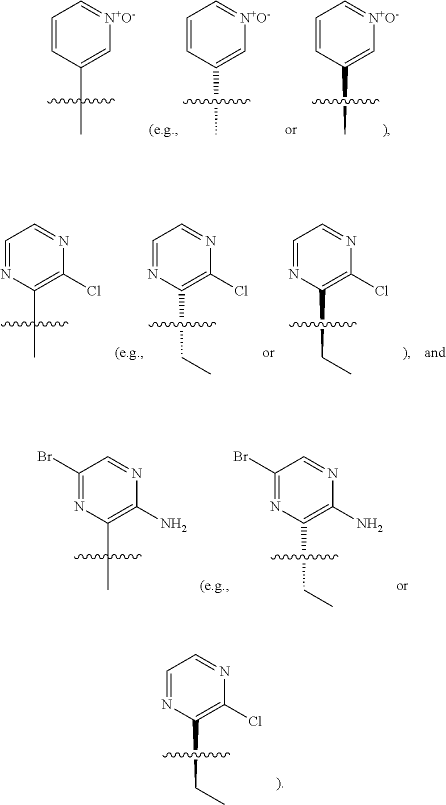

5. The compound of claim 1, wherein R3 is selected from the group consisting of hydrogen,

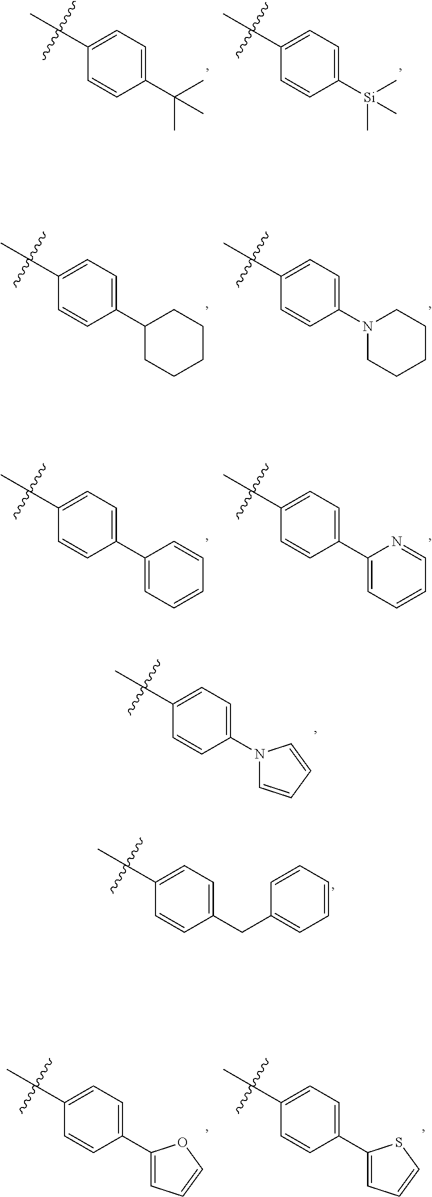

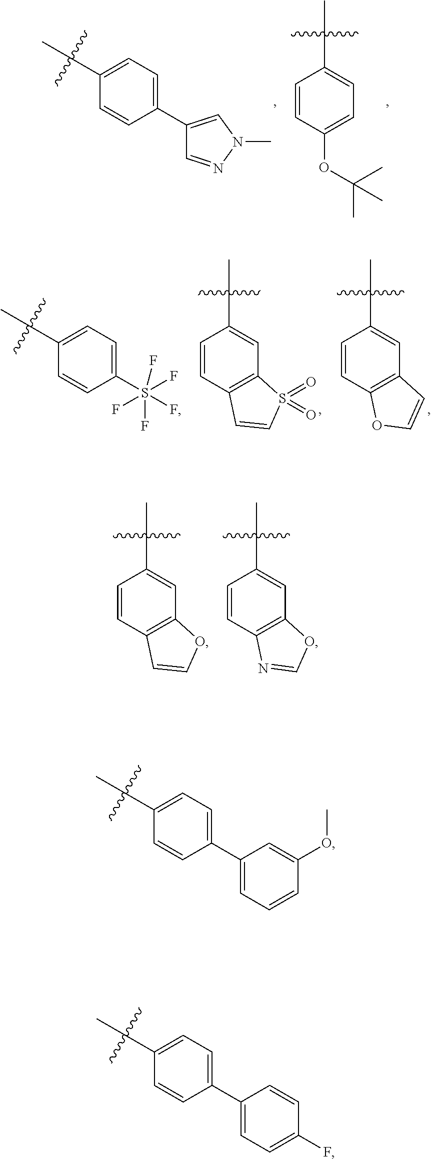

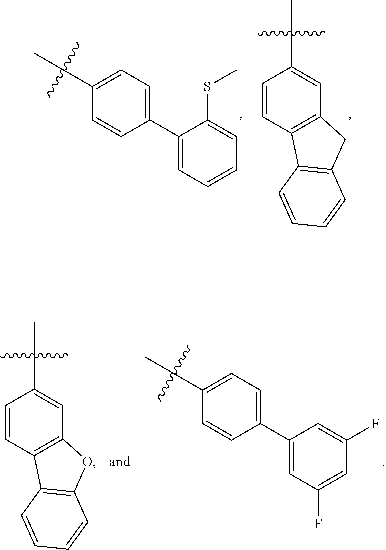

6. The compound of claim 1, wherein R4 is selected from the group consisting of hydrogen,

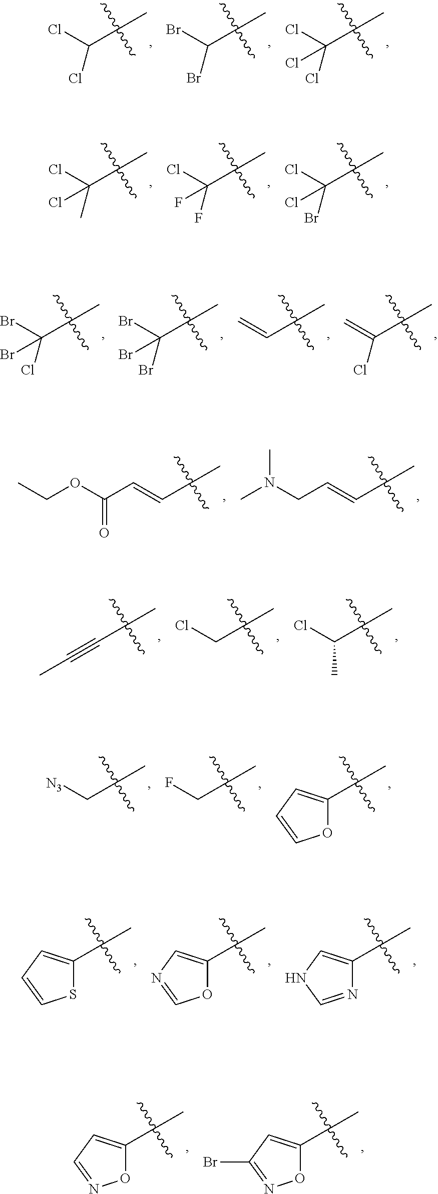

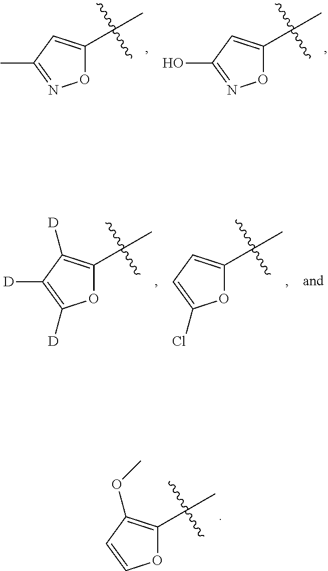

7. The compound of claim 1, wherein said compound is selected from the group of compounds recited in Table 1, FIG. 4, FIG. 10, FIG. 11, and/or FIG. 18.

8. A pharmaceutical composition comprising a compound of claim 1.

9. A method for treating, ameliorating and/or preventing a condition related to viral infection and/or symptoms related to viral infection in a subject, comprising administering to the subject a therapeutically effective amount of the pharmaceutical composition of claim 8.

10. The method of claim 9, wherein the condition related to viral infection is SARS-CoV-2 infection (e.g., COVID-19).

11. The method of claim 9, wherein the subject is a human subject suffering from or at risk of suffering from a condition related to SARS-CoV-2 infection (e.g., COVID-19).

12. The method of claim 9, wherein the pharmaceutical composition is dispersed in a pharmaceutically acceptable carrier.

13. The method of claim 9, wherein the administering results in supression of M^{pro} activity.

14. The method of claim 9, wherein the administering is oral, topical or intravenous.

15. The method of claim 9, further comprising administering to the subject one or more of hydroxychloroquine, dexamethasone, and remdesivir.

16-22. (canceled)

23. The method of claim 9, wherein the symptoms related to viral infection in a subject are one or more of fever, fatigue, dry cough, myalgias, dyspnea, acute respiratory distress syndrome, and pneumonia.

24-61. (canceled)
